(19) United States
(12) Patent Application Publication
(10) Pub. No.: US 2021/0163404 A1
(43) Pub. Date: Jun. 3, 2021

(54) NOVEL UREA COMPOUNDS AND BIOISOSTERES THEREOF AND THEIR USE FOR TREATING INFLAMMATION AND INFLAMMATION-RELATED PATHOLOGIES

(71) Applicant: UNIVERSITÉ LAVAL, Quebec (CA)

(72) Inventors: Rene C. Gaudreault, Levis (CA); Stephane Gobeil, Levis (CA); Jean Rousseau, Quebec (CA)

(21) Appl. No.: 16/618,246

(22) PCT Filed: Jun. 14, 2018
(86) PCT No.: PCT/CA2018/050721

§ 371 Date: Nov. 29, 2019

Related U.S. Application Data
(60) Provisional application No. 62/519,281, filed on Jun. 14, 2017.

(51) Int. Cl. C07C 275/30; C07C 275/28; A61P 37/06; C07C 225/22; C07C 335/16

(52) U.S. Cl. CPC C07C 275/30; C07C 275/28; C07C 335/16; C07C 225/22; A61P 37/06

(57) ABSTRACT

Novel urea, thiourea and squaramide compounds and bioisosteres thereof of formulas (I) and (VI) and the use thereof for treating, attenuating, inhibiting or preventing inflammation and inflammation-related pathologies are described herein.

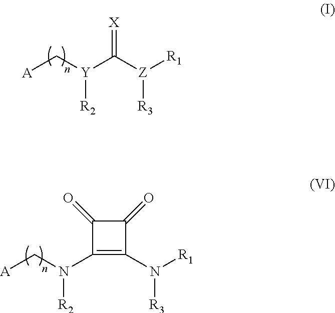

### CROSS REFERENCE TO RELATED APPLICATIONS

This application claims the benefit of U.S. Provisional Application 62/519,281, filed Jun. 14, 2018. The contents of the referenced application are incorporated into the present application by reference.

### FIELD

The present disclosure broadly relates to novel urea compounds and bioisosteres thereof. More specifically, but not exclusively, the present disclosure relates to novel urea compounds, and bioisosteres thereof and pharmaceutical compositions comprising such compounds for treating, attenuating, inhibiting or preventing inflammation and inflammation-related pathologies. Yet more specifically, but not exclusively, the present disclosure relates to novel urea compounds, and bioisosteres thereof and pharmaceutical compositions comprising such compounds for treating, attenuating, inhibiting or preventing conditions associated with the expression of IL-6. The present disclosure also relates to intermediates and processes useful in the synthesis of the urea compounds and bioisosteres thereof.

### BACKGROUND

Phenyl-3-(2-chloroethyl)ureas (CEUs) were developed as soft alkylating agents covalently binding to a number of intracellular proteins. To this end, two prototypical CEUs, namely 3-(2-chloroethyl)-1-p-(tert-butyl)phenyl]urea (tBCEU) [1] and 1-(2-chloroethyl)-3-(p-cyclohexylphenyl)urea (cHCEU) [2, 3] (FIG. 1) were shown to exhibit potent antiproliferative activity on numerous cancer cell lines [4, 5] as well as cancer cell lines having developed mechanisms of chemoresistance [6, 7]. On one hand, the study of the mechanism of action of tBCEU evidenced a unique acylation of Glu198 within the colchicine-binding site on β-tubulin [8] that lead to microtubule depolymerization, cytoskeleton disruption and anoikis of cancer cells [9]. On the other hand, cHCEU acylates prohibitin-1 on Asp40 and thioredoxin-1 on an identified amino acid residue, abrogating the translocation of both proteins from the cytosol to the nucleus [3]. Moreover, tBCEU and cHCEU were shown to exhibit a strong impact on cell cycle progression, arresting cancer cells in the G2/M and G0-G1 phase, respectively. The alkylating properties of CEUs such as tBCEU are due to the presence of a chlorine atom; its absence abrogates the electrophilic properties and the antiproliferative activity of the ethylurea counterparts (e.g. tBEU).

The mechanism of action responsible for the antiproliferative activity of the CEUs has been further investigated and the involvement of the ASK1-P38 signaling pathway in the triggering of cell anoikis was evidenced [10]. Interestingly, a strong link has been established between the P38 signaling pathway and various diseases, notably cancer, inflammation, rheumatoid arthritis and Alzheimer's disease, which depend on the production of cytokines such as IL-6 [11-16]. The inhibition of the synthesis or the release of IL-6 and other pro-inflammatory cytokines (TNFα, IL-1 and IL-2) was previously reported as a potential therapeutic approach for the treatment of diseases associated with inappropriate inflammatory responses [17].

Psoriasis is an inflammatory cutaneous disease that affects 2 to 3% of the world population, both men and women [18]. Several forms of psoriasis have been identified, but the most common form (90% of all cases) is psoriasis vulgaris also called plaques psoriasis [19]. This type of psoriasis is characterised by the presence of whitish and reddish scaly plaques especially on elbows, knees and scalp [20]. The plaques are the result of a hyperproliferation of keratinocytes and, together with their abnormal differentiation, cause a thickening of the epidermis (acanthosis) [21]. The poor epidermal differentiation induces retention of keratinocytes nuclei in the stratum corneum (parakeratosis), in addition to many modifications in protein expression such as involucrin, filaggrin, keratins and loricrin [22-24]. Plaques psoriasis is also characterized by an infiltration of leucocytes in skin and by an increase of angiogenesis producing tortuous, dilated and more permeable capillaries [25-26]. The symptoms of this pathology can be controlled by several treatments; however, no cure is yet available.

### SUMMARY

The present disclosure broadly relates to novel urea, thiourea and squaramide compounds and bioisosteres thereof. In an aspect, the present disclosure relates to novel urea, thioureas and squaramide compounds, and bioisosteres thereof and pharmaceutical compositions comprising such compounds for treating, attenuating, inhibiting or preventing inflammation and inflammation-related pathologies. In a further aspect, the present disclosure relates to novel urea, thioureas and squaramide compounds, and bioisosteres thereof and pharmaceutical compositions comprising such compounds for treating, attenuating, inhibiting or preventing conditions associated with the expression of IL-6. The present disclosure also relates to intermediates and processes useful in the synthesis of the urea, thioureas and squaramide compounds and bioisosteres thereof.

In an aspect, the present disclosure relates to urea compounds and bioisosteres thereof and to their use for treating, attenuating, inhibiting and/or preventing inflammation and inflammation-related pathologies.

In an aspect, the present disclosure relates to urea compounds and bioisosteres thereof and to their use for treating, attenuating, inhibiting and/or preventing conditions associated with the expression of IL-6.

In an aspect, the present disclosure relates to substituted phenyl cycloalkylureas and to their use for treating, attenuating, inhibiting and/or preventing inflammation and inflammation-related pathologies. In an embodiment, the present disclosure relates to substituted phenyl cycloalkylureas and to their use for treating, attenuating, inhibiting and/or preventing conditions associated with the expression of IL-6. In a further embodiment, the present disclosure relates to pharmaceutical compositions comprising one nor more substituted phenyl cycloalkylureas and to their use for treating, attenuating, inhibiting or preventing conditions associated with the expression of IL-6. In a further embodiment, the present disclosure relates to intermediates and processes for the synthesis of substituted phenyl cycloalkylureas.

In an embodiment, the present disclosure relates to a compound of Formula I:

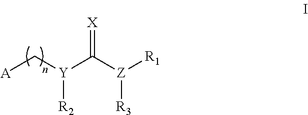

wherein:

A is an arene or a heteroarene;

Y is N, O or S;

Z is N, O or S;

X is O, S or N═CN;

R₁ is a (C₄₋₁₅)-branched alkyl; (C₃₋₈)cycloalkyl; (C₃₋₈)cycloalkyl having at least one substituent selected from (C₁₋₁₀)alkyl, (C₄₋₁₀)branched alkyl, —O—(C₁-C₁₀)alkyl, —S—(C₁-C₁₀)alkyl, —NRR, —NO₂, —CN, —C(O)R, —C(S)R, —C(O)OR, —C(S)OR, —SC(S)R, —OC(S)R, —C(O)NRR, —C(S)NRR, —C(O)NR(OR), —C(S)NR(OR), —C(O)NR(SR), —C(S)NR(SR), —CH(CN)₂, —CH[C(O)R]₂, —CH[C(S)R]₂, —CH[C(O)OR]₂, —CH[C(S)OR]₂, —CH[C(O)SR]₂, —CH[C(S)SR]₂, —NRC(O)R, —NRC(O)OR, —S(O)—R, —S(O)OR, and —S(O)NRR; (C₆₋₁₈)aryl; or (C₆₋₁₈)aryl having at least one substituent selected from (C₁₋₁₀)alkyl, (C₄₋₁₀)branched alkyl, —O—(C₁-C₁₀)alkyl, —S—(C₁-C₁₀)alkyl, —NRR, —NO₂, —CN, —C(O)R, —C(S)R, —C(O)OR, —C(S)OR, —SC(S)R, —OC(S)R, —C(O)NRR, —C(S)NRR, —C(O)NR(OR), —C(S)NR(OR), —C(O)NR(SR), —C(S)NR(SR), —CH(CN)₂, —CH[C(O)R]₂, —CH[C(S)R]₂, —CH[C(O)OR]₂, —CH[C(S)OR]₂, —CH[C(O)SR]₂, —CH[C(S)SR]₂, —NRC(O)R, —NRC(O)OR, —S(O)—R, —S(O)OR, and —S(O)NRR;

R₂ and R₃ are independently hydrogen, (C₁₋₁₀)alkyl, (C₄₋₁₀)branched alkyl, (C₃₋₈)cycloalkyl; (C₃₋₈)cycloalkyl having at least one substituent selected from (C₁₋₁₀)alkyl, (C₄₋₁₀)branched alkyl, —O—(C₁-C₁₀)alkyl, —S—(C₁-C₁₀)alkyl, —NRR, —NO₂, —CN, —C(O)R, —C(S)R, —C(O)OR, —C(S)OR, —SC(S)R, —OC(S)R, —C(O)NRR, —C(S)NRR, —C(O)NR(OR), —C(S)NR(OR), —C(O)NR(SR), —C(S)NR(SR), —CH(CN)₂, —CH[C(O)R]₂, —CH[C(S)R]₂, —CH[C(O)OR]₂, —CH[C(S)OR]₂, —CH[C(O)SR]₂, —CH[C(S)SR]₂, —NRC(O)R, —NRC(O)OR, —S(O)—R, —S(O)OR, and —S(O)NRR; (C₆₋₁₈)aryl; or (C₆₋₁₈)aryl having at least one substituent selected from (C₁₋₁₀)alkyl, (C₄₋₁₀)branched alkyl, —O—(C₁-C₁₀)alkyl, —S—(C₁-C₁₀)alkyl, —NRR, —NO₂, —CN, —C(O)R, —C(S)R, —C(O)OR, —C(S)OR, —SC(S)R, —OC(S)R, —C(O)NRR, —C(S)NRR, —C(O)NR(OR), —C(S)NR(OR), —C(O)NR(SR), —C(S)NR(SR), —CH(CN)₂, —CH[C(O)R]₂, —CH[C(S)R]₂, —CH[C(O)OR]₂, —CH[C(S)OR]₂, —CH[C(O)SR]₂, —CH[C(S)SR]₂, —NRC(O)R, —NRC(O)OR, —S(O)—R, —S(O)OR, and —S(O)NRR; or R₂ and R₃ are linked together to form a nitrogen containing ring system, wherein the nitrogen containing ring system is optionally substituted by at least one substituent selected from (C₁₋₁₀)alkyl, (C₄₋₁₀)branched alkyl, —O—(C₁-C₁₀)alkyl, —S—(C₁-C₁₀)alkyl, —NRR, —NO₂, —CN, —C(O)R, —C(S)R, —C(O)OR, —C(S)OR, —SC(S)R, —OC(S)R, —C(O)NRR, —C(S)NRR, —C(O)NR(OR), —C(S)NR(OR), —C(O)NR(SR), —C(S)NR(SR), —CH(CN)₂, —CH[C(O)R]₂, —CH[C(S)R]₂, —CH[C(O)OR]₂, —CH[C(S)OR]₂, —CH[C(O)SR]₂, —CH[C(S)SR]₂, —NRC(O)R, —NRC(O)OR, —S(O)—R, —S(O)OR, and —S(O)NRR;

n is 0, 1 or 2;
wherein:

A is optionally substituted with one or more substituents selected from the group of (C₁-C₁₅)alkyl, (C₂-C₁₅)alkenyl, (C₂-C₁₅)alkynyl, —O—(C₁-C₁₅)alkyl, —O—(C₂-C₁₅) alkenyl, —O—(C₂-C₁₅) alkynyl, —S—(C₁-C₁₅)alkyl, —S—(C₂-C₁₅)alkenyl, —S—(C₂-C₁₅)alkynyl, (C₃-C₈)cycloalkyl, —O—(C₃-C₈)cycloalkyl, —S—(C₃-C₈)cycloalkyl, -halo, —NRR, —NO₂, —CN, —C(O)R, —C(S)R, —C(O)OR, —C(S)OR, —SC(S)R, —OC(S)R, —C(O)NRR, —C(S)NRR, —C(O)NR(OR), —C(S)NR(OR), —C(O)NR(SR), —C(S)NR(SR), —CH(CN)₂, —CH[C(O)R]₂, —CH[C(S)R]₂, —CH[C(O)OR]₂, —CH[C(S)OR]₂, —CH[C(O)SR]₂, —CH[C(S)SR]₂, —NRC(O)R, —NRC(O)OR, —S(O)—R, —S(O)OR, and —S(O)NRR;
wherein

each R is independently selected from —H, (C₁-C₁₀)alkyl, (C₂-C₁₀)alkenyl, (C₂-C₁₀)alkynyl, (C₃-C₈)cycloalkyl, aryl or substituted aryl;
wherein

when Y and/or Z are O or S, R₂ and/or R₃ are absent;
or a pharmaceutically acceptable salt, a prodrug, a tautomer or a solvate thereof.

In an embodiment, the present disclosure relates to a compound of Formula I:

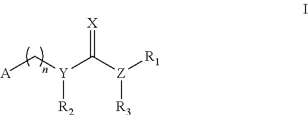

wherein:

A is an arene or a heteroarene;

Y is N, O or S;

Z is N, O or S;

X is O, S or N═CN;

R₁ is a (C₄₋₁₅)-branched alkyl; (C₃₋₈)cycloalkyl; (C₃₋₈)cycloalkyl having at least one substituent selected from (C₁₋₁₀)alkyl, (C₄₋₁₀)branched alkyl, —O—(C₁-C₁₀)alkyl, —S—(C₁-C₁₀)alkyl, —NRR, —NO₂, —CN, —C(O)R, —C(S)R, —C(O)OR, —C(S)OR, —SC(S)R, —OC(S)R, —C(O)NRR, —C(S)NRR, —C(O)NR(OR), —C(S)NR(OR), —C(O)NR(SR), —C(S)NR(SR), —CH(CN)₂, —CH[C(O)R]₂, —CH[C(S)R]₂, —CH[C(O)OR]₂, —CH[C(S)OR]₂, —CH[C(O)SR]₂, —CH[C(S)SR]₂, —NRC(O)R, —NRC(O)OR, —S(O)—R, —S(O)OR, and —S(O)NRR; (C₆₋₁₈)aryl; or (C₆₋₁₈)aryl having at least one substituent selected from (C₁₋₁₀)alkyl, (C₄₋₁₀)branched alkyl, —O—(C₁-C₁₀)alkyl, —S—(C₁-C₁₀)alkyl, —NRR, —NO₂, —CN, —C(O)R, —C(S)R, —C(O)OR, —C(S)OR, —SC(S)R, —OC(S)R, —C(O)NRR, —C(S)NRR, —C(O)NR(OR), —C(S)NR(OR), —C(O)NR(SR), —C(S)NR(SR), —CH(CN)₂, —CH[C(O)R]₂, —CH[C(S)R]₂, —CH[C(O)OR]₂, —CH[C(S)OR]₂, —CH[C(O)SR]₂, —CH[C(S)SR]₂, —NRC(O)R, —NRC(O)OR, —S(O)—R, —S(O)OR, and —S(O)NRR;

R₂ and R₃ are independently hydrogen, (C₁₋₁₀)alkyl, (C₄₋₁₀)branched alkyl, (C₃₋₈)cycloalkyl; (C₃₋₈)cycloalkyl having at least one substituent selected from (C₁₋₁₀)alkyl, (C₄₋₁₀)branched alkyl, —O—(C₁-C₁₀)alkyl, —S—(C₁-C₁₀)alkyl, —NRR, —NO₂, —CN, —C(O)R, —C(S)R, —C(O)OR, —C(S)OR, —SC(S)R, —OC(S)R, —C(O)NRR, —C(S)NRR, —C(O)NR(OR), —C(S)NR(OR), —C(O)NR(SR), —C(S)NR(SR), —CH(CN)₂, —CH[C(O)R]₂, —CH[C(S)R]₂, —CH[C(O)OR]₂, —CH[C(S)OR]₂, —CH[C(O)SR]₂, —CH[C(S)SR]₂, —NRC(O)R, —NRC(O)OR, —S(O)—R, —S(O)OR, and —S(O)NRR; (C₆₋₁₈)aryl; or (C₆₋₁₈)aryl having at least one substituent selected from (C₁₋₁₀)alkyl, (C₄₋₁₀)branched alkyl, —O—(C₁-C₁₀)alkyl, —S—(C₁-C₁₀)alkyl, —NRR, —NO₂, —CN, —C(O)R, —C(S)R, —C(O)OR, —C(S)OR, —SC(S)R, —OC(S)R, —C(O)NRR, —C(S)NRR, —C(O)NR(OR), —C(S)NR(OR), —C(O)NR(SR), —C(S)NR(SR), —CH(CN)₂, —CH[C(O)R]₂, —CH[C(S)R]₂, —CH[C(O)OR]₂, —CH[C(S)OR]₂, —CH[C(O)SR]₂, —CH[C(S)SR]₂, —NRC(O)R, —NRC(O)OR, —S(O)—R, —S(O)OR, and —S(O)NRR; or

R₂ and R₃ are linked together to form a nitrogen containing ring system, wherein the nitrogen containing ring system is optionally substituted by at least one substituent selected from (C₁₋₁₀)alkyl, (C₄₋₁₀)branched alkyl, —O—(C₁-C₁₀)alkyl, —S—(C₁-C₁₀)alkyl, —NRR, —NO₂, —CN, —C(O)R, —C(S)R, —C(O)OR, —C(S)OR, —SC(S)R, —OC(S)R, —C(O)NRR, —C(S)NRR, —C(O)NR(OR), —C(S)NR(OR), —C(O)NR(SR), —C(S)NR(SR), —CH(CN)₂, —CH[C(O)R]₂, —CH[C(S)R]₂, —CH[C(O)OR]₂, —CH[C(S)OR]₂, —CH[C(O)SR]₂, —CH[C(S)SR]₂, —NRC(O)R, —NRC(O)OR, —S(O)—R, —S(O)OR, and —S(O)NRR;

n is 0, 1 or 2;
wherein:

A is optionally substituted with one or more substituents selected from the group of (C₁-C₁₅)alkyl, (C₂-C₁₅)alkenyl, (C₂-C₁₅)alkynyl, —O—(C₁-C₁₅)alkyl, —O—(C₂-C₁₅) alkenyl, —O—(C₂-C₁₅) alkynyl, —S—(C₁-C₁₅)alkyl, —S—(C₂-C₁₅)alkenyl, —S—(C₂-C₁₅)alkynyl, (C₃-C₈)cycloalkyl, —O—(C₃-C₈)cycloalkyl, —S—(C₃-C₈)cycloalkyl, -halo, —NRR, —NO₂, —CN, —C(O)R, —C(S)R, —C(O)OR, —C(S)OR, —SC(S)R, —OC(S)R, —C(O)NRR, —C(S)NRR, —C(O)NR(OR), —C(S)NR(OR), —C(O)NR(SR), —C(S)NR(SR), —CH(CN)₂, —CH[C(O)R]₂, —CH[C(S)R]₂, —CH[C(O)OR]₂, —CH[C(S)OR]₂, —CH[C(O)SR]₂, —CH[C(S)SR]₂, —NRC(O)R, —NRC(O)OR, —S(O)—R, —S(O)OR, and —S(O)NRR;
wherein

each R is independently selected from —H, (C₁-C₁₀)alkyl, (C₂-C₁₀)alkenyl, (C₂-C₁₀)alkynyl, (C₃-C₈)cycloalkyl, aryl or substituted aryl;
wherein

when Y and/or Z are O or S, R₂ and/or R₃ are absent;

or a pharmaceutically acceptable salt, a prodrug, a tautomer or a solvate thereof;
for use as an anti-proliferative agent in the attenuation, inhibition, or prevention of conditions associated with IL-6 expression.

In an embodiment, the present disclosure relates to a compound of Formula I:

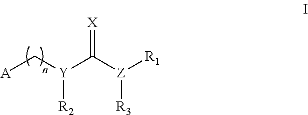

wherein:

A is an arene or a heteroarene;

Y is N, O or S;

Z is N, O or S;

X is O, S or N═CN;

R₁ is a (C₄₋₁₅)-branched alkyl; (C₃₋₈)cycloalkyl; (C₃₋₈)cycloalkyl having at least one substituent selected from (C₁₋₁₀)alkyl, (C₄₋₁₀)branched alkyl, —O—(C₁-C₁₀)alkyl, —S—(C₁-C₁₀)alkyl, —NRR, —NO₂, —CN, —C(O)R, —C(S)R, —C(O)OR, —C(S)OR, —SC(S)R, —OC(S)R, —C(O)NRR, —C(S)NRR, —C(O)NR(OR), —C(S)NR(OR), —C(O)NR(SR), —C(S)NR(SR), —CH(CN)₂, —CH[C(O)R]₂, —CH[C(S)R]₂, —CH[C(O)OR]₂, —CH[C(S)OR]₂, —CH[C(O)SR]₂, —CH[C(S)SR]₂, —NRC(O)R, —NRC(O)OR, —S(O)—R, —S(O)OR, and —S(O)NRR; (C₆₋₁₈)aryl; or (C₆₋₁₈)aryl having at least one substituent selected from (C₁₋₁₀)alkyl, (C₄₋₁₀)branched alkyl, —O—(C₁-C₁₀)alkyl, —S—(C₁-C₁₀)alkyl, —NRR, —NO₂, —CN, —C(O)R, —C(S)R, —C(O)OR, —C(S)OR, —SC(S)R, —OC(S)R, —C(O)NRR, —C(S)NRR, —C(O)NR(OR), —C(S)NR(OR), —C(O)NR(SR), —C(S)NR(SR), —CH(CN)₂, —CH[C(O)R]₂, —CH[C(S)R]₂, —CH[C(O)OR]₂, —CH[C(S)OR]₂, —CH[C(O)SR]₂, —CH[C(S)SR]₂, —NRC(O)R, —NRC(O)OR, —S(O)—R, —S(O)OR, and —S(O)NRR;

R₂ and R₃ are independently hydrogen, (C₁₋₁₀)alkyl, (C₄₋₁₀)branched alkyl, (C₃₋₈)cycloalkyl; (C₃₋₈)cycloalkyl having at least one substituent selected from (C₁₋₁₀)alkyl, (C₄₋₁₀)branched alkyl, —O—(C₁-C₁₀)alkyl, —S—(C₁-C₁₀)alkyl, —NRR, —NO₂, —CN, —C(O)R, —C(S)R, —C(O)OR, —C(S)OR, —SC(S)R, —OC(S)R, —C(O)NRR, —C(S)NRR, —C(O)NR(OR), —C(S)NR(OR), —C(O)NR(SR), —C(S)NR(SR), —CH(CN)₂, —CH[C(O)R]₂, —CH[C(S)R]₂, —CH[C(O)OR]₂, —CH[C(S)OR]₂, —CH[C(O)SR]₂, —CH[C(S)SR]₂, —NRC(O)R, —NRC(O)OR, —S(O)—R, —S(O)OR, and —S(O)NRR; (C₆₋₁₈)aryl; or (C₆₋₁₈)aryl having at least one substituent selected from (C₁₋₁₀)alkyl, (C₄₋₁₀)branched alkyl, —O—(C₁-C₁₀)alkyl, —S—(C₁-C₁₀)alkyl, —NRR, —NO₂, —CN, —C(O)R, —C(S)R, —C(O)OR, —C(S)OR, —SC(S)R, —OC(S)R, —C(O)NRR, —C(S)NRR, —C(O)NR(OR), —C(S)NR(OR), —C(O)NR(SR), —C(S)NR(SR), —CH(CN)₂, —CH[C(O)R]₂, —CH[C(S)R]₂, —CH[C(O)OR]₂, —CH[C(S)OR]₂, —CH[C(O)SR]₂, —CH[C(S)SR]₂, —NRC(O)R, —NRC(O)OR, —S(O)—R, —S(O)OR, and —S(O)NRR; or

R₂ and R₃ are linked together to form a nitrogen containing ring system, wherein the nitrogen containing ring system is optionally substituted by at least one substituent selected from (C₁₋₁₀)alkyl, (C₄₋₁₀)branched alkyl, —O—(C₁-C₁₀)alkyl, —S—(C₁-C₁₀)alkyl, —NRR, —NO₂, —CN, —C(O)R, —C(S)R, —C(O)OR, —C(S)OR, —SC(S)R, —OC(S)R, —C(O)NRR, —C(S)NRR, —C(O)NR(OR), —C(S)NR(OR), —C(O)NR(SR), —C(S)NR(SR), —CH(CN)₂, —CH[C(O)R]₂, —CH[C(S)R]₂, —CH[C(O)OR]₂, —CH[C(S)OR]₂, —CH[C(O)SR]₂, —CH[C(S)SR]₂, —NRC(O)R, —NRC(O)OR, —S(O)—R, —S(O)OR, and —S(O)NRR;

n is 0, 1 or 2;
wherein:

A is optionally substituted with one or more substituents selected from the group of (C₁-C₁₅)alkyl, (C₂-C₁₅)alkenyl, (C₂-C₁₅)alkynyl, —O—(C₁-C₁₅)alkyl, —O—(C₂-C₁₅) alkenyl, —O—(C₂-C₁₅) alkynyl, —S—(C₁-C₁₅)alkyl, —S—(C₂-C₁₅)alkenyl, —S—(C₂-C₁₅)alkynyl, (C₃-C₈)cycloalkyl, —O—(C₃-C₈)cycloalkyl, —S—(C₃-C₈)cycloalkyl, -halo, —NRR, —NO₂, —CN, —C(O)R, —C(S)R, —C(O)OR, —C(S)OR, —SC(S)R, —OC(S)R, —C(O)NRR, —C(S)NRR, —C(O)NR(OR), —C(S)NR(OR), —C(O)NR(SR), —C(S)NR(SR), —CH(CN)₂, —CH[C(O)R]₂, —CH[C(S)R]₂, —CH[C(O)OR]₂, —CH[C(S)OR]₂, —CH[C(O)SR]₂, —CH[C(S)SR]₂, —NRC(O)R, —NRC(O)OR, —S(O)—R, —S(O)OR, and —S(O)NRR;
wherein

each R is independently selected from —H, (C₁-C₁₀)alkyl, (C₂-C₁₀)alkenyl, (C₂-C₁₀)alkynyl, (C₃-C₈)cycloalkyl, aryl or substituted aryl;
wherein

when Y and/or Z are O or S, R₂ and/or R₃ are absent;

or a pharmaceutically acceptable salt, a prodrug, a tautomer or a solvate thereof;
for use as a therapeutic agent in the attenuation, inhibition, or prevention of conditions associated with IL-6 expression.

In an embodiment, the present disclosure relates to a method for treating, attenuating, inhibiting, or preventing a condition associated with IL-6 expression in a subject in need thereof, the method comprising administering a therapeutically effective amount of a compound of Formula I:

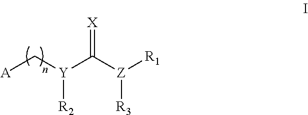

wherein:

A is an arene or a heteroarene;

Y is N, O or S;

Z is N, O or S;

X is O, S or N═CN;

R₁ is a (C₄₋₁₅)-branched alkyl; (C₃₋₈)cycloalkyl; (C₃₋₈)cycloalkyl having at least one substituent selected from (C₁₋₁₀)alkyl, (C₄₋₁₀)branched alkyl, —O—(C₁-C₁₀)alkyl, —S—(C₁-C₁₀)alkyl, —NRR, —NO₂, —CN, —C(O)R, —C(S)R, —C(O)OR, —C(S)OR, —SC(S)R, —OC(S)R, —C(O)NRR, —C(S)NRR, —C(O)NR(OR), —C(S)NR(OR), —C(O)NR(SR), —C(S)NR(SR), —CH(CN)₂, —CH[C(O)R]₂, —CH[C(S)R]₂, —CH[C(O)OR]₂, —CH[C(S)OR]₂, —CH[C(O)SR]₂, —CH[C(S)SR]₂, —NRC(O)R, —NRC(O)OR, —S(O)—R, —S(O)OR, and —S(O)NRR; (C₆₋₁₈)aryl; or (C₆₋₁₈)aryl having at least one substituent selected from (C₁₋₁₀)alkyl, (C₄₋₁₀)branched alkyl, —O—(C₁-C₁₀)alkyl, —S—(C₁-C₁₀)alkyl, —NRR, —NO₂, —CN, —C(O)R, —C(S)R, —C(O)OR, —C(S)OR, —SC(S)R, —OC(S)R, —C(O)NRR, —C(S)NRR, —C(O)NR(OR), —C(S)NR(OR), —C(O)NR(SR), —C(S)NR(SR), —CH(CN)₂, —CH[C(O)R]₂, —CH[C(S)R]₂, —CH[C(O)OR]₂, —CH[C(S)OR]₂, —CH[C(O)SR]₂, —CH[C(S)SR]₂, —NRC(O)R, —NRC(O)OR, —S(O)—R, —S(O)OR, and —S(O)NRR;

R₂ and R₃ are independently hydrogen, (C₁₋₁₀)alkyl, (C₄₋₁₀)branched alkyl, (C₃₋₈)cycloalkyl; (C₃₋₈)cycloalkyl having at least one substituent selected from (C₁₋₁₀)alkyl, (C₄₋₁₀)branched alkyl, —O—(C₁-C₁₀)alkyl, —S—(C₁-C₁₀)alkyl, —NRR, —NO₂, —CN, —C(O)R, —C(S)R, —C(O)OR, —C(S)OR, —SC(S)R, —OC(S)R, —C(O)NRR, —C(S)NRR, —C(O)NR(OR), —C(S)NR(OR), —C(O)NR(SR), —C(S)NR(SR), —CH(CN)₂, —CH[C(O)R]₂, —CH[C(S)R]₂, —CH[C(O)OR]₂, —CH[C(S)OR]₂, —CH[C(O)SR]₂, —CH[C(S)SR]₂, —NRC(O)R, —NRC(O)OR, —S(O)—R, —S(O)OR, and —S(O)NRR; (C₆₋₁₈)aryl; or (C₆₋₁₈)aryl having at least one substituent selected from (C₁₋₁₀)alkyl, (C₄₋₁₀)branched alkyl, —O—(C₁-C₁₀)alkyl, —S—(C₁-C₁₀)alkyl, —NRR, —NO₂, —CN, —C(O)R, —C(S)R, —C(O)OR, —C(S)OR, —SC(S)R, —OC(S)R, —C(O)NRR, —C(S)NRR, —C(O)NR(OR), —C(S)NR(OR), —C(O)NR(SR), —C(S)NR(SR), —CH(CN)₂, —CH[C(O)R]₂, —CH[C(S)R]₂, —CH[C(O)OR]₂, —CH[C(S)OR]₂, —CH[C(O)SR]₂, —CH[C(S)SR]₂, —NRC(O)R, —NRC(O)OR, —S(O)—R, —S(O)OR, and —S(O)NRR; or

R₂ and R₃ are linked together to form a nitrogen containing ring system, wherein the nitrogen containing ring system is optionally substituted by at least one substituent selected from (C₁₋₁₀)alkyl, (C₄₋₁₀)branched alkyl, —O—(C₁-C₁₀)alkyl, —S—(C₁-C₁₀)alkyl, —NRR, —NO₂, —CN, —C(O)R, —C(S)R, —C(O)OR, —C(S)OR, —SC(S)R, —OC(S)R, —C(O)NRR, —C(S)NRR, —C(O)NR(OR), —C(S)NR(OR), —C(O)NR(SR), —C(S)NR(SR), —CH(CN)₂, —CH[C(O)R]₂, —CH[C(S)R]₂, —CH[C(O)OR]₂, —CH[C(S)OR]₂, —CH[C(O)SR]₂, —CH[C(S)SR]₂, —NRC(O)R, —NRC(O)OR, —S(O)—R, —S(O)OR, and —S(O)NRR;

n is 0, 1 or 2;
wherein:

A is optionally substituted with one or more substituents selected from the group of (C₁-C₁₅)alkyl, (C₂-C₁₅)alkenyl, (C₂-C₁₅)alkynyl, —O—(C₁-C₁₅)alkyl, —O—(C₂-C₁₅) alkenyl, —O—(C₂-C₁₅) alkynyl, —S—(C₁-C₁₅)alkyl, —S—(C₂-C₁₅)alkenyl, —S—(C₂-C₁₅)alkynyl, (C₃-C₈)cycloalkyl, —O—(C₃-C₈)cycloalkyl, —S—(C₃-C₈)cycloalkyl, -halo, —NRR, —NO₂, —CN, —C(O)R, —C(S)R, —C(O)OR, —C(S)OR, —SC(S)R, —OC(S)R, —C(O)NRR, —C(S)NRR, —C(O)NR(OR), —C(S)NR(OR), —C(O)NR(SR), —C(S)NR(SR), —CH(CN)₂, —CH[C(O)R]₂, —CH[C(S)R]₂, —CH[C(O)OR]₂, —CH[C(S)OR]₂, —CH[C(O)SR]₂, —CH[C(S)SR]₂, —NRC(O)R, —NRC(O)OR, —S(O)—R, —S(O)OR, and —S(O)NRR;
wherein

each R is independently selected from —H, (C₁-C₁₀)alkyl, (C₂-C₁₀)alkenyl, (C₂-C₁₀)alkynyl, (C₃-C₈)cycloalkyl, aryl or substituted aryl;
wherein

when Y and/or Z are O or S, R₂ and/or R₃ are absent;
or a pharmaceutically acceptable salt, a prodrug, a tautomer or a solvate thereof.

In an embodiment, the present disclosure relates to a pharmaceutical composition comprising a compound of Formula I and a pharmaceutically acceptable carrier.

In an embodiment, the present disclosure relates to a medicament for use in treating, attenuating, inhibiting and/or preventing inflammation and inflammation-related pathologies, the medicament comprising a compound of Formula I and a pharmaceutically acceptable carrier.

In an embodiment, the present disclosure relates to a medicament for use in treating a condition associated with IL-6 expression, the medicament comprising a compound of Formula I and a pharmaceutically acceptable carrier.

In an embodiment, the present disclosure relates to a compound of Formula II:

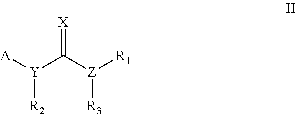

wherein:

A is an arene or a heteroarene;

Y is N, O or S;

Z is N, O or S;

X is O, S or N═CN;

R₁ is a (C₄₋₁₅)-branched alkyl; (C₃₋₈)cycloalkyl; (C₃₋₈)cycloalkyl having at least one substituent selected from (C₁₋₁₀)alkyl, (C₄₋₁₀)branched alkyl, —O—(C₁-C₁₀)alkyl, —S—(C₁-C₁₀)alkyl, —NRR, —NO₂, —CN, —C(O)R, —C(S)R, —C(O)OR, —C(S)OR, —SC(S)R, —OC(S)R, —C(O)NRR, —C(S)NRR, —C(O)NR(OR), —C(S)NR(OR), —C(O)NR(SR), —C(S)NR(SR), —CH(CN)₂, —CH[C(O)R]₂, —CH[C(S)R]₂, —CH[C(O)OR]₂, —CH[C(S)OR]₂, —CH[C(O)SR]₂, —CH[C(S)SR]₂, —NRC(O)R, —NRC(O)OR, —S(O)—R, —S(O)OR, and —S(O)NRR; (C₆₋₁₈)aryl; or (C₆₋₁₈)aryl having at least one substituent selected from (C₁₋₁₀)alkyl, (C₄₋₁₀)branched alkyl, —O—(C₁-C₁₀)alkyl, —S—(C₁-C₁₀)alkyl, —NRR, —NO₂, —CN, —C(O)R, —C(S)R, —C(O)OR, —C(S)OR, —SC(S)R, —OC(S)R, —C(O)NRR, —C(S)NRR, —C(O)NR(OR), —C(S)NR(OR), —C(O)NR(SR), —C(S)NR(SR), —CH(CN)₂, —CH[C(O)R]₂, —CH[C(S)R]₂, —CH[C(O)OR]₂, —CH[C(S)OR]₂, —CH[C(O)SR]₂, —CH[C(S)SR]₂, —NRC(O)R, —NRC(O)OR, —S(O)—R, —S(O)OR, and —S(O)NRR;

R₂ and R₃ are independently hydrogen, (C₁₋₁₀)alkyl, (C₄₋₁₀)branched alkyl, (C₃₋₈)cycloalkyl; (C₃₋₈)cycloalkyl having at least one substituent selected from (C₁₋₁₀)alkyl, (C₄₋₁₀)branched alkyl, —O—(C₁-C₁₀)alkyl, —S—(C₁-C₁₀)alkyl, —NRR, —NO₂, —CN, —C(O)R, —C(S)R, —C(O)OR, —C(S)OR, —SC(S)R, —OC(S)R, —C(O)NRR, —C(S)NRR, —C(O)NR(OR), —C(S)NR(OR), —C(O)NR(SR), —C(S)NR(SR), —CH(CN)₂, —CH[C(O)R]₂, —CH[C(S)R]₂, —CH[C(O)OR]₂, —CH[C(S)OR]₂, —CH[C(O)SR]₂, —CH[C(S)SR]₂, —NRC(O)R, —NRC(O)OR, —S(O)—R, —S(O)OR, and —S(O)NRR; (C₁₋₁₈)aryl; or (C₆₋₁₈)aryl having at least one substituent selected from (C₁₋₁₀)alkyl, (C₄₋₁₀)branched alkyl, —O—(C₁-C₁₀)alkyl, —S—(C₁-C₁₀)alkyl, —NRR, —NO₂, —CN, —C(O)R, —C(S)R, —C(O)OR, —C(S)OR, —SC(S)R, —OC(S)R, —C(O)NRR, —C(S)NRR, —C(O)NR(OR), —C(S)NR(OR), —C(O)NR(SR), —C(S)NR(SR), —CH(CN)₂, —CH[C(O)R]₂, —CH[C(S)R]₂, —CH[C(O)OR]₂, —CH[C(S)OR]₂, —CH[C(O)SR]₂, —CH[C(S)SR]₂, —NRC(O)R, —NRC(O)OR, —S(O)—R, —S(O)OR, and —S(O)NRR; or

R₂ and R₃ are linked together to form a nitrogen containing ring system, wherein the nitrogen containing ring system is optionally substituted by at least one substituent selected from (C₁₋₁₀)alkyl, (C₄₋₁₀)branched alkyl, —O—(C₁-C₁₀)alkyl, —S—(C₁-C₁₀)alkyl, —NRR, —NO₂, —CN, —C(O)R, —C(S)R, —C(O)OR, —C(S)OR, —SC(S)R, —OC(S)R, —C(O)NRR, —C(S)NRR, —C(O)NR(OR), —C(S)NR(OR), —C(O)NR(SR), —C(S)NR(SR), —CH(CN)₂, —CH[C(O)R]₂, —CH[C(S)R]₂, —CH[C(O)OR]₂, —CH[C(S)OR]₂, —CH[C(O)SR]₂, —CH[C(S)SR]₂, —NRC(O)R, —NRC(O)OR, —S(O)—R, —S(O)OR, and —S(O)NRR;
wherein:

A is optionally substituted with one or more substituents selected from the group of (C₁-C₁₅)alkyl, (C₂-C₁₅)alkenyl, (C₂-C₁₅)alkynyl, —O—(C₁-C₁₅)alkyl, —O—(C₂-C₁₅) alkenyl, —O—(C₂-C₁₅) alkynyl, —S—(C₁-C₁₅)alkyl, —S—(C₂-C₁₅)alkenyl, —S—(C₂-C₁₅)alkynyl, (C₃-C₈)cycloalkyl, —O—(C₃-C₈)cycloalkyl, —S—(C₃-C₈)cycloalkyl, -halo, —NRR, —NO₂, —CN, —C(O)R, —C(S)R, —C(O)OR, —C(S)OR, —SC(S)R, —OC(S)R, —C(O)NRR, —C(S)NRR, —C(O)NR(OR), —C(S)NR(OR), —C(O)NR(SR), —C(S)NR(SR), —CH(CN)₂, —CH[C(O)R]₂, —CH[C(S)R]₂, —CH[C(O)OR]₂, —CH[C(S)OR]₂, —CH[C(O)SR]₂, —CH[C(S)SR]₂, —NRC(O)R, —NRC(O)OR, —S(O)—R, —S(O)OR, and —S(O)NRR;
wherein

each R is independently selected from —H, (C₁-C₁₀)alkyl, (C₂-C₁₀)alkenyl, (C₂-C₁₀)alkynyl, (C₃-C₈)cycloalkyl, aryl or substituted aryl;
wherein

when Y and/or Z are 0 or S, R₂ and/or R₃ are absent;
or a pharmaceutically acceptable salt, a prodrug, a tautomer or a solvate thereof.

In an embodiment, the present disclosure relates to a compound of Formula II:

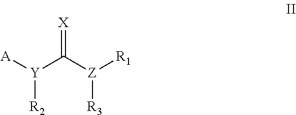

wherein:

A is an arene or a heteroarene;

Y is N, O or S;

Z is N, O or S;

X is O, S or N═CN;

R₁ is a (C₄₋₁₅)-branched alkyl; (C₃₋₈)cycloalkyl; (C₃₋₈)cycloalkyl having at least one substituent selected from (C₁₋₁₀)alkyl, (C₄₋₁₀)branched alkyl, —O—(C₁-C₁₀)alkyl, —S—(C₁-C₁₀)alkyl, —NRR, —NO₂, —CN, —C(O)R, —C(S)R, —C(O)OR, —C(S)OR, —SC(S)R, —OC(S)R, —C(O)NRR, —C(S)NRR, —C(O)NR(OR), —C(S)NR(OR), —C(O)NR(SR), —C(S)NR(SR), —CH(CN)₂, —CH[C(O)R]₂, —CH[C(S)R]₂, —CH[C(O)OR]₂, —CH[C(S)OR]₂, —CH[C(O)SR]₂, —CH[C(S)SR]₂, —NRC(O)R, —NRC(O)OR, —S(O)—R, —S(O)OR, and —S(O)NRR; (C₆₋₁₈)aryl; or (C₆₋₁₈)aryl having at least one substituent selected from (C₁₋₁₀)alkyl, (C₄₋₁₀)branched alkyl, —O—(C₁-C₁₀)alkyl, —S—(C₁-C₁₀)alkyl, —NRR, —NO₂, —CN, —C(O)R, —C(S)R, —C(O)OR, —C(S)OR, —SC(S)R, —OC(S)R, —C(O)NRR, —C(S)NRR, —C(O)NR(OR), —C(S)NR(OR), —C(O)NR(SR), —C(S)NR(SR), —CH(CN)₂, —CH[C(O)R]₂, —CH[C(S)R]₂, —CH[C(O)OR]₂, —CH[C(S)OR]₂, —CH[C(O)SR]₂, —CH[C(S)SR]₂, —NRC(O)R, —NRC(O)OR, —S(O)—R, —S(O)OR, and —S(O)NRR;

R₂ and R₃ are independently hydrogen, (C₁₋₁₀)alkyl, (C₄₋₁₀)branched alkyl, (C₃₋₈)cycloalkyl; (C₃₋₈)cycloalkyl having at least one substituent selected from (C₁₋₁₀)alkyl, (C₄₋₁₀)branched alkyl, —O—(C₁-C₁₀)alkyl, —S—(C₁-C₁₀)alkyl, —NRR, —NO₂, —CN, —C(O)R, —C(S)R, —C(O)OR, —C(S)OR, —SC(S)R, —OC(S)R, —C(O)NRR, —C(S)NRR, —C(O)NR(OR), —C(S)NR(OR), —C(O)NR(SR), —C(S)NR(SR), —CH(CN)₂, —CH[C(O)R]₂, —CH[C(S)R]₂, —CH[C(O)OR]₂, —CH[C(S)OR]₂, —CH[C(O)SR]₂, —CH[C(S)SR]₂, —NRC(O)R, —NRC(O)OR, —S(O)—R, —S(O)OR, and —S(O)NRR; (C₆₋₁₈)aryl; or (C₆₋₁₈)aryl having at least one substituent selected from (C₁₋₁₀)alkyl, (C₄₋₁₀)branched alkyl, —O—(C₁-C₁₀)alkyl, —S—(C₁-C₁₀)alkyl, —NRR, —NO₂, —CN, —C(O)R, —C(S)R, —C(O)OR, —C(S)OR, —SC(S)R, —OC(S)R, —C(O)NRR, —C(S)NRR, —C(O)NR(OR), —C(S)NR(OR), —C(O)NR(SR), —C(S)NR(SR), —CH(CN)₂, —CH[C(O)R]₂, —CH[C(S)R]₂, —CH[C(O)OR]₂, —CH[C(S)OR]₂, —CH[C(O)SR]₂, —CH[C(S)SR]₂, —NRC(O)R, —NRC(O)OR, —S(O)—R, —S(O)OR, and —S(O)NRR; or

R₂ and R₃ are linked together to form a nitrogen containing ring system, wherein the nitrogen containing ring system is optionally substituted by at least one substituent selected from (C₁₋₁₀)alkyl, (C₄₋₁₀)branched alkyl, —O—(C₁-C₁₀)alkyl, —S—(C₁-C₁₀)alkyl, —NRR, —NO₂, —CN, —C(O)R, —C(S)R, —C(O)OR, —C(S)OR, —SC(S)R, —OC(S)R, —C(O)NRR, —C(S)NRR, —C(O)NR(OR), —C(S)NR(OR), —C(O)NR(SR), —C(S)NR(SR), —CH(CN)₂, —CH[C(O)R]₂, —CH[C(S)R]₂, —CH[C(O)OR]₂, —CH[C(S)OR]₂, —CH[C(O)SR]₂, —CH[C(S)SR]₂, —NRC(O)R, —NRC(O)OR, —S(O)—R, —S(O)OR, and —S(O)NRR;
wherein:

A is optionally substituted with one or more substituents selected from the group of (C₁-C₁₅)alkyl, (C₂-C₁₅)alkenyl, (C₂-C₁₅)alkynyl, —O—(C₁-C₁₅)alkyl, —O—(C₂-C₁₅) alkenyl, —O—(C₂-C₁₅) alkynyl, —S—(C₁-C₁₅)alkyl, —S—(C₂-C₁₅)alkenyl, —S—(C₂-C₁₅)alkynyl, (C₃-C₈)cycloalkyl, —O—(C₃-C₈)cycloalkyl, —S—(C₃-C₈)cycloalkyl, -halo, —NRR, —NO₂, —CN, —C(O)R, —C(S)R, —C(O)OR, —C(S)OR, —SC(S)R, —OC(S)R, —C(O)NRR, —C(S)NRR, —C(O)NR(OR), —C(S)NR(OR), —C(O)NR(SR), —C(S)NR(SR), —CH(CN)₂, —CH[C(O)R]₂, —CH[C(S)R]₂, —CH[C(O)OR]₂, —CH[C(S)OR]₂, —CH[C(O)SR]₂, —CH[C(S)SR]₂, —NRC(O)R, —NRC(O)OR, —S(O)—R, —S(O)OR, and —S(O)NRR;
wherein

each R is independently selected from —H, (C₁-C₁₀)alkyl, (C₂-C₁₀)alkenyl, (C₂-C₁₀)alkynyl, (C₃-C₈)cycloalkyl, aryl or substituted aryl;
wherein

when Y and/or Z are O or S, R₂ and/or R₃ are absent;

or a pharmaceutically acceptable salt, a prodrug, a tautomer or a solvate thereof; for use as an anti-proliferative agent in the attenuation, inhibition, or prevention of conditions associated with IL-6 expression.

In an embodiment, the present disclosure relates to a compound of Formula II:

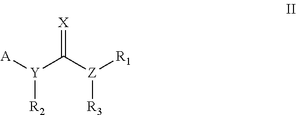

wherein:

A is an arene or a heteroarene;

Y is N, O or S;

Z is N, O or S;

X is O, S or N═CN;

R₁ is a (C₄₋₁₅)-branched alkyl; (C₃₋₈)cycloalkyl; (C₃₋₈)cycloalkyl having at least one substituent selected from (C₁₋₁₀)alkyl, (C₄₋₁₀)branched alkyl, —O—(C₁-C₁₀)alkyl, —S—(C₁-C₁₀)alkyl, —NRR, —NO₂, —CN, —C(O)R, —C(S)R, —C(O)OR, —C(S)OR, —SC(S)R, —OC(S)R, —C(O)NRR, —C(S)NRR, —C(O)NR(OR), —C(S)NR(OR), —C(O)NR(SR), —C(S)NR(SR), —CH(CN)₂, —CH[C(O)R]₂, —CH[C(S)R]₂, —CH[C(O)OR]₂, —CH[C(S)OR]₂, —CH[C(O)SR]₂, —CH[C(S)SR]₂, —NRC(O)R, —NRC(O)OR, —S(O)—R, —S(O)OR, and —S(O)NRR; (C₆₋₁₈)aryl; or (C₆₋₁₈)aryl having at least one substituent selected from (C₁₋₁₀)alkyl, (C₄₋₁₀)branched alkyl, —O—(C₁-C₁₀)alkyl, —S—(C₁-C₁₀)alkyl, —NRR, —NO₂, —CN, —C(O)R, —C(S)R, —C(O)OR, —C(S)OR, —SC(S)R, —OC(S)R, —C(O)NRR, —C(S)NRR, —C(O)NR(OR), —C(S)NR(OR), —C(O)NR(SR), —C(S)NR(SR), —CH(CN)₂, —CH[C(O)R]₂, —CH[C(S)R]₂, —CH[C(O)OR]₂, —CH[C(S)OR]₂, —CH[C(O)SR]₂, —CH[C(S)SR]₂, —NRC(O)R, —NRC(O)OR, —S(O)—R, —S(O)OR, and —S(O)NRR;

R₂ and R₃ are independently hydrogen, (C₁₋₁₀)alkyl, (C₄₋₁₀)branched alkyl, (C₃₋₈)cycloalkyl; (C₃₋₈)cycloalkyl having at least one substituent selected from (C₁₋₁₀)alkyl, (C₄₋₁₀)branched alkyl, —O—(C₁-C₁₀)alkyl, —S—(C₁-C₁₀)alkyl, —NRR, —NO₂, —CN, —C(O)R, —C(S)R, —C(O)OR, —C(S)OR, —SC(S)R, —OC(S)R, —C(O)NRR, —C(S)NRR, —C(O)NR(OR), —C(S)NR(OR), —C(O)NR(SR), —C(S)NR(SR), —CH(CN)₂, —CH[C(O)R]₂, —CH[C(S)R]₂, —CH[C(O)OR]₂, —CH[C(S)OR]₂, —CH[C(O)SR]₂, —CH[C(S)SR]₂, —NRC(O)R, —NRC(O)OR, —S(O)—R, —S(O)OR, and —S(O)NRR; (C₁₋₁₈)aryl; or (C₆₋₁₈)aryl having at least one substituent selected from (C₁₋₁₀)alkyl, (C₄₋₁₀)branched alkyl, —O—(C₁-C₁₀)alkyl, —S—(C₁-C₁₀)alkyl, —NRR, —NO₂, —CN, —C(O)R, —C(S)R, —C(O)OR, —C(S)OR, —SC(S)R, —OC(S)R, —C(O)NRR, —C(S)NRR, —C(O)NR(OR), —C(S)NR(OR), —C(O)NR(SR), —C(S)NR(SR), —CH(CN)₂, —CH[C(O)R]₂, —CH[C(S)R]₂, —CH[C(O)OR]₂, —CH[C(S)OR]₂, —CH[C(O)SR]₂, —CH[C(S)SR]₂, —NRC(O)R, —NRC(O)OR, —S(O)—R, —S(O)OR, and —S(O)NRR; or

R₂ and R₃ are linked together to form a nitrogen containing ring system, wherein the nitrogen containing ring system is optionally substituted by at least one substituent selected from (C₁₋₁₀)alkyl, (C₄₋₁₀)branched alkyl, —O—(C₁-C₁₀)alkyl, —S—(C₁-C₁₀)alkyl, —NRR, —NO₂, —CN, —C(O)R, —C(S)R, —C(O)OR, —C(S)OR, —SC(S)R, —OC(S)R, —C(O)NRR, —C(S)NRR, —C(O)NR(OR), —C(S)NR(OR), —C(O)NR(SR), —C(S)NR(SR), —CH(CN)₂, —CH[C(O)R]₂, —CH[C(S)R]₂, —CH[C(O)OR]₂, —CH[C(S)OR]₂, —CH[C(O)SR]₂, —CH[C(S)SR]₂, —NRC(O)R, —NRC(O)OR, —S(O)—R, —S(O)OR, and —S(O)NRR;
wherein:

A is optionally substituted with one or more substituents selected from the group of (C₁-C₁₅)alkyl, (C₂-C₁₅)alkenyl, (C₂-C₁₅)alkynyl, —O—(C₁-C₁₅)alkyl, —O—(C₂-C₁₅) alkenyl, —O—(C₂-C₁₅) alkynyl, —S—(C₁-C₁₅)alkyl, —S—(C₂-C₁₅)alkenyl, —S—(C₂-C₁₅)alkynyl, (C₃-C₈)cycloalkyl, —O—(C₃-C₈)cycloalkyl, —S—(C₃-C₈)cycloalkyl, -halo, —NRR, —NO₂, —CN, —C(O)R, —C(S)R, —C(O)OR, —C(S)OR, —SC(S)R, —OC(S)R, —C(O)NRR, —C(S)NRR, —C(O)NR(OR), —C(S)NR(OR), —C(O)NR(SR), —C(S)NR(SR), —CH(CN)₂, —CH[C(O)R]₂, —CH[C(S)R]₂, —CH[C(O)OR]₂, —CH[C(S)OR]₂, —CH[C(O)SR]₂, —CH[C(S)SR]₂, —NRC(O)R, —NRC(O)OR, —S(O)—R, —S(O)OR, and —S(O)NRR;
wherein

each R is independently selected from —H, (C₁-C₁₀)alkyl, (C₂-C₁₀)alkenyl, (C₂-C₁₀)alkynyl, (C₃-C₈)cycloalkyl, aryl or substituted aryl;
wherein

when Y and/or Z are O or S, R₂ and/or R₃ are absent;

or a pharmaceutically acceptable salt, a prodrug, a tautomer or a solvate thereof;

for use as a therapeutic agent in the attenuation, inhibition, or prevention of conditions associated with IL-6 expression.

In an embodiment, the present disclosure relates to a method for treating, attenuating, inhibiting, or preventing a condition associated with IL-6 expression in a subject in need thereof, the method comprising administering a therapeutically effective amount of a compound of Formula II:

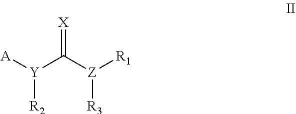

wherein:

A is an arene or a heteroarene;

Y is N, O or S;

Z is N, O or S;

X is O, S or N═CN;

R₁ is a (C₄₋₁₅)-branched alkyl; (C₃₋₈)cycloalkyl; (C₃₋₈)cycloalkyl having at least one substituent selected from (C₁₋₁₀)alkyl, (C₄₋₁₀)branched alkyl, —O—(C₁-C₁₀)alkyl, —S—(C₁-C₁₀)alkyl, —NRR, —NO₂, —CN, —C(O)R, —C(S)R, —C(O)OR, —C(S)OR, —SC(S)R, —OC(S)R, —C(O)NRR, —C(S)NRR, —C(O)NR(OR), —C(S)NR(OR), —C(O)NR(SR), —C(S)NR(SR), —CH(CN)₂, —CH[C(O)R]₂, —CH[C(S)R]₂, —CH[C(O)OR]₂, —CH[C(S)OR]₂, —CH[C(O)SR]₂, —CH[C(S)SR]₂, —NRC(O)R, —NRC(O)OR, —S(O)—R, —S(O)OR, and —S(O)NRR; (C₆₋₁₈)aryl; or (C₆₋₁₈)aryl having at least one substituent selected from (C₁₋₁₀)alkyl, (C₄₋₁₀)branched alkyl, —O—(C₁-C₁₀)alkyl, —S—(C₁-C₁₀)alkyl, —NRR, —NO₂, —CN, —C(O)R, —C(S)R, —C(O)OR, —C(S)OR, —SC(S)R, —OC(S)R, —C(O)NRR, —C(S)NRR, —C(O)NR(OR), —C(S)NR(OR), —C(O)NR(SR), —C(S)NR(SR), —CH(CN)₂, —CH[C(O)R]₂, —CH[C(S)R]₂, —CH[C(O)OR]₂, —CH[C(S)OR]₂, —CH[C(O)SR]₂, —CH[C(S)SR]₂, —NRC(O)R, —NRC(O)OR, —S(O)—R, —S(O)OR, and —S(O)NRR;

R₂ and R₃ are independently hydrogen, (C₁₋₁₀)alkyl, (C₄₋₁₀)branched alkyl, (C₃₋₈)cycloalkyl; (C₃₋₈)cycloalkyl having at least one substituent selected from (C₁₋₁₀)alkyl, (C₄₋₁₀)branched alkyl, —O—(C₁-C₁₀)alkyl, —S—(C₁-C₁₀)alkyl, —NRR, —NO₂, —CN, —C(O)R, —C(S)R, —C(O)OR, —C(S)OR, —SC(S)R, —OC(S)R, —C(O)NRR, —C(S)NRR, —C(O)NR(OR), —C(S)NR(OR), —C(O)NR(SR), —C(S)NR(SR), —CH(CN)₂, —CH[C(O)R]₂, —CH[C(S)R]₂, —CH[C(O)OR]₂, —CH[C(S)OR]₂, —CH[C(O)SR]₂, —CH[C(S)SR]₂, —NRC(O)R, —NRC(O)OR, —S(O)—R, —S(O)OR, and —S(O)NRR; (C₁₋₁₈)aryl; or (C₆₋₁₈)aryl having at least one substituent selected from (C₁₋₁₀)alkyl, (C₄₋₁₀)branched alkyl, —O—(C₁-C₁₀)alkyl, —S—(C₁-C₁₀)alkyl, —NRR, —NO₂, —CN, —C(O)R, —C(S)R, —C(O)OR, —C(S)OR, —SC(S)R, —OC(S)R, —C(O)NRR, —C(S)NRR, —C(O)NR(OR), —C(S)NR(OR), —C(O)NR(SR), —C(S)NR(SR), —CH(CN)₂, —CH[C(O)R]₂, —CH[C(S)R]₂, —CH[C(O)OR]₂, —CH[C(S)OR]₂, —CH[C(O)SR]₂, —CH[C(S)SR]₂, —NRC(O)R, —NRC(O)OR, —S(O)—R, —S(O)OR, and —S(O)NRR; or

R₂ and R₃ are linked together to form a nitrogen containing ring system, wherein the nitrogen containing ring system is optionally substituted by at least one substituent selected from (C₁₋₁₀)alkyl, (C₄₋₁₀)branched alkyl, —O—(C₁-C₁₀)alkyl, —S—(C₁-C₁₀)alkyl, —NRR, —NO₂, —CN, —C(O)R, —C(S)R, —C(O)OR, —C(S)OR, —SC(S)R, —OC(S)R, —C(O)NRR, —C(S)NRR, —C(O)NR(OR), —C(S)NR(OR), —C(O)NR(SR), —C(S)NR(SR), —CH(CN)₂, —CH[C(O)R]₂, —CH[C(S)R]₂, —CH[C(O)OR]₂, —CH[C(S)OR]₂, —CH[C(O)SR]₂, —CH[C(S)SR]₂, —NRC(O)R, —NRC(O)OR, —S(O)—R, —S(O)OR, and —S(O)NRR;
wherein:

A is optionally substituted with one or more substituents selected from the group of (C₁-C₁₅)alkyl, (C₂-C₁₅)alkenyl, (C₂-C₁₅)alkynyl, —O—(C₁-C₁₅)alkyl, —O—(C₂-C₁₅) alkenyl, —O—(C₂-C₁₅) alkynyl, —S—(C₁-C₁₅)alkyl, —S—(C₂-C₁₅)alkenyl, —S—(C₂-C₁₅)alkynyl, (C₃-C₈)cycloalkyl, —O—(C₃-C₈)cycloalkyl, —S—(C₃-C₈)cycloalkyl, -halo, —NRR, —NO₂, —CN, —C(O)R, —C(S)R, —C(O)OR, —C(S)OR, —SC(S)R, —OC(S)R, —C(O)NRR, —C(S)NRR, —C(O)NR(OR), —C(S)NR(OR), —C(O)NR(SR), —C(S)NR(SR), —CH(CN)₂, —CH[C(O)R]₂, —CH[C(S)R]₂, —CH[C(O)OR]₂, —CH[C(S)OR]₂, —CH[C(O)SR]₂, —CH[C(S)SR]₂, —NRC(O)R, —NRC(O)OR, —S(O)—R, —S(O)OR, and —S(O)NRR;
wherein

each R is independently selected from —H, (C₁-C₁₀)alkyl, (C₂-C₁₀)alkenyl, (C₂-C₁₀)alkynyl, (C₃-C₈)cycloalkyl, aryl or substituted aryl;
wherein

when Y and/or Z are O or S, R₂ and/or R₃ are absent;
or a pharmaceutically acceptable salt, a prodrug, a tautomer or a solvate thereof.

In an embodiment, the present disclosure relates to a pharmaceutical composition comprising a compound of Formula II and a pharmaceutically acceptable carrier.

In an embodiment, the present disclosure relates to a medicament for use in treating, attenuating, inhibiting and/or preventing inflammation and inflammation-related pathologies, the medicament comprising a compound of Formula II and a pharmaceutically acceptable carrier.

In an embodiment, the present disclosure relates to a medicament for use in treating a condition associated with IL-6 expression, the medicament comprising a compound of Formula II and a pharmaceutically acceptable carrier.

In an embodiment, the present disclosure relates to a compound of Formula III:

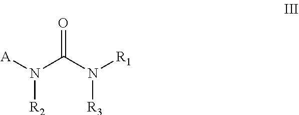

wherein:

A is an arene or a heteroarene;

R₁ is a (C₄₋₁₅)-branched alkyl; (C₃₋₈)cycloalkyl; (C₃₋₈)cycloalkyl having at least one substituent selected from (C₁₋₁₀)alkyl, (C₄₋₁₀)branched alkyl, —O—(C₁-C₁₀)alkyl, —S—(C₁-C₁₀)alkyl, —NRR, —NO₂, —CN, —C(O)R, —C(S)R, —C(O)OR, —C(S)OR, —SC(S)R, —OC(S)R, —C(O)NRR, —C(S)NRR, —C(O)NR(OR), —C(S)NR(OR), —C(O)NR(SR), —C(S)NR(SR), —CH(CN)₂, —CH[C(O)R]₂, —CH[C(S)R]₂, —CH[C(O)OR]₂, —CH[C(S)OR]₂, —CH[C(O)SR]₂, —CH[C(S)SR]₂, —NRC(O)R, —NRC(O)OR, —S(O)—R, —S(O)OR, and —S(O)NRR; (C₆₋₁₈)aryl; or (C₆₋₁₈)aryl having at least one substituent selected from (C₁₋₁₀)alkyl, (C₄₋₁₀)branched alkyl, —O—(C₁-C₁₀)alkyl, —S—(C₁-C₁₀)alkyl, —NRR, —NO₂, —CN, —C(O)R, —C(S)R, —C(O)OR, —C(S)OR, —SC(S)R, —OC(S)R, —C(O)NRR, —C(S)NRR, —C(O)NR(OR), —C(S)NR(OR), —C(O)NR(SR), —C(S)NR(SR), —CH(CN)₂, —CH[C(O)R]₂, —CH[C(S)R]₂, —CH[C(O)OR]₂, —CH[C(S)OR]₂, —CH[C(O)SR]₂, —CH[C(S)SR]₂, —NRC(O)R, —NRC(O)OR, —S(O)—R, —S(O)OR, and —S(O)NRR;

R₂ and R₃ are independently hydrogen, (C₁₋₁₀)alkyl, (C₄₋₁₀)branched alkyl, (C₃₋₈)cycloalkyl; (C₃₋₈)cycloalkyl having at least one substituent selected from (C₁₋₁₀)alkyl, (C₄₋₁₀)branched alkyl, —O—(C₁-C₁₀)alkyl, —S—(C₁-C₁₀)alkyl, —NRR, —NO₂, —CN, —C(O)R, —C(S)R, —C(O)OR, —C(S)OR, —SC(S)R, —OC(S)R, —C(O)NRR, —C(S)NRR, —C(O)NR(OR), —C(S)NR(OR), —C(O)NR(SR), —C(S)NR(SR), —CH(CN)₂, —CH[C(O)R]₂, —CH[C(S)R]₂, —CH[C(O)OR]₂, —CH[C(S)OR]₂, —CH[C(O)SR]₂, —CH[C(S)SR]₂, —NRC(O)R, —NRC(O)OR, —S(O)—R, —S(O)OR, and —S(O)NRR; (C₆₋₁₈)aryl; or (C₆₋₁₈)aryl having at least one substituent selected from (C₁₋₁₀)alkyl, (C₄₋₁₀)branched alkyl, —O—(C₁-C₁₀)alkyl, —S—(C₁-C₁₀)alkyl, —NRR, —NO₂, —CN, —C(O)R, —C(S)R, —C(O)OR, —C(S)OR, —SC(S)R, —OC(S)R, —C(O)NRR, —C(S)NRR, —C(O)NR(OR), —C(S)NR(OR), —C(O)NR(SR), —C(S)NR(SR), —CH(CN)₂, —CH[C(O)R]₂, —CH[C(S)R]₂, —CH[C(O)OR]₂, —CH[C(S)OR]₂, —CH[C(O)SR]₂, —CH[C(S)SR]₂, —NRC(O)R, —NRC(O)OR, —S(O)—R, —S(O)OR, and —S(O)NRR; or

R₂ and R₃ are linked together to form a nitrogen containing ring system, wherein the nitrogen containing ring system is optionally substituted by at least one substituent selected from (C₁₋₁₀)alkyl, (C₄₋₁₀)branched alkyl, —O—(C₁-C₁₀)alkyl, —S—(C₁-C₁₀)alkyl, —NRR, —NO₂, —CN, —C(O)R, —C(S)R, —C(O)OR, —C(S)OR, —SC(S)R, —OC(S)R, —C(O)NRR, —C(S)NRR, —C(O)NR(OR), —C(S)NR(OR), —C(O)NR(SR), —C(S)NR(SR), —CH(CN)₂, —CH[C(O)R]₂, —CH[C(S)R]₂, —CH[C(O)OR]₂, —CH[C(S)OR]₂, —CH[C(O)SR]₂, —CH[C(S)SR]₂, —NRC(O)R, —NRC(O)OR, —S(O)—R, —S(O)OR, and —S(O)NRR;
wherein:

A is optionally substituted with one or more substituents selected from the group of (C₁-C₁₅)alkyl, (C₂-C₁₅)alkenyl, (C₂-C₁₅)alkynyl, —O—(C₁-C₁₅)alkyl, —O—(C₂-C₁₅) alkenyl, —O—(C₂-C₁₅) alkynyl, —S—(C₁-C₁₅)alkyl, —S—(C₂-C₁₅)alkenyl, —S—(C₂-C₁₅)alkynyl, (C₃-C₈)cycloalkyl, —O—(C₃—C₈)cycloalkyl, —S—(C₃-C₈)cycloalkyl, -halo, —NRR, —NO₂, —CN, —C(O)R, —C(S)R, —C(O)OR, —C(S)OR, —SC(S)R, —OC(S)R, —C(O)NRR, —C(S)NRR, —C(O)NR(OR), —C(S)NR(OR), —C(O)NR(SR), —C(S)NR(SR), —CH(CN)₂, —CH[C(O)R]₂, —CH[C(S)R]₂, —CH[C(O)OR]₂, —CH[C(S)OR]₂, —CH[C(O)SR]₂, —CH[C(S)SR]₂, —NRC(O)R, —NRC(O)OR, —S(O)—R, —S(O)OR, and —S(O)NRR;
wherein:

each R is independently selected from —H, (C₁-C₁₀)alkyl, (C₂-C₁₀)alkenyl, (C₂-C₁₀)alkynyl, (C₃-C₈)cycloalkyl, aryl or substituted aryl;

or a pharmaceutically acceptable salt, a prodrug, a tautomer or a solvate thereof.

In an embodiment, the present disclosure relates to a compound of Formula III:

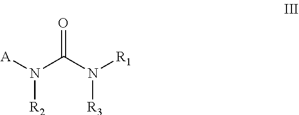

wherein:

A is an arene or a heteroarene;

R₁ is a (C₄₋₁₅)-branched alkyl; (C₃₋₈)cycloalkyl; (C₃₋₈)cycloalkyl having at least one substituent selected from (C₁₋₁₀)alkyl, (C₄₋₁₀)branched alkyl, —O—(C₁-C₁₀)alkyl, —S—(C₁-C₁₀)alkyl, —NRR, —NO₂, —CN, —C(O)R, —C(S)R, —C(O)OR, —C(S)OR, —SC(S)R, —OC(S)R, —C(O)NRR, —C(S)NRR, —C(O)NR(OR), —C(S)NR(OR), —C(O)NR(SR), —C(S)NR(SR), —CH(CN)₂, —CH[C(O)R]₂, —CH[C(S)R]₂, —CH[C(O)OR]₂, —CH[C(S)OR]₂, —CH[C(O)SR]₂, —CH[C(S)SR]₂, —NRC(O)R, —NRC(O)OR, —S(O)—R, —S(O)OR, and —S(O)NRR; (C₆₋₁₈)aryl; or (C₆₋₁₈)aryl having at least one substituent selected from (C₁₋₁₀)alkyl, (C₄₋₁₀)branched alkyl, —O—(C₁-C₁₀)alkyl, —S—(C₁-C₁₀)alkyl, —NRR, —NO₂, —CN, —C(O)R, —C(S)R, —C(O)OR, —C(S)OR, —SC(S)R, —OC(S)R, —C(O)NRR, —C(S)NRR, —C(O)NR(OR), —C(S)NR(OR), —C(O)NR(SR), —C(S)NR(SR), —CH(CN)₂, —CH[C(O)R]₂, —CH[C(S)R]₂, —CH[C(O)OR]₂, —CH[C(S)OR]₂, —CH[C(O)SR]₂, —CH[C(S)SR]₂, —NRC(O)R, —NRC(O)OR, —S(O)—R, —S(O)OR, and —S(O)NRR;

R₂ and R₃ are independently hydrogen, (C₁₋₁₀)alkyl, (C₄₋₁₀)branched alkyl, (C₃₋₈)cycloalkyl; (C₃₋₈)cycloalkyl having at least one substituent selected from (C₁₋₁₀)alkyl, (C₄₋₁₀)branched alkyl, —O—(C₁-C₁₀)alkyl, —S—(C₁-C₁₀)alkyl, —NRR, —NO₂, —CN, —C(O)R, —C(S)R, —C(O)OR, —C(S)OR, —SC(S)R, —OC(S)R, —C(O)NRR, —C(S)NRR, —C(O)NR(OR), —C(S)NR(OR), —C(O)NR(SR), —C(S)NR(SR), —CH(CN)₂, —CH[C(O)R]₂, —CH[C(S)R]₂, —CH[C(O)OR]₂, —CH[C(S)OR]₂, —CH[C(O)SR]₂, —CH[C(S)SR]₂, —NRC(O)R, —NRC(O)OR, —S(O)—R, —S(O)OR, and —S(O)NRR; (C₆₋₁₈)aryl; or (C₆₋₁₈)aryl having at least one substituent selected from (C₁₋₁₀)alkyl, (C₄₋₁₀)branched alkyl, —O—(C₁-C₁₀)alkyl, —S—(C₁-C₁₀)alkyl, —NRR, —NO₂, —CN, —C(O)R, —C(S)R, —C(O)OR, —C(S)OR, —SC(S)R, —OC(S)R, —C(O)NRR, —C(S)NRR, —C(O)NR(OR), —C(S)NR(OR), —C(O)NR(SR), —C(S)NR(SR), —CH(CN)₂, —CH[C(O)R]₂, —CH[C(S)R]₂, —CH[C(O)OR]₂, —CH[C(S)OR]₂, —CH[C(O)SR]₂, —CH[C(S)SR]₂, —NRC(O)R, —NRC(O)OR, —S(O)—R, —S(O)OR, and —S(O)NRR; or

R₂ and R₃ are linked together to form a nitrogen containing ring system, wherein the nitrogen containing ring system is optionally substituted by at least one substituent selected from (C₁₋₁₀)alkyl, (C₄₋₁₀)branched alkyl, —O—(C₁-C₁₀)alkyl, —S—(C₁-C₁₀)alkyl, —NRR, —NO₂, —CN, —C(O)R, —C(S)R, —C(O)OR, —C(S)OR, —SC(S)R, —OC(S)R, —C(O)NRR, —C(S)NRR, —C(O)NR(OR), —C(S)NR(OR), —C(O)NR(SR), —C(S)NR(SR), —CH(CN)₂, —CH[C(O)R]₂, —CH[C(S)R]₂, —CH[C(O)OR]₂, —CH[C(S)OR]₂, —CH[C(O)SR]₂, —CH[C(S)SR]₂, —NRC(O)R, —NRC(O)OR, —S(O)—R, —S(O)OR, and —S(O)NRR;
wherein:

A is optionally substituted with one or more substituents selected from the group of (C₁-C₁₅)alkyl, (C₂-C₁₅)alkenyl, (C₂-C₁₅)alkynyl, —O—(C₁-C₁₅)alkyl, —O—(C₂-C₁₅) alkenyl, —O—(C₂-C₁₅) alkynyl, —S—(C₁-C₁₅)alkyl, —S—(C₂-C₁₅)alkenyl, —S—(C₂-C₁₅)alkynyl, (C₃-C₈)cycloalkyl, —O—(C₃-C₈)cycloalkyl, —S—(C₃-C₈)cycloalkyl, -halo, —NRR, —NO₂, —CN, —C(O)R, —C(S)R, —C(O)OR, —C(S)OR, —SC(S)R, —OC(S)R, —C(O)NRR, —C(S)NRR, —C(O)NR(OR), —C(S)NR(OR), —C(O)NR(SR), —C(S)NR(SR), —CH(CN)₂, —CH[C(O)R]₂, —CH[C(S)R]₂, —CH[C(O)OR]₂, —CH[C(S)OR]₂, —CH[C(O)SR]₂, —CH[C(S)SR]₂, —NRC(O)R, —NRC(O)OR, —S(O)—R, —S(O)OR, and —S(O)NRR;
wherein:
   each R is independently selected from —H, (C₁-C₁₀)alkyl, (C₂-C₁₀)alkenyl, (C₂-C₁₀)alkynyl, (C₃-C₈)cycloalkyl, aryl or substituted aryl;

or a pharmaceutically acceptable salt, a prodrug, a tautomer or a solvate thereof;

for use as an anti-proliferative agent in the attenuation, inhibition, or prevention of conditions associated with IL-6 expression.

In an embodiment, the present disclosure relates to a compound of Formula III:

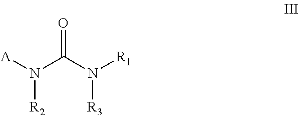

wherein:

A is an arene or a heteroarene;

R₁ is a (C₄₋₁₅)-branched alkyl; (C₃₋₈)cycloalkyl; (C₃₋₈)cycloalkyl having at least one substituent selected from (C₁₋₁₀)alkyl, (C₄₋₁₀)branched alkyl, —O—(C₁-C₁₀)alkyl, —S—(C₁-C₁₀)alkyl, —NRR, —NO₂, —CN, —C(O)R, —C(S)R, —C(O)OR, —C(S)OR, —SC(S)R, —OC(S)R, —C(O)NRR, —C(S)NRR, —C(O)NR(OR), —C(S)NR(OR), —C(O)NR(SR), —C(S)NR(SR), —CH(CN)₂, —CH[C(O)R]₂, —CH[C(S)R]₂, —CH[C(O)OR]₂, —CH[C(S)OR]₂, —CH[C(O)SR]₂, —CH[C(S)SR]₂, —NRC(O)R, —NRC(O)OR, —S(O)—R, —S(O)OR, and —S(O)NRR; (C₆₋₁₈)aryl; or (C₆₋₁₈)aryl having at least one substituent selected from (C₁₋₁₀)alkyl, (C₄₋₁₀)branched alkyl, —O—(C₁-C₁₀)alkyl, —S—(C₁-C₁₀)alkyl, —NRR, —NO₂, —CN, —C(O)R, —C(S)R, —C(O)OR, —C(S)OR, —SC(S)R, —OC(S)R, —C(O)NRR, —C(S)NRR, —C(O)NR(OR), —C(S)NR(OR), —C(O)NR(SR), —C(S)NR(SR), —CH(CN)₂, —CH[C(O)R]₂, —CH[C(S)R]₂, —CH[C(O)OR]₂, —CH[C(S)OR]₂, —CH[C(O)SR]₂, —CH[C(S)SR]₂, —NRC(O)R, —NRC(O)OR, —S(O)—R, —S(O)OR, and —S(O)NRR;

R₂ and R₃ are independently hydrogen, (C₁₋₁₀)alkyl, (C₄₋₁₀)branched alkyl, (C₃₋₈)cycloalkyl; (C₃₋₈)cycloalkyl having at least one substituent selected from (C₁₋₁₀)alkyl, (C₄₋₁₀)branched alkyl, —O—(C₁-C₁₀)alkyl, —S—(C₁-C₁₀)alkyl, —NRR, —NO₂, —CN, —C(O)R, —C(S)R, —C(O)OR, —C(S)OR, —SC(S)R, —OC(S)R, —C(O)NRR, —C(S)NRR, —C(O)NR(OR), —C(S)NR(OR), —C(O)NR(SR), —C(S)NR(SR), —CH(CN)₂, —CH[C(O)R]₂, —CH[C(S)R]₂, —CH[C(O)OR]₂, —CH[C(S)OR]₂, —CH[C(O)SR]₂, —CH[C(S)SR]₂, —NRC(O)R, —NRC(O)OR, —S(O)—R, —S(O)OR, and —S(O)NRR; (C₆₋₁₈)aryl; or (C₆₋₁₈)aryl having at least one substituent selected from (C₁₋₁₀)alkyl, (C₄₋₁₀)branched alkyl, —O—(C₁-C₁₀)alkyl, —S—(C₁-C₁₀)alkyl, —NRR, —NO₂, —CN, —C(O)R, —C(S)R, —C(O)OR, —C(S)OR, —SC(S)R, —OC(S)R, —C(O)NRR, —C(S)NRR, —C(O)NR(OR), —C(S)NR(OR), —C(O)NR(SR), —C(S)NR(SR), —CH(CN)₂, —CH[C(O)R]₂, —CH[C(S)R]₂, —CH[C(O)OR]₂, —CH[C(S)OR]₂, —CH[C(O)SR]₂, —CH[C(S)SR]₂, —NRC(O)R, —NRC(O)OR, —S(O)—R, —S(O)OR, and —S(O)NRR; or

R₂ and R₃ are linked together to form a nitrogen containing ring system, wherein the nitrogen containing ring system is optionally substituted by at least one substituent selected from (C₁₋₁₀)alkyl, (C₄₋₁₀)branched alkyl, —O—(C₁-C₁₀)alkyl, —S—(C₁-C₁₀)alkyl, —NRR, —NO₂, —CN, —C(O)R, —C(S)R, —C(O)OR, —C(S)OR, —SC(S)R, —OC(S)R, —C(O)NRR, —C(S)NRR, —C(O)NR(OR), —C(S)NR(OR), —C(O)NR(SR), —C(S)NR(SR), —CH(CN)₂, —CH[C(O)R]₂, —CH[C(S)R]₂, —CH[C(O)OR]₂, —CH[C(S)OR]₂, —CH[C(O)SR]₂, —CH[C(S)SR]₂, —NRC(O)R, —NRC(O)OR, —S(O)—R, —S(O)OR, and —S(O)NRR;
wherein:

A is optionally substituted with one or more substituents selected from the group of (C₁-C₁₅)alkyl, (C₂-C₁₅)alkenyl, (C₂-C₁₅)alkynyl, —O—(C₁-C₁₅)alkyl, —O—(C₂-C₁₅) alkenyl, —O—(C₂-C₁₅) alkynyl, —S—(C₁-C₁₅)alkyl, —S—(C₂-C₁₅)alkenyl, —S—(C₂-C₁₅)alkynyl, (C₃-C₈)cycloalkyl, —O—(C₃-C₈)cycloalkyl, —S—(C₃-C₈)cycloalkyl, -halo, —NRR, —NO₂, —CN, —C(O)R, —C(S)R, —C(O)OR, —C(S)OR, —SC(S)R, —OC(S)R, —C(O)NRR, —C(S)NRR, —C(O)NR(OR), —C(S)NR(OR), —C(O)NR(SR), —C(S)NR(SR), —CH(CN)₂, —CH[C(O)R]₂, —CH[C(S)R]₂, —CH[C(O)OR]₂, —CH[C(S)OR]₂, —CH[C(O)SR]₂, —CH[C(S)SR]₂, —NRC(O)R, —NRC(O)OR, —S(O)—R, —S(O)OR, and —S(O)NRR;
wherein:
   each R is independently selected from —H, (C₁-C₁₀)alkyl, (C₂-C₁₀)alkenyl, (C₂-C₁₀)alkynyl, (C₃-C₈)cycloalkyl, aryl or substituted aryl;

or a pharmaceutically acceptable salt, a prodrug, a tautomer or a solvate thereof;

for use as a therapeutic agent in the attenuation, inhibition, or prevention of conditions associated with IL-6 expression.

In an embodiment, the present disclosure relates to a method for treating, attenuating, inhibiting, or preventing a condition associated with IL-6 expression in a subject in need thereof, the method comprising administering a therapeutically effective amount of a compound of Formula III:

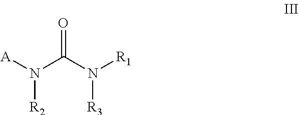

wherein:

A is an arene or a heteroarene;

R₁ is a (C₄₋₁₅)-branched alkyl; (C₃₋₈)cycloalkyl; (C₃₋₈)cycloalkyl having at least one substituent selected from (C₁₋₁₀)alkyl, (C₄₋₁₀)branched alkyl, —O—(C₁-C₁₀)alkyl, —S—(C₁-C₁₀)alkyl, —NRR, —NO₂, —CN, —C(O)R, —C(S)R, —C(O)OR, —C(S)OR, —SC(S)R, —OC(S)R, —C(O)NRR, —C(S)NRR, —C(O)NR(OR), —C(S)NR(OR), —C(O)NR(SR), —C(S)NR(SR), —CH(CN)₂, —CH[C(O)R]₂, —CH[C(S)R]₂, —CH[C(O)OR]₂, —CH[C(S)OR]₂, —CH[C(O)SR]₂, —CH[C(S)SR]₂, —NRC(O)R, —NRC(O)OR, —S(O)—R, —S(O)OR, and —S(O)NRR; (C₆₋₁₈)aryl; or (C₆₋₁₈)aryl having at least one substituent selected from (C₁₋₁₀)alkyl, (C₄₋₁₀)branched alkyl, —O—(C₁-C₁₀)alkyl, —S—(C₁-C₁₀)alkyl, —NRR, —NO₂, —CN, —C(O)R, —C(S)R, —C(O)OR, —C(S)OR, —SC(S)R, —OC(S)R, —C(O)NRR, —C(S)NRR, —C(O)NR(OR), —C(S)NR(OR), —C(O)NR(SR), —C(S)NR(SR), —CH(CN)₂, —CH[C(O)R]₂, —CH[C(S)R]₂, —CH[C(O)OR]₂, —CH[C(S)OR]₂, —CH[C(O)SR]₂, —CH[C(S)SR]₂, —NRC(O)R, —NRC(O)OR, —S(O)—R, —S(O)OR, and —S(O)NRR;

R₂ and R₃ are independently hydrogen, (C₁₋₁₀)alkyl, (C₄₋₁₀)branched alkyl, (C₃₋₈)cycloalkyl; (C₃₋₈)cycloalkyl having at least one substituent selected from (C₁₋₁₀)alkyl, (C₄₋₁₀)branched alkyl, —O—(C₁-C₁₀)alkyl, —S—(C₁-C₁₀)alkyl, —NRR, —NO₂, —CN, —C(O)R, —C(S)R, —C(O)OR, —C(S)OR, —SC(S)R, —OC(S)R, —C(O)NRR, —C(S)NRR, —C(O)NR(OR), —C(S)NR(OR), —C(O)NR(SR), —C(S)NR(SR), —CH(CN)₂, —CH[C(O)R]₂, —CH[C(S)R]₂, —CH[C(O)OR]₂, —CH[C(S)OR]₂, —CH[C(O)SR]₂, —CH[C(S)SR]₂, —NRC(O)R, —NRC(O)OR, —S(O)—R, —S(O)OR, and —S(O)NRR; (C₆₋₁₈)aryl; or (C₆₋₁₈)aryl having at least one substituent selected from (C₁₋₁₀)alkyl, (C₄₋₁₀)branched alkyl, —O—(C₁-C₁₀)alkyl, —S—(C₁-C₁₀)alkyl, —NRR, —NO₂, —CN, —C(O)R, —C(S)R, —C(O)OR, —C(S)OR, —SC(S)R, —OC(S)R, —C(O)NRR, —C(S)NRR, —C(O)NR(OR), —C(S)NR(OR), —C(O)NR(SR), —C(S)NR(SR), —CH(CN)₂, —CH[C(O)R]₂, —CH[C(S)R]₂, —CH[C(O)OR]₂, —CH[C(S)OR]₂, —CH[C(O)SR]₂, —CH[C(S)SR]₂, —NRC(O)R, —NRC(O)OR, —S(O)—R, —S(O)OR, and —S(O)NRR; or

R₂ and R₃ are linked together to form a nitrogen containing ring system, wherein the nitrogen containing ring system is optionally substituted by at least one substituent selected from (C₁₋₁₀)alkyl, (C₄₋₁₀)branched alkyl, —O—(C₁-C₁₀)alkyl, —S—(C₁-C₁₀)alkyl, —NRR, —NO₂, —CN, —C(O)R, —C(S)R, —C(O)OR, —C(S)OR, —SC(S)R, —OC(S)R, —C(O)NRR, —C(S)NRR, —C(O)NR(OR), —C(S)NR(OR), —C(O)NR(SR), —C(S)NR(SR), —CH(CN)₂, —CH[C(O)R]₂, —CH[C(S)R]₂, —CH[C(O)OR]₂, —CH[C(S)OR]₂, —CH[C(O)SR]₂, —CH[C(S)SR]₂, —NRC(O)R, —NRC(O)OR, —S(O)—R, —S(O)OR, and —S(O)NRR;
wherein:

A is optionally substituted with one or more substituents selected from the group of (C₁-C₁₅)alkyl, (C₂-C₁₅)alkenyl, (C₂-C₁₅)alkynyl, —O—(C₁-C₁₅)alkyl, —O—(C₂-C₁₅) alkenyl, —O—(C₂-C₁₅) alkynyl, —S—(C₁-C₁₅)alkyl, —S—(C₂-C₁₅)alkenyl, —S—(C₂-C₁₅)alkynyl, (C₃-C₈)cycloalkyl, —O—(C₃-C₈)cycloalkyl, —S—(C₃-C₈)cycloalkyl, -halo, —NRR, —NO₂, —CN, —C(O)R, —C(S)R, —C(O)OR, —C(S)OR, —SC(S)R, —OC(S)R, —C(O)NRR, —C(S)NRR, —C(O)NR(OR), —C(S)NR(OR), —C(O)NR(SR), —C(S)NR(SR), —CH(CN)₂, —CH[C(O)R]₂, —CH[C(S)R]₂, —CH[C(O)OR]₂, —CH[C(S)OR]₂, —CH[C(O)SR]₂, —CH[C(S)SR]₂, —NRC(O)R, —NRC(O)OR, —S(O)—R, —S(O)OR, and —S(O)NRR;
wherein:

each R is independently selected from —H, (C₁-C₁₀)alkyl, (C₂-C₁₀)alkenyl, (C₂-C₁₀)alkynyl, (C₃-C₈)cycloalkyl, aryl or substituted aryl;

or a pharmaceutically acceptable salt, a prodrug, a tautomer or a solvate thereof.

In an embodiment, the present disclosure relates to a pharmaceutical composition comprising a compound of Formula III and a pharmaceutically acceptable carrier.

In an embodiment, the present disclosure relates to a medicament for use in treating, attenuating, inhibiting and/or preventing inflammation and inflammation-related pathologies, the medicament comprising a compound of Formula III and a pharmaceutically acceptable carrier.

In an embodiment, the present disclosure relates to a medicament for use in treating a condition associated with IL-6 expression, the medicament comprising a compound of Formula III and a pharmaceutically acceptable carrier.

In an embodiment, the present disclosure relates to a compound of Formula III:

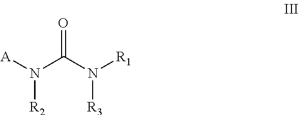

wherein:

A is an arene;

R₁ is a (C₄₋₁₅)-branched alkyl; (C₃₋₈)cycloalkyl; or (C₃₋₈)cycloalkyl having at least one substituent selected from (C₁₋₁₀)alkyl, (C₄₋₁₀)branched alkyl, —O—(C₁-C₁₀)alkyl, —S—(C₁-C₁₀)alkyl, —NRR, —CN, —C(O)R, —C(O)OR, —C(O)NRR, —NRC(O)R, —S(O)—R, —S(O)OR, and —S(O)NRR;

R₂ and R₃ are independently hydrogen, (C₁₋₁₀)alkyl, (C₄₋₁₀)branched alkyl, (C₃₋₈)cycloalkyl; (C₃₋₈)cycloalkyl having at least one substituent selected from (C₁₋₁₀)alkyl, (C₄₋₁₀)branched alkyl, —O—(C₁-C₁₀)alkyl, —S—(C₁-C₁₀)alkyl, —NRR, —CN, —C(O)R, —C(O)OR, —C(O)NRR, —S(O)—R, —S(O)OR, and —S(O)NRR; or

R₂ and R₃ are linked together to form a nitrogen containing ring system, wherein the nitrogen containing ring system is optionally substituted by at least one substituent selected from (C₁₋₁₀)alkyl, (C₄₋₁₀)branched alkyl, —O—(C₁-C₁₀)alkyl, —S—(C₁-C₁₀)alkyl, —NRR, CN, —C(O)R, —C(O)OR, —C(O)NRR, —S(O)—R, —S(O)OR, and —S(O)NRR;
wherein:

A is optionally substituted with one or more substituents selected from the group of (C₁-C₁₅)alkyl, —O—(C₁-C₁₅)alkyl, —S—(C₁-C₁₅)alkyl, (C₃-C₈)cycloalkyl, —O—(C₃-C₈)cycloalkyl, —S—(C₃-C₈)cycloalkyl, -halo, —NRR, —CN, —C(O)R, —C(O)OR, —C(O)NRR, —NRC(O)R, —NRC(O)OR, —S(O)—R, —S(O)OR, and —S(O)NRR;
wherein:

each R is independently selected from —H, (C₁-C₁₀)alkyl,

or a pharmaceutically acceptable salt, a prodrug, a tautomer or a solvate thereof.

In an embodiment, the present disclosure relates to a compound of Formula IV:

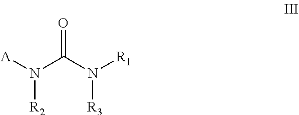

wherein:

A is an arene;

R₁ is a (C₄₋₁₅)-branched alkyl; (C₃₋₈)cycloalkyl; or (C₃₋₈)cycloalkyl having at least one substituent selected from (C₁₋₁₀)alkyl, (C₄₋₁₀)branched alkyl, —O—(C₁-C₁₀)alkyl, —S—(C₁-C₁₀)alkyl, —NRR, —CN, —C(O)R, —C(O)OR, —C(O)NRR, —NRC(O)R, —S(O)—R, —S(O)OR, and —S(O)NRR;

R₂ and R₃ are independently hydrogen, (C₁₋₁₀)alkyl, (C₄₋₁₀)branched alkyl, (C₃₋₈)cycloalkyl; (C₃₋₈)cycloalkyl having at least one substituent selected from (C₁₋₁₀)alkyl, (C₄₋₁₀)branched alkyl, —O—(C₁-C₁₀)alkyl, —S—(C₁-C₁₀)alkyl, —NRR, —CN, —C(O)R, —C(O)OR, —C(O)NRR, —S(O)—R, —S(O)OR, and —S(O)NRR; or

R₂ and R₃ are linked together to form a nitrogen containing ring system, wherein the nitrogen containing ring system is optionally substituted by at least one substituent selected from (C₁₋₁₀)alkyl, (C₄₋₁₀)branched alkyl, —O—(C₁-C₁₀)alkyl, —S—(C₁-C₁₀)alkyl, —NRR, CN, —C(O)R, —C(O)OR, —C(O)NRR, —S(O)—R, —S(O)OR, and —S(O)NRR;
wherein:

A is optionally substituted with one or more substituents selected from the group of (C₁-C₁₅)alkyl, —O—(C₁-C₁₅)alkyl, —S—(C₁-C₁₅)alkyl, (C₃-C₈)cycloalkyl, —O—(C₃-C₈)cycloalkyl, —S—(C₃-C₈)cycloalkyl, -halo, —NRR, —CN, —C(O)R, —C(O)OR, —C(O)NRR, —NRC(O)R, —NRC(O)OR, —S(O)—R, —S(O)OR, and —S(O)NRR;
wherein:

each R is independently selected from —H, (C₁-C₁₀)alkyl,

or a pharmaceutically acceptable salt, a prodrug, a tautomer or a solvate thereof;

for use as an anti-proliferative agent in the attenuation, inhibition, or prevention of conditions associated with IL-6 expression.

In an embodiment, the present disclosure relates to a compound of Formula III:

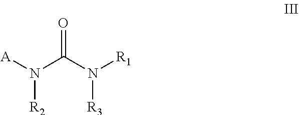

wherein:

A is an arene;

R₁ is a (C₄₋₁₅)-branched alkyl; (C₃₋₈)cycloalkyl; or (C₃₋₈)cycloalkyl having at least one substituent selected from (C₁₋₁₀)alkyl, (C₄₋₁₀)branched alkyl, —O—(C₁-C₁₀)alkyl, —S—(C₁-C₁₀)alkyl, —NRR, —CN, —C(O)R, —C(O)OR, —C(O)NRR, —NRC(O)R, —S(O)—R, —S(O)OR, and —S(O)NRR;

R₂ and R₃ are independently hydrogen, (C₁₋₁₀)alkyl, (C₄₋₁₀)branched alkyl, (C₃₋₈)cycloalkyl; (C₃₋₈)cycloalkyl having at least one substituent selected from (C₁₋₁₀)alkyl, (C₄₋₁₀)branched alkyl, —O—(C₁-C₁₀)alkyl, —S—(C₁-C₁₀)alkyl, —NRR, —CN, —C(O)R, —C(O)OR, —C(O)NRR, —S(O)—R, —S(O)OR, and —S(O)NRR; or

R₂ and R₃ are linked together to form a nitrogen containing ring system, wherein the nitrogen containing ring system is optionally substituted by at least one substituent selected from (C₁₋₁₀)alkyl, (C₄₋₁₀)branched alkyl, —O—(C₁-C₁₀)alkyl, —S—(C₁-C₁₀)alkyl, —NRR, CN, —C(O)R, —C(O)OR, —C(O)NRR, —S(O)—R, —S(O)OR, and —S(O)NRR;
wherein:

A is optionally substituted with one or more substituents selected from the group of (C₁-C₁₅)alkyl, —O—(C₁-C₁₅)alkyl, —S—(C₁-C₁₅)alkyl, (C₃-C₈)cycloalkyl, —O—(C₃-C₈)cycloalkyl, —S—(C₃-C₈)cycloalkyl, -halo, —NRR, —CN, —C(O)R, —C(O)OR, —C(O)NRR, —NRC(O)R, —NRC(O)OR, —S(O)—R, —S(O)OR, and —S(O)NRR;
wherein:

each R is independently selected from —H, (C₁-C₁₀)alkyl,

or a pharmaceutically acceptable salt, a prodrug, a tautomer or a solvate thereof;

for use as a therapeutic agent in the attenuation, inhibition, or prevention of conditions associated with IL-6 expression.

In an embodiment, the present disclosure relates to a method for treating, attenuating, inhibiting, or preventing a condition associated with IL-6 expression in a subject in need thereof, the method comprising administering a therapeutically effective amount of a compound of Formula III:

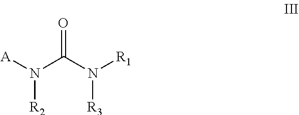

wherein:

A is an arene;

R₁ is a (C₄₋₁₅)-branched alkyl; (C₃₋₈)cycloalkyl; or (C₃₋₈)cycloalkyl having at least one substituent selected from (C₁₋₁₀)alkyl, (C₄₋₁₀)branched alkyl, —O—(C₁-C₁₀)alkyl, —S—(C₁-C₁₀)alkyl, —NRR, —CN, —C(O)R, —C(O)OR, —C(O)NRR, —NRC(O)R, —S(O)—R, —S(O)OR, and —S(O)NRR;

R₂ and R₃ are independently hydrogen, (C₁₋₁₀)alkyl, (C₄₋₁₀)branched alkyl, (C₃₋₈)cycloalkyl; (C₃₋₈)cycloalkyl having at least one substituent selected from (C₁₋₁₀)alkyl, (C₄₋₁₀)branched alkyl, —O—(C₁-C₁₀)alkyl, —S—(C₁-C₁₀)alkyl, —NRR, —CN, —C(O)R, —C(O)OR, —C(O)NRR, —S(O)—R, —S(O)OR, and —S(O)NRR; or

R₂ and R₃ are linked together to form a nitrogen containing ring system, wherein the nitrogen containing ring system is optionally substituted by at least one substituent selected from (C₁₋₁₀)alkyl, (C₄₋₁₀)branched alkyl, —O—(C₁-C₁₀)alkyl, —S—(C₁-C₁₀)alkyl, —NRR, CN, —C(O)R, —C(O)OR, —C(O)NRR, —S(O)—R, —S(O)OR, and —S(O)NRR;
wherein:

A is optionally substituted with one or more substituents selected from the group of (C₁-C₁₅)alkyl, —O—(C₁-C₁₅)alkyl, —S—(C₁-C₁₅)alkyl, (C₃-C₈)cycloalkyl, —O—(C₃-C₈)cycloalkyl, —S—(C₃-C₈)cycloalkyl, -halo, —NRR, —CN, —C(O)R, —C(O)OR, —C(O)NRR, —NRC(O)R, —NRC(O)OR, —S(O)—R, —S(O)OR, and —S(O)NRR;
wherein:

each R is independently selected from —H, (C₁-C₁₀)alkyl,

or a pharmaceutically acceptable salt, a prodrug, a tautomer or a solvate thereof.

In an embodiment, the present disclosure relates to a pharmaceutical composition comprising a compound of Formula III and a pharmaceutically acceptable carrier.

In an embodiment, the present disclosure relates to a medicament for use in treating, attenuating, inhibiting and/or preventing inflammation and inflammation-related pathologies, the medicament comprising a compound of Formula III and a pharmaceutically acceptable carrier.

In an embodiment, the present disclosure relates to a medicament for use in treating a condition associated with IL-6 expression, the medicament comprising a compound of Formula III and a pharmaceutically acceptable carrier.

In an embodiment, the present disclosure relates to a compound of Formula IV:

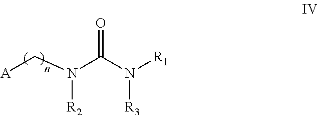

wherein:
   A is an arene;
   R₁ is a (C₄₋₁₅)alkyl; (C₄₋₁₅)-branched alkyl; (C₃₋₈)cycloalkyl; alk(C₃₋₈)cycloalkyl, (C₃₋₈)cycloalkenyl, alk(C₃₋₈)cycloalkenyl, (C₃₋₈)cycloalkyl having at least one substituent selected from (C₁₋₁₀)alkyl, (C₄₋₁₀)branched alkyl, —O—(C₁-C₁₀)alkyl, ROR—; —S—(C₁-C₁₀)alkyl, —NRR, —CN, —C(O)R, —C(O)OR, —C(O)NRR, —NRC(O)R, —S(O)—R, —S(O)OR, and —S(O)NRR; or (C₃₋₈)cycloalkenyl having at least one substituent selected from (C₁₋₁₀)alkyl, (C₄₋₁₀)branched alkyl, —O—(C₁-C₁₀)alkyl, ROR—; —S—(C₁-C₁₀)alkyl, —NRR, —CN, —C(O)R, —C(O)OR, —C(O)NRR, —NRC(O)R, —S(O)—R, —S(O)OR, and —S(O)NRR;
   R₂ and R₃ are independently hydrogen, (C₁₋₁₀)alkyl, (C₄₋₁₀)branched alkyl, (C₃₋₈)cycloalkyl; (C₃₋₈)cycloalkyl having at least one substituent selected from (C₁₋₁₀)alkyl, (C₄₋₁₀)branched alkyl, —O—(C₁-C₁₀)alkyl, —S—(C₁-C₁₀)alkyl, —NRR, —CN, —C(O)R, —C(O)OR, —C(O)NRR, —S(O)—R, —S(O)OR, and —S(O)NRR; or
   R₂ and R₃ are linked together to form a nitrogen containing ring system, wherein the nitrogen containing ring system is optionally substituted by at least one substituent selected from (C₁₋₁₀)alkyl, (C₄₋₁₀)branched alkyl, —O—(C₁-C₁₀)alkyl, —S—(C₁-C₁₀)alkyl, —NRR, CN, —C(O)R, —C(O)OR, —C(O)NRR, —S(O)—R, —S(O)OR, and —S(O)NRR;
n is 0, 1 or 2;
wherein:
   A is optionally substituted with one or more substituents selected from the group of (C₁-C₁₅)alkyl, —O—(C₁-C₁₅)alkyl, —S—(C₁-C₁₅)alkyl, (C₃-C₈)cycloalkyl, —O—(C₃-C₈)cycloalkyl, —S—(C₃-C₈)cycloalkyl, -halo, —NRR, —CN, —C(O)R, —C(O)OR, —C(O)NRR, —NRC(O)R, —NRC(O)OR, —S(O)—R, —S(O)OR, and —S(O)NRR;
wherein:
   each R is independently selected from —H, (C₁-C₁₀)alkyl,
   or a pharmaceutically acceptable salt, a prodrug, a tautomer or a solvate thereof.

In an embodiment, the present disclosure relates to a compound of Formula IV including:

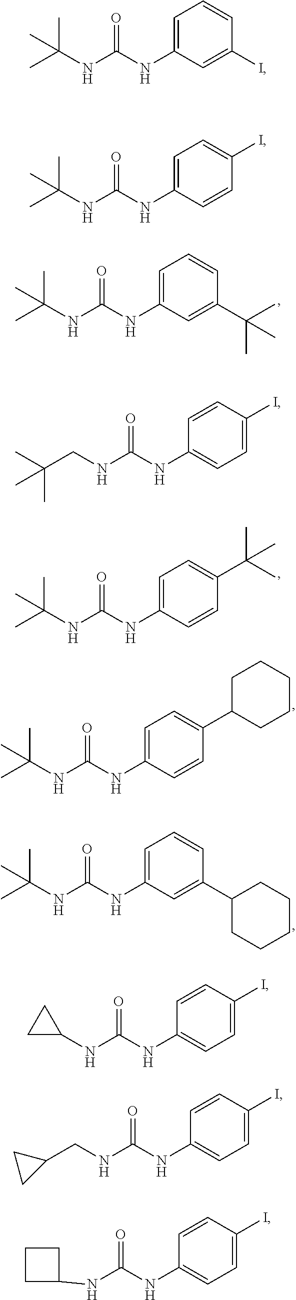

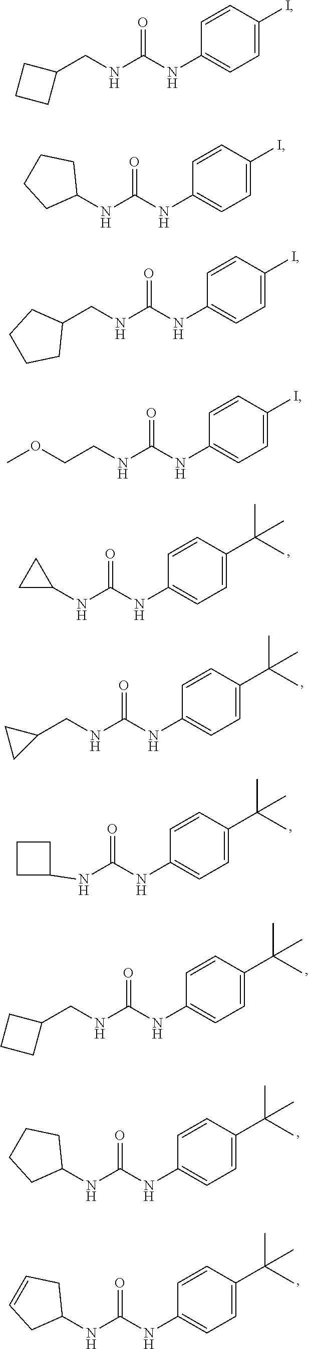

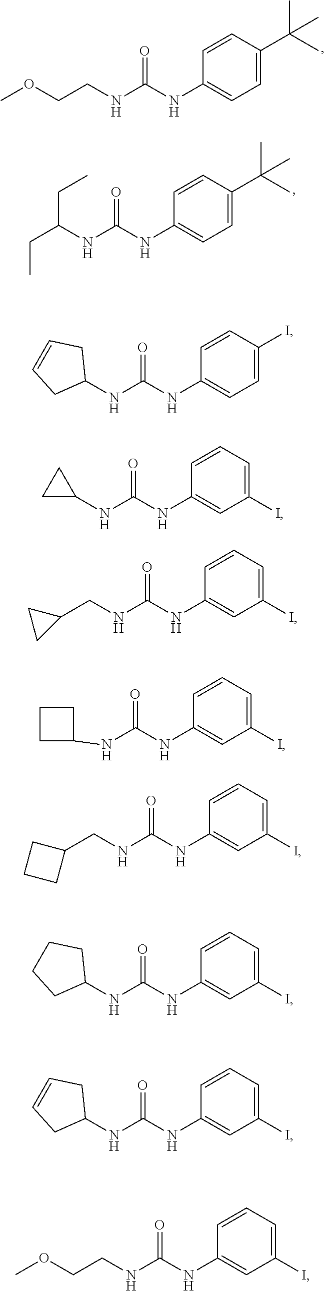

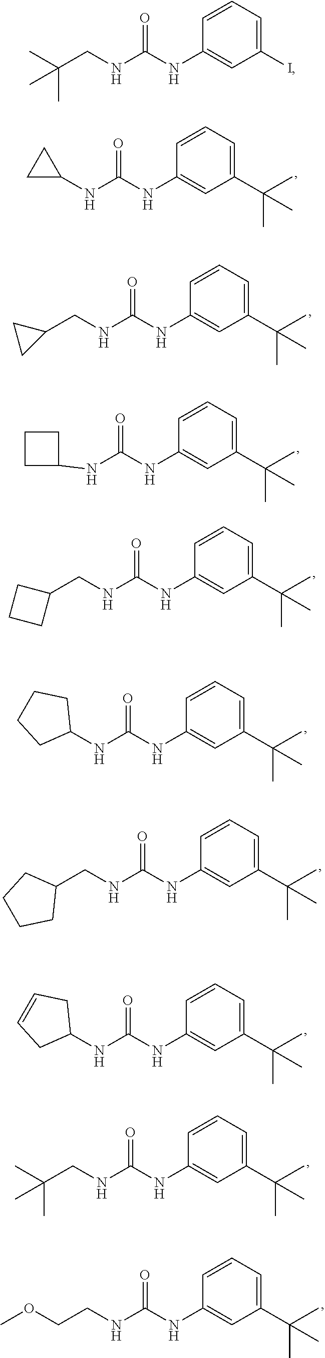

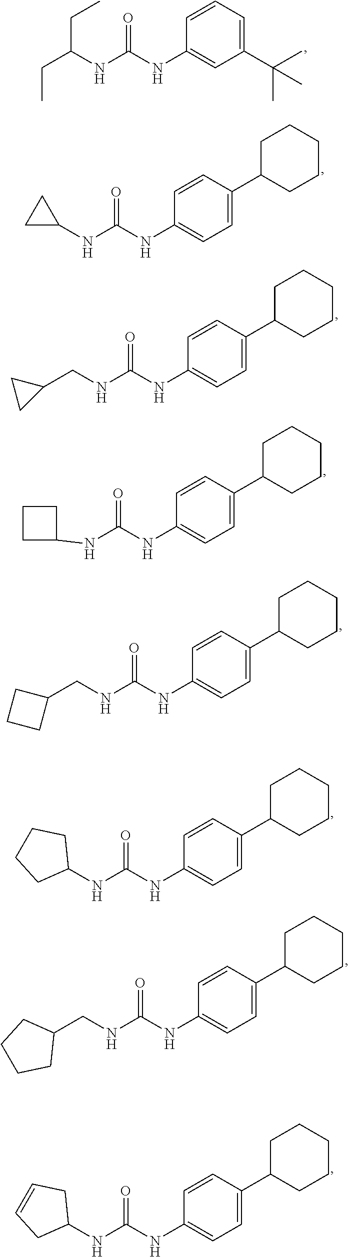

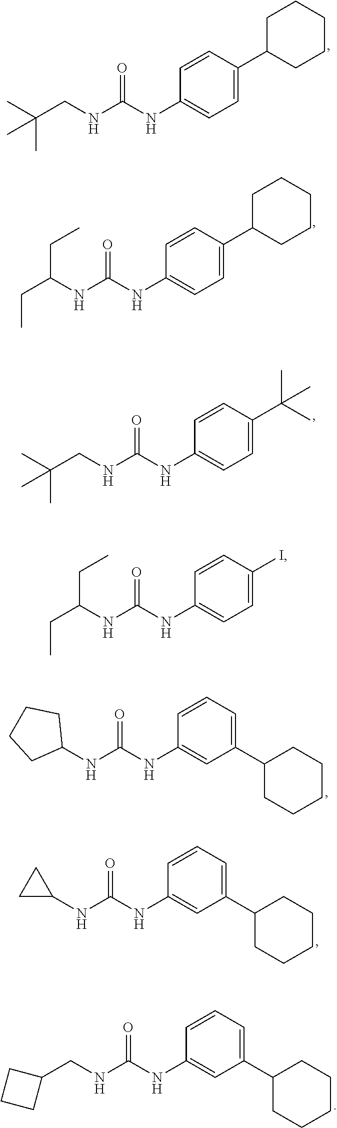

In an embodiment, the present disclosure relates to a pharmaceutical composition comprising a compound of Formula IV and a pharmaceutically acceptable carrier.

In an embodiment, the present disclosure relates to a medicament for use in treating, attenuating, inhibiting and/or preventing inflammation and inflammation-related pathologies, the medicament comprising a compound of Formula IV and a pharmaceutically acceptable carrier.

In an embodiment, the present disclosure relates to a medicament for use in treating a condition associated with IL-6 expression, the medicament comprising a compound of Formula IV and a pharmaceutically acceptable carrier.

In an embodiment, the present disclosure relates to a compound of Formula V:

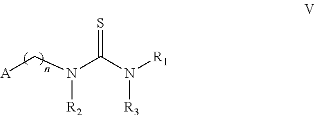

wherein:
   A is an arene;
   R₁ is a (C₁₋₁₅)alkyl; (C₄₋₁₅)-branched alkyl; (C₃₋₈)cycloalkyl; alk(C₃₋₈)cycloalkyl, (C₃₋₈)cycloalkenyl, alk(C₃₋₈)cycloalkenyl, (C₃₋₈)cycloalkyl having at least one substituent selected from (C₁₋₁₀)alkyl, (C₄₋₁₀)branched alkyl, —O—(C₁-C₁₀)alkyl, ROR—; —S—(C₁-C₁₀)alkyl, —NRR, —CN, —C(O)R, —C(O)OR, —C(O)NRR, —NRC(O)R, —S(O)—R, —S(O)OR, and —S(O)NRR; or (C₃₋₈)cycloalkenyl having at least one substituent selected from (C₁₋₁₀)alkyl, (C₄₋₁₀)branched alkyl, —O—(C₁-C₁₀)alkyl, ROR—; —S—(C₁-C₁₀)alkyl, —NRR, —CN, —C(O)R, —C(O)OR, —C(O)NRR, —NRC(O)R, —S(O)—R, —S(O)OR, and —S(O)NRR;
   R₂ and R₃ are independently hydrogen, (C₁₋₁₀)alkyl, (C₄₋₁₀)branched alkyl, (C₃₋₈)cycloalkyl; (C₃₋₈)cycloalkyl having at least one substituent selected from (C₁₋₁₀)alkyl, (C₄₋₁₀)branched alkyl, —O—(C₁-C₁₀)alkyl, —S—(C₁-C₁₀)alkyl, —NRR, —CN, —C(O)R, —C(O)OR, —C(O)NRR, —S(O)—R, —S(O)OR, and —S(O)NRR; or
   R₂ and R₃ are linked together to form a nitrogen containing ring system, wherein the nitrogen containing ring system is optionally substituted by at least one substituent selected from (C₁₋₁₀)alkyl, (C₄₋₁₀)branched alkyl, —O—(C₁-C₁₀)alkyl, —S—(C₁-C₁₀)alkyl, —NRR, CN, —C(O)R, —C(O)OR, —C(O)NRR, —S(O)—R, —S(O)OR, and —S(O)NRR;
n is 0, 1 or 2;
wherein:
   A is optionally substituted with one or more substituents selected from the group of (C₁-C₁₅)alkyl, —O—(C₁-C₁₅)alkyl, —S—(C₁-C₁₅)alkyl, (C₃-C₈)cycloalkyl, —O—(C₃-C₈)cycloalkyl, —S—(C₃-C₈)cycloalkyl, -halo, —NRR, —CN, —C(O)R, —C(O)OR, —C(O)NRR, —NRC(O)R, —NRC(O)OR, —S(O)—R, —S(O)OR, and —S(O)NRR;
wherein:
   each R is independently selected from —H, (C₁-C₁₀)alkyl,
   or a pharmaceutically acceptable salt, a prodrug, a tautomer or a solvate thereof.

In an embodiment, the present disclosure relates to a compound of Formula V including:

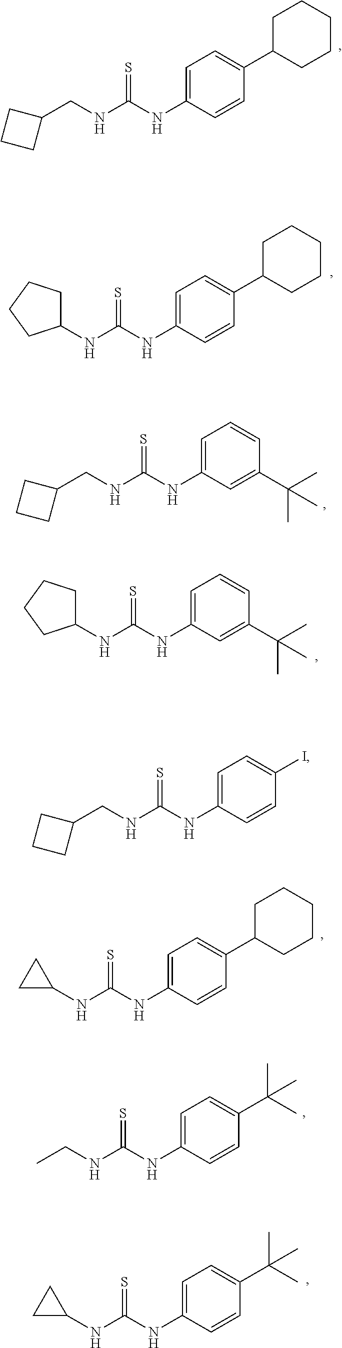

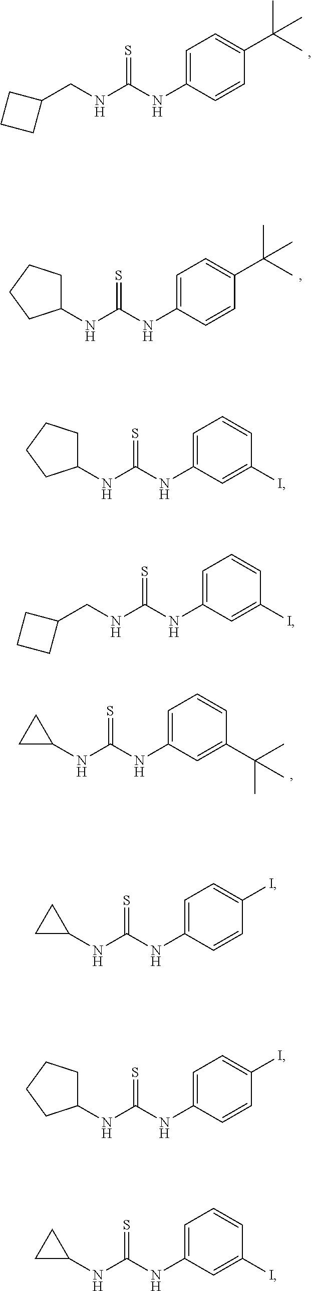

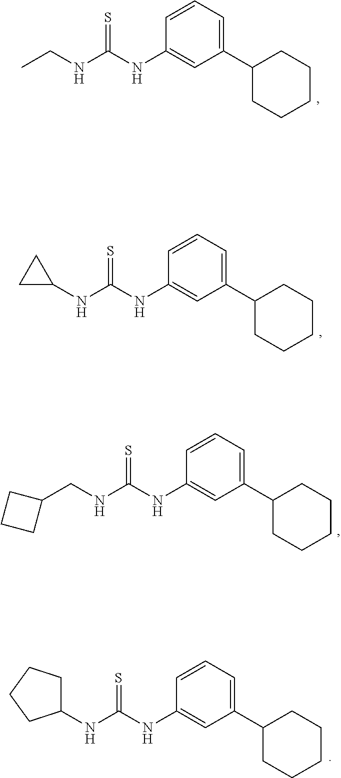 [SMILES: CCNC(=S)Nc1cccc(C2CCCCC2)c1 ; S=C(Nc1cccc(C2CCCCC2)c1)NC1CC1 ; S=C(NCC1CCC1)Nc1cccc(C2CCCCC2)c1 ; S=C(Nc1cccc(C2CCCCC2)c1)NC1CCCC1]

In an embodiment, the present disclosure relates to a pharmaceutical composition comprising a compound of Formula V and a pharmaceutically acceptable carrier.

In an embodiment, the present disclosure relates to a medicament for use in treating, attenuating, inhibiting and/or preventing inflammation and inflammation-related pathologies, the medicament comprising a compound of Formula V and a pharmaceutically acceptable carrier.

In an embodiment, the present disclosure relates to a medicament for use in treating a condition associated with IL-6 expression, the medicament comprising a compound of Formula V and a pharmaceutically acceptable carrier.

In an embodiment, the present disclosure relates to a compound of Formula VI:

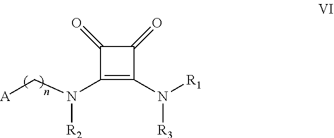

wherein:
   A is an arene;
   R₁ is a (C₁₋₁₅)alkyl; (C₄₋₁₅)-branched alkyl; (C₃₋₈)cycloalkyl; alk(C₃₋₈)cycloalkyl, (C₃₋₈)cycloalkenyl, alk(C₃₋₈)cycloalkenyl, (C₃₋₈)cycloalkyl having at least one substituent selected from (C₁₋₁₀)alkyl, (C₄₋₁₀)branched alkyl, —O—(C₁-C₁₀)alkyl, ROR—; —S—(C₁-C₁₀)alkyl, —NRR, —CN, —C(O)R, —C(O)OR, —C(O)NRR, —NRC(O)R, —S(O)—R, —S(O)OR, and —S(O)NRR; or (C₃₋₈)cycloalkenyl having at least one substituent selected from (C₁₋₁₀)alkyl, (C₄₋₁₀)branched alkyl, —O—(C₁-C₁₀)alkyl, ROR—; —S—(C₁-C₁₀)alkyl, —NRR, —CN, —C(O)R, —C(O)OR, —C(O)NRR, —NRC(O)R, —S(O)—R, —S(O)OR, and —S(O)NRR;
   R₂ and R₃ are independently hydrogen, (C₁₋₁₀)alkyl, (C₄₋₁₀)branched alkyl, (C₃₋₈)cycloalkyl; (C₃₋₈)cycloalkyl having at least one substituent selected from (C₁₋₁₀)alkyl, (C₄₋₁₀)branched alkyl, —O—(C₁-C₁₀)alkyl, —S—(C₁-C₁₀)alkyl, —NRR, —CN, —C(O)R, —C(O)OR, —C(O)NRR, —S(O)—R, —S(O)OR, and —S(O)NRR; or
   R₂ and R₃ are linked together to form a nitrogen containing ring system, wherein the nitrogen containing ring system is optionally substituted by at least one substituent selected from (C₁₋₁₀)alkyl, (C₄₋₁₀)branched alkyl, —O—(C₁-C₁₀)alkyl, —S—(C₁-C₁₀)alkyl, —NRR, CN, —C(O)R, —C(O)OR, —C(O)NRR, —S(O)—R, —S(O)OR, and —S(O)NRR;
n is 0, 1 or 2;
wherein:
   A is optionally substituted with one or more substituents selected from the group of (C₁-C₁₅)alkyl, —O—(C₁-C₁₅)alkyl, —S—(C₁-C₁₅)alkyl, (C₃-C₈)cycloalkyl, —O—(C₃-C₈)cycloalkyl, —S—(C₃-C₈)cycloalkyl, -halo, —NRR, —CN, —C(O)R, —C(O)OR, —C(O)NRR, —NRC(O)R, —NRC(O)OR, —S(O)—R, —S(O)OR, and —S(O)NRR;
wherein:
   each R is independently selected from —H, (C₁-C₁₀)alkyl,
   or a pharmaceutically acceptable salt, a prodrug, a tautomer or a solvate thereof.

In an embodiment, the present disclosure relates to a compound of Formula VI including:

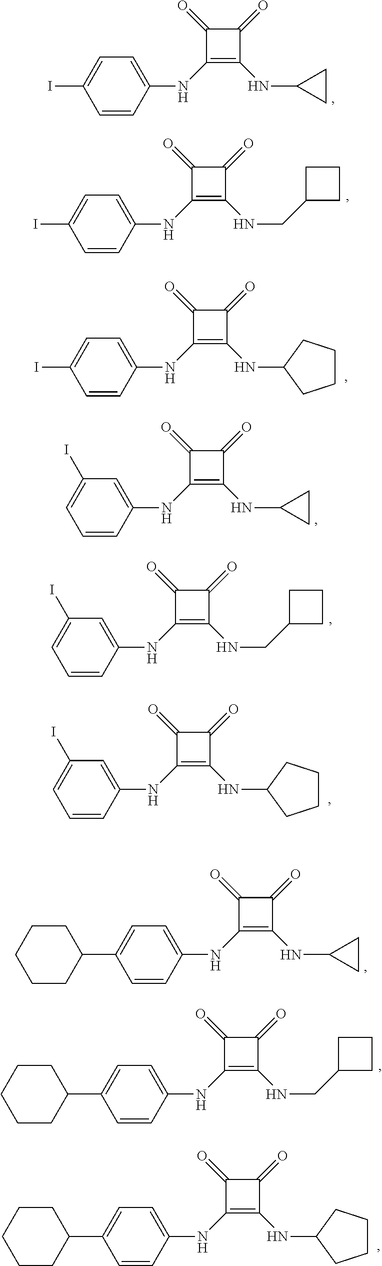

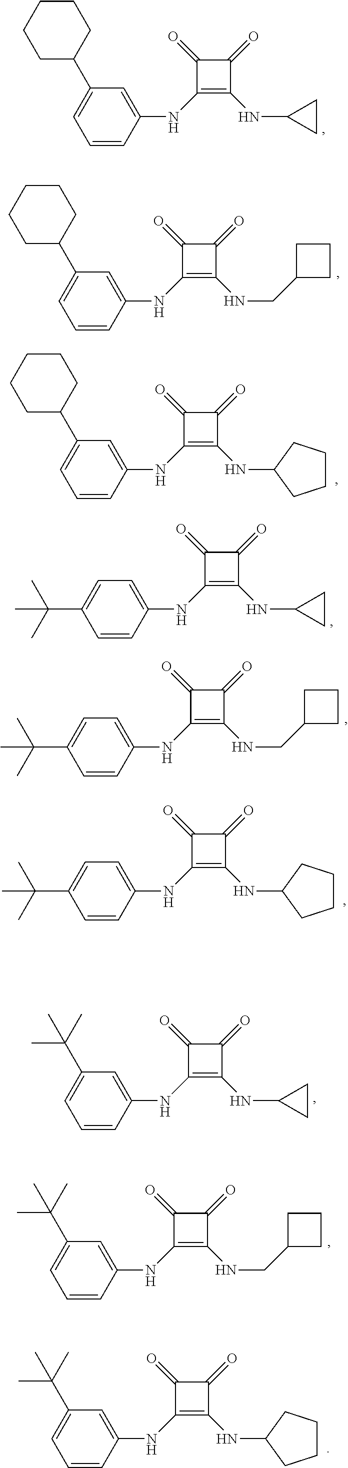

In an embodiment, the present disclosure relates to a pharmaceutical composition comprising a compound of Formula VI and a pharmaceutically acceptable carrier.

In an embodiment, the present disclosure relates to a medicament for use in treating, attenuating, inhibiting and/or preventing inflammation and inflammation-related pathologies, the medicament comprising a compound of Formula VI and a pharmaceutically acceptable carrier.

In an embodiment, the present disclosure relates to a medicament for use in treating a condition associated with IL-6 expression, the medicament comprising a compound of Formula VI and a pharmaceutically acceptable carrier.

The foregoing and other advantages and features of the present disclosure will become more apparent upon reading of the following non-restrictive description of illustrative embodiments thereof, given by way of example only with reference to the accompanying drawings/figures.

### BRIEF DESCRIPTION OF THE DRAWINGS/FIGURES

In the appended drawings/figures:

FIG. 1 is an illustration of the molecular structures of tBCEU and cHCEU respectively.

FIG. 2 is an illustration of the effect of compound 20 (Table 4) on the thickness of the epidermis in a mouse model of psoriasis; A) H&E staining of skin sections from mice topically treated for 6 days with base cream or B) imiquimod (5%) to induce psoriasis-like symptoms. The mice either received DMSO as a negative control, dexamethasone as a positive control or compound 20. The epidermis thickness was measured on 8 mice per condition (C).

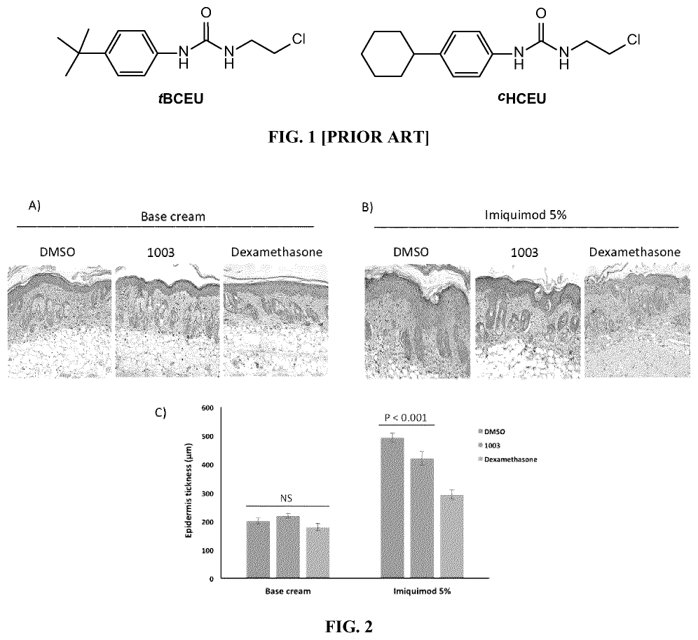

### DETAILED DESCRIPTION

### Glossary

In order to provide a clear and consistent understanding of the terms used in the present disclosure, a number of definitions are provided below. Moreover, unless defined otherwise, all technical and scientific terms as used herein have the same meaning as commonly understood by one of ordinary skill in the art to which this specification pertains.

The word “a” or “an” when used in conjunction with the term “comprising” in the claims and/or the specification may mean “one”, but it is also consistent with the meaning of “one or more”, “at least one”, and “one or more than one” unless the content clearly dictates otherwise. Similarly, the word “another” may mean at least a second or more unless the content clearly dictates otherwise.

As used in this specification and claim(s), the words “comprising” (and any form of comprising, such as “comprise” and “comprises”), “having” (and any form of having, such as “have” and “has”), “including” (and any form of including, such as “include” and “includes”) or “containing” (and any form of containing, such as “contain” and “contains”), are inclusive or open-ended and do not exclude additional, unrecited elements or process steps.

As used in this specification and claim(s), the word “consisting” and its derivatives, are intended to be close ended terms that specify the presence of stated features, elements, components, groups, integers, and/or steps, and also exclude the presence of other unstated features, elements, components, groups, integers and/or steps.

The term “consisting essentially of”, as used herein, is intended to specify the presence of the stated features, elements, components, groups, integers, and/or steps as well as those that do not materially affect the basic and novel characteristic(s) of these features, elements, components, groups, integers, and/or steps.

The terms “about”, “substantially” and “approximately” as used herein mean a reasonable amount of deviation of the modified term such that the end result is not significantly changed. These terms of degree should be construed as including a deviation of at least 1% of the modified term if this deviation would not negate the meaning of the word it modifies.

The term “suitable” as used herein means that the selection of the particular compound or conditions would depend on the specific synthetic manipulation to be performed, and the identity of the molecule(s) to be transformed, but the selection would be well within the skill of a person trained in the art. All process/method steps described herein are to be conducted under conditions sufficient to provide the product shown. A person skilled in the art would understand that all reaction conditions, including, for example, reaction solvent, reaction time, reaction temperature, reaction pressure, reactant ratio and whether or not the reaction should be performed under an anhydrous or inert atmosphere, can be varied to optimize the yield of the desired product and it is within their skill to do so.

The expression “proceed to a sufficient extent” as used herein with reference to the reactions or process steps disclosed herein means that the reactions or process steps proceed to an extent that conversion of the starting material or substrate to product is maximized. Conversion may be maximized when greater than about 5, 10, 15, 20, 25, 30, 35, 40, 45, 50, 55, 60, 65, 70, 75, 80, 85, 90, 95 or 99% of the starting material or substrate is converted to product.

The term “substituted” as used herein, means that a hydrogen radical of the designated moiety is replaced with the radical of a specified substituent, provided that the substitution results in a stable or chemically feasible compound. Non-limiting examples of substituents include halogen (F, Cl, Br, or I) for example F, hydroxyl, thiol, alkylthiol, alkoxy, amino, amido, carboxyl, alkyl, cycloalkyl, arene, heteroarene and cyano.

As used herein, the term “alkyl” can be straight-chain or branched. This also applies if they carry substituents or occur as substituents on other residues, for example in alkoxy residues, alkoxycarbonyl residues or arylalkyl residues. Substituted alkyl residues can be substituted in any suitable position. Examples of alkyl residues containing from 1 to 15 carbon atoms are methyl, ethyl, propyl, butyl, pentyl, hexyl, heptyl, octyl, nonyl, decyl, undecyl, dodecyl, tridecyl, tetradecyl and pentadecyl, the n-isomers of all these residues, isopropyl, isobutyl, isopentyl, neopentyl, isohexyl, isodecyl, 3-methylpentyl, 2,3,4-trimethylhexyl, sec-butyl, tert-butyl, or tert-pentyl. A specific group of alkyl residues is formed by the residues methyl, ethyl, n-propyl, isopropyl, n-butyl, isobutyl, sec-butyl and tert-butyl.

As used herein, the term “lower alkyl” can be straight-chain or branched. This also applies if they carry substituents or occur as substituents on other residues, for example in alkoxy residues, alkoxycarbonyl residues or arylalkyl residues. Substituted alkyl residues can be substituted in any suitable position. Examples of lower alkyl residues containing from 1 to 6 carbon atoms are methyl, ethyl, propyl, isopropyl, butyl, isobutyl, tert-butyl, pentyl, isopentyl, neopentyl, and hexyl.

As used herein, the term “cycloalkyl” is understood as being a carbon-based ring system, non-limiting examples of which include cyclopropyl, cyclobutyl, cyclopentyl and cyclohexyl.

As used herein, the term “cycloalkenyl” is understood as being a carbon-based ring system containing a carbon-carbon double bond, non-limiting examples of which include, cyclobutenyl, cyclopentenyl and cyclohexenyl.

As used herein, the term “alkcycloalkyl” is understood as being a cycloalkyl group attached to the parent molecular group through an alkylene group.

The terms “alkoxy” or “alkyloxy,” as used interchangeably herein, represent an alkyl group attached to the parent molecular group through an oxygen atom.

The term “alkylsulfinyl” as used herein, represents an alkyl group attached to the parent molecular group through an S(O) group.

The term “alkylsulfonyl,” as used herein, represents an alkyl group attached to the parent molecular group through a S(O)₂ group.

The term “alkylthio” as used herein, represents an alkyl group attached to the parent molecular group through a sulfur atom.

The term “alkenyl,” as used herein, represents monovalent straight or branched chain groups of, unless otherwise specified, from 2 to 15 carbons, such as, for example, 2 to 6 carbon atoms or 2 to 4 carbon atoms, containing one or more carbon-carbon double bonds and is exemplified by ethenyl, 1-propenyl, 2-propenyl, 2-methyl-1-propenyl, 1-butenyl, 2-butenyl and the like and may be optionally substituted with one, or more substituents.

The term “alkynyl” as used herein, represents monovalent straight or branched chain groups of from 2 to 15 carbon atoms comprising a carbon-carbon triple bond and is exemplified by ethynyl, 1-propynyl, and the like and may be optionally substituted with one or more substituents.

The term “carbonyl” as used herein, represents a C(O) group, which can also be represented as C═O.

The terms “carboxy” or “carboxyl,” as used interchangeably herein, represents a CO₂H group.

As used herein, the term “arene” is understood as being an aromatic substituent which is a single ring or multiple rings fused together and which is optionally substituted. When formed of multiple rings, at least one of the constituent rings is aromatic. In an embodiment, arene substituents include phenyl, naphthyl, indane, and fluorene groups.

The term “heteroarene” as used herein embraces fully unsaturated or aromatic heterocyclo groups. The heteroarene groups are either monocyclic, bicyclic, tricyclic or quadracyclic, provided they have a suitable number of atoms, for example from 3 to 30 atoms, and are stable. A bicyclic, tricyclic or quadracyclic heteroaryl group is fused, bridged and/or simply linked via a single bond. Examples of heteroarene groups include unsaturated 3 to 6 membered heteromonocyclic groups containing 1 to 4 nitrogen atoms, for example, pyrrolyl, pyrrolinyl, imidazolyl, pyrazolyl, pyridyl, pyrimidyl, pyrazinyl, pyridazinyl, triazolyl (e.g., 4H-1,2,4-triazolyl, 1H-1,2,3-triazolyl, 2H-1,2,3-triazolyl, etc.), tetrazolyl (e.g. 1H-tetrazolyl, 2H-tetrazolyl, etc.), etc.; unsaturated condensed heterocyclo groups containing 1 to 5 nitrogen, oxygen and/or sulfur atoms including, for example, indolyl, isoindolyl, indolizinyl, benzimidazolyl, quinolyl, isoquinolyl, indazolyl, benzotriazolyl, tetrazolopyridazinyl (e.g., tetrazolo[1,5-b]pyridazinyl, etc.), etc.; unsaturated 3 to 6-membered heteromonocyclic groups containing an oxygen atom, including, for example, pyranyl, furyl, etc.; unsaturated 3 to 6-membered heteromonocyclic groups containing a sulfur or a selenium atom, including for example, thienyl, selenophen-yl, etc.; unsaturated 3- to 6-membered heteromonocyclic groups containing 1 to 2 oxygen atoms and 1 to 3 nitrogen atoms, including, for example, oxazolyl, isoxazolyl, oxadiazolyl (e.g., 1,2,4-oxadiazolyl, 1,3,4-oxadiazolyl, 1,2,5-oxadiazolyl, etc.) etc.; unsaturated condensed heterocyclo groups containing 1 to 2 oxygen atoms and 1 to 3 nitrogen atoms (e.g. benzoxazolyl, benzoxadiazolyl, etc.); unsaturated 3 to 6-membered heteromonocyclic: groups containing 1 to 2 sulfur atoms and 1 to 3 nitrogen atoms, including, for example, thiazolyl, thiadiazolyl (e.g., 1,2,4-thiadiazolyl, 1,3,4-thiadiazolyl, 1,2,5-thiadiazolyl, etc.) etc.; unsaturated condensed heterocyclo groups containing 1 to 2 sulfur atoms and 1 to 3 nitrogen atoms (e.g., benzothiazolyl, benzothiadiazolyl, etc.), unsaturated linked 5 or 6-membered heteromonocyclic groups containing 1 to 2 sulfur atoms and/or 1 to 3 nitrogen atoms, including, for example, bithienyl and trithienyl and the like. The term also embraces groups where heterocyclo groups are fused with aryl groups. Examples of such fused bicyclic groups include benzofuran, benzothiophene, benzopyran, and the like.

The term “pharmaceutically acceptable salt,” as used herein, refers to those salts which are, within the scope of sound medical judgment, suitable for use in contact with the tissues of humans without undue toxicity, irritation, allergic response and the like, and are commensurate with a reasonable benefit/risk ratio. Pharmaceutically acceptable salts are well-known in the art. The salts can be prepared in situ during the final isolation of the compounds, or separately by reacting the free base or acid function with a suitable organic acid or base, respectively. Representative acid addition salts include acetate, adipate, alginate, ascorbate, aspartate, benzenesulfonate, benzoate, bisulfate, borate, butyrate, camphorate, camphersulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts, and the like. Basic addition salts can be prepared during the final isolation and purification of the compounds by reacting a carboxy group (or other acidic moiety) with a suitable base such as the hydroxide, carbonate, or bicarbonate of a metal cation or with ammonia or an organic primary, secondary, or tertiary amine. The cations of pharmaceutically acceptable salts include lithium, sodium, potassium, calcium, magnesium, and aluminum, as well as nontoxic quaternary amine cations such as ammonium, tetramethylammonium, tetraethylammonium, methylamine, dimethylamine, trimethylamine, triethylamine, diethylamine, ethylamine, tributylamine, pyridine, N,N-dimethylaniline, N-methylpiperidine, N-methylmorpholine, dicyclohexylamine, procaine, dibenzylamine, N,N-dibenzylphenethylamine, 1-ephenamine, and N,N′-dibenzylethylenediamine. Other representative organic amines useful for the formation of base addition salts include ethylenediamine, ethanolamine, diethanolamine, piperidine and piperazine.

The term “derivative” as used herein, is understood as being a substance which comprises the same basic carbon skeleton and carbon functionality in its structure as a given compound, but can also bear one or more substituents or rings.

The term “analogue” as used herein, is understood as being a substance similar in structure to another compound but differing in some slight structural detail.

As used herein, the term “bioisostere” shall refer to a compound resulting from the exchange of an atom or of a group of atoms with another, broadly similar, atom or group of atoms. Such an exchange is termed a “bioisosteric replacement” and is useful to create a new compound with similar biological properties to the parent compound. Bioisosteric replacement generally enhances desired biological or physical properties of a compound without making significant changes in chemical structure. For example, the replacement of a hydrogen atom with a fluorine atom at a site of metabolic oxidation in a drug candidate may prevent such metabolism from taking place. Because the fluorine atom is similar in size to the hydrogen atom the overall topology of the molecule is not significantly affected, leaving the desired biological activity unaffected. However, with a blocked pathway for metabolism, the drug candidate may have a longer half-life. Another example is aromatic rings, a phenyl —C₆H₅ ring can often be replaced by a different aromatic ring such as thiophene or naphthalene which may improve efficacy or change binding specificity of a respective bioisostere.

In an aspect, the present disclosure broadly relates to novel urea compounds and bioisosteres thereof and to their use in treating, attenuating, inhibiting and/or preventing inflammation and inflammation-related pathologies. In an embodiment, the present disclosure relates to novel urea compounds and bioisosteres thereof and to their use in treating, attenuating, inhibiting or preventing conditions associated with the expression of IL-6. In an embodiment, the present disclosure relates to compounds of Formulas I-VI and to their use in treating, attenuating, inhibiting and/or preventing inflammation and inflammation-related pathologies. In an embodiment, the present disclosure relates to compounds of Formulas I-VI and to their use in treating, attenuating, inhibiting and/or preventing conditions associated with the expression of IL-6. In a further embodiment, the present disclosure relates to pharmaceutical compositions comprising one or more compounds of Formulas I-VI and to their use in treating, attenuating, inhibiting and/or preventing inflammation and inflammation-related pathologies. In a further embodiment, the present disclosure relates to pharmaceutical compositions comprising one or more compounds of Formulas I-VI and to their use in treating, attenuating, inhibiting or preventing conditions associated with the expression of IL-6. In a further embodiment, the present disclosure relates to pharmaceutical compositions comprising one or more substituted phenyl cycloalkylureas and to their use in treating, attenuating, inhibiting and/or preventing inflammation and inflammation-related pathologies. In a further embodiment, the present disclosure relates to pharmaceutical compositions comprising one or more substituted phenyl cycloalkylureas and to their use in treating, attenuating, inhibiting or preventing conditions associated with the expression of IL-6. In a further embodiment, the present disclosure relates to intermediates and processes for the synthesis of compounds of Formulas I-VI.

In an embodiment of the present disclosure, the compounds of Formulas I-VI can be used to treat, attenuate, inhibit and/or prevent conditions associated with the expression of IL-6 in a patient in need of such therapy. The compounds of Formulas I-VI can be used alone or they can be used as part of a multi-drug regimen in combination with known therapeutics.

In an embodiment of the present disclosure, the compounds of Formulas I-VI comprise pharmaceutically acceptable solvates thereof. Many of the compounds of Formulas I-VI can combine with solvents such as water, methanol, ethanol and acetonitrile to form pharmaceutically acceptable solvates such as the corresponding hydrate, methanolate, ethanolate and acetonitrilate.

In an aspect, the present disclosure relates to pharmaceutical compositions comprising one or more compounds of Formulas I-VI and a pharmaceutically acceptable carrier, diluent, or excipient. The pharmaceutical compositions are prepared by known procedures using well-known and readily available ingredients.

In an embodiment of the present disclosure, the compounds of Formulas I-VI or pharmaceutical compositions comprising the compounds of Formulas I-VI may be administered topically or percutaneously in dosage unit formulations containing conventional non-toxic pharmaceutically acceptable carriers, adjuvants and vehicles. In the usual course of therapy, the compounds of Formulas I-VI are incorporated into an acceptable vehicle to form a composition for administration to the affected area, such as hydrophobic or hydrophilic creams or lotions, or into a form suitable for percutaneous administration.

In general, the route of administration of the compounds of Formulas I-VI is topical (including administration to the skin and scalp), or percutaneously. Topical administration is usually the most effective for the treatment of psoriasis where such direct application is practical. Shampoo formulations can be advantageous for treating psoriasis of the scalp. The present disclosure contemplates the administration of one or more compounds of Formulas I-VI either alone or in combination with other therapeutics.

The dosage to be administered is not subject to defined limits, but it will usually be an effective amount. It will usually be an amount sufficient to achieve a desired pharmacological and physiological effect. The pharmaceutical compositions of the present disclosure comprise a pharmaceutically effective amount of at least one compound of Formulas I-VI or pharmaceutically acceptable salt thereof as described herein and one or more pharmaceutically acceptable carriers, excipients or diluents. In an embodiment of the present disclosure, the pharmaceutical compositions contain from about 0.1% to about 99% by weight of a compound of Formulas I-VI or pharmaceutically acceptable salt thereof as disclosed herein. In a further embodiment of the present disclosure, the pharmaceutical compositions contain from about 10% to about 60% by weight of a compound of Formulas I-VI or pharmaceutically acceptable salt thereof as disclosed herein. Physicians will determine the most-suitable dosage of the compounds of Formulas I-VI or pharmaceutically acceptable salts thereof.

Previous work by Applicant on the ASK-p38 downstream pathway suggested that CEUs could possibly impact the expression of IL-6. Knowing that minor structural modifications/substitutions, notably on the phenyl ring and/or on the R₁ part of the compounds of the present disclosure, may greatly impact the biological properties of the compounds, initial experiments were conducted using tBCEU and cHCEU. In an embodiment of the present disclosure, the effect of tBCEU and cHCEU on the expression of IL-6 by HaCaT cells, a human aneuploid immortal keratinocyte cell line treated with IL-17 and TNFα, confirmed a significant decrease on the expression of IL-6. In order to further assess the activity of this class of compounds, a series of novel substituted phenyl cycloalkylureas (4a-6e), in accordance with various embodiments of the present disclosure, were prepared. A general procedure for the synthesis of various substituted phenyl cycloalkylureas in accordance with an embodiment of the present disclosure, is illustrated in Scheme 1. Compounds 4a-6e were prepared in low to good yields (10-66%) by nucleophilic addition of anilines 1a-1e to the cycloalkyl isocyanates 2a-2d.

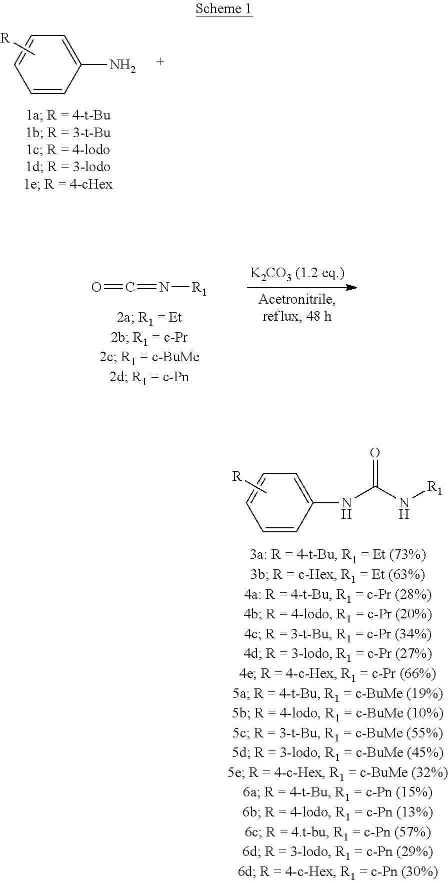

The effect of compounds 3a-b, 4a-e, 5a-e and 6a-e on the expression of IL-6 was subsequently assessed using the HaCaT spontaneously transformed aneuploid immortal keratinocyte cell line, stimulated by the addition of IL-17A and TNFα in the culture medium (Table 1). Curcumin and ibuprofen were used as known IL-6 expression inhibitors. DMSO was used for solubilizing the drugs. The in vitro inhibitory data revealed that substituted phenyl cycloalkylureas 3b, 4b-e, 5b, 5c, 5e and 6e at 10 μM had a significant inhibitory effect on IL-6 expression in HaCaT cells stimulated with IL-17A and TNFα. Indeed, the inhibitory effect of these molecules was at least equivalent to that of curcumin [27] and ibuprofen, both recognized as anti-inflammatory drugs [28]. The inhibitory effect of the substituted phenyl cycloalkylureas in accordance with an embodiment of the present disclosure on proinflammatory cytokines was also found at the mRNA level. Indeed, the mRNAs of the cytokines IL-6 and TNF-α as well as the mRNA of the chemokine IL-8 were lowered in TNF-α/IL-17A-stimulated HaCaT cells. Similar results were obtained in THP-1 cells stimulated with LPS where treatments with CHEU and 4e diminished the mRNA levels of the cytokines IL-1B, IL-6 and TNF-α in addition to the mRNA levels of the chemokine IL-8. It is surmised that the anti-inflammatory mechanism of action of the compounds of the present disclosure is likely mediated, at least in part, by modulating the phosphorylation state of the MAP kinase p38. The level of phospho-p38 (p-p38) is rapidly increased (approx. 15 min.) following stimulation of HaCaT cells with the cytokines TNF-α and IL-17A. Addition of a compound in accordance with an embodiment of the present disclosure, prior to stimulation with the aforementioned cytokines, decreases the level of p-p38 at 30-60 minutes post-stimulation. This diminution in p-p38 levels destabilizes the mRNAs of pro-inflammatory cytokines, non-limiting examples of which include TNF-α and IL-6. Moreover, it is surmised that the effect of the compounds of the present disclosure on p-p38 is potentially mediated by the activation of the phosphatase named DUSP1.

TABLE 1
Effect on IL-6 expression for 10 μM solutions of tBCEU, cHCEU,
compounds 3a-b, 4a-e, 5a-e, 6a-e, curcumin and ibuprofen.

Expression of IL-6^{a} (pg/mL)

| Compound | HaCaT | Adjust Pvalue |
|---|---|---|
| tBCEU | 130.6 ± 15.6 | >0.9999 |
| 3a | 81.9 ± 10.8 | 0.5465 |
| cHCEU | 46.8 ± 8.5 | 0.0001 |
| 3b | 36.2 ± 7.4 | <0.0001 |
| 4a | 91.9 ± 7.6 | >0.9999 |
| 4b | 47.9 ± 4.5 | 0.0003 |
| 4c | 50.4 ± 2.6 | 0.0009 |
| 4d | 57.3 ± 4.4 | 0.0086 |
| 4e | 29.2 ± 3.5 | <0.0001 |
| 5a | 70.6 ± 7.6 | 0.1507 |
| 5b | 33.9 ± 4.3 | 0.0001 |
| 5c | 47.5 ± 3.3 | 0.0003 |
| 5d | 90.2 ± 11.1 | >0.9999 |
| 5e | 64.1 ± 5.4 | 0.0528 |
| 6a | 127.5 ± 17.3 | >0.9999 |
| 6b | 65.1 ± 7.6 | 0.0382 |
| 6c | 74.6 ± 6.7 | 0.3709 |
| 6d | 89.8 ± 9.0 | >0.9999 |
| 6e | 50.3 ± 4.7 | 0.0024 |
| IL-17α/ | 177.9 ± 20.3 | |
| TNFα | | |
| DMSO | 29.2 ± 7.1 | <0.0001 |
| Curcumin | 62.8 ± 12.0 | 0.0064 |
| Ibuprofen | 120.3 ± 18.6 | >0.9999 |

^{a}Values are means of five separate experiments conducted in duplicate

The antiproliferative activity of compounds 3a-b, 4a-e, 5a-e and 6a-e was subsequently evaluated using the human HT-29 colon adenocarcinoma and the A549 adenocarcinoma alveolar epithelial cancer cell lines, the HaCaT spontaneously transformed aneuploid immortal keratinocyte cell line and the HDFn neonatal dermal fibroblast cell line (Table 2). Cell growth inhibition was assessed according to the NCI/NIH Developmental Therapeutics Program [29]. Furthermore, compounds 3a-b, 4a-e, 5a-e and 6a-e showed low to very low antiproliferative activity on the cancer and primary cell lines tested. Interestingly, these molecules were shown to exhibit lower antiproliferative activity than curcumin.

TABLE 2
Antiproliferative activity of tBCEU, cHCEU and compounds
3a-6e on the HT-29, HaCaT, HDFn and A549 cell lines.

Antiproliferative activity (GI₅₀, μM)^{a}

| Compound | HT-29 | HaCaT | HDFn | A549 |
|---|---|---|---|---|
| tBCEU | 6.5 | 12 | 11 | 6.9 |
| 3a | 75 | >100 | 60 | 69 |
| cHCEU | 15 | 27 | 11 | 12 |
| 3b | 23 | >100 | 92 | 54 |
| 4a | 85 | >100 | 55 | >100 |
| 4b | 82 | >100 | 95 | >100 |
| 4c | >100 | >100 | >100 | >100 |
| 4d | >100 | >100 | 79 | 84 |
| 4e | 54 | 74 | 40 | 48 |
| 5a | 21 | 35 | 8.2 | >100 |
| 5b | 29 | 62 | 74 | 27 |
| 5c | 43 | 39 | 21 | 37 |
| 5d | 25 | 31 | 15 | 25 |
| 5e | >100 | >100 | >100 | >100 |
| 6a | >100 | >100 | >100 | 83 |
| 6b | 9.7 | >100 | >100 | 62 |
| 6c | 27 | >100 | 84 | 34 |
| 6d | 30 | 49 | 23 | 30 |
| 6e | 13 | 82 | 56 | 11 |
| Curcumin | 4.2 | 7.0 | 7.6 | 9.9 |
| Ibuprofen | >100 | >100 | >100 | >100 |

^{a}Values are means of two separate experiments conducted in triplicate and the deviation from the mean is <10%

The effect of compounds 3a-b, 4e, 5b-c, 6a and 6e on cell cycle progression was subsequently assessed using a flow cytometer in accordance with established experimental protocols [30-32] to evaluate any potential drug toxicity at specific phases of the cell cycle (Table 3). Compounds 3a-b, 4e, 5b-c, 6a and 6e did not affect the cell cycle progression whereas curcumin is clearly arresting the cell cycle progression in the G2-M phase similarly to tBCEU which are both known to act as an antimicrotubule agents.

TABLE 3
Effect of compounds 3a-b, 4e, 5b-c,
6a and 6e on cell cycle progression.

| | Cell cycle progression^{a} | | |
|---|---|---|---|
| Compound | G₀-G₁ | S | G₂-M |
| 3a | 44 | 25.1 | 31 |
| 3b | 46.9 | 25.3 | 27.8 |
| 4e | 47.7 | 24.2 | 28.1 |
| 5b | 45 | 25.6 | 29.4 |
| 5c | 46.1 | 23.5 | 30.4 |
| 6a | 48 | 25.1 | 25.8 |
| 6e | 50.1 | 24.7 | 25.2 |
| DMSO | 45.7 | 24.8 | 29.5 |
| Curcumin | 14.8 | 8.1 | 77.1 |
| Ibuprofen | 57.9 | 25.6 | 16.3 |
| tBCEU | 57.3 | 26.8 | 15.6 |
| tBEU | 55.5 | 25.3 | 19.1 |
| cHCEU | 56.5 | 24.4 | 19.1 |

^{a}Values are the means of two separate experiments conducted in triplicate and the deviation from the mean value is <10% (Cell cycle analysis performed using HaCaT cells)

A general procedure for the synthesis of various substituted phenyl alkylthioureas in accordance with an embodiment of the present disclosure, is illustrated in Scheme 2. The compounds were prepared in low to good yields by nucleophilic addition of anilines to alkylisothiocyanates.

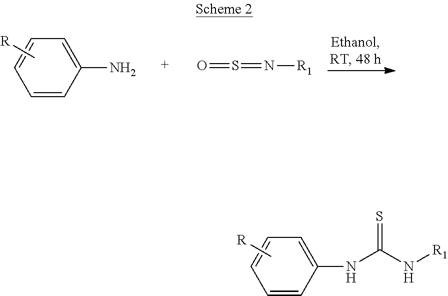

Desired substituted anilines (50 mg, 1.0 eq.) were dissolved in acetonitrile (6 ml) followed by the addition of K₂CO₃ (1.2 eq.) and an isothiocyanate (1.2 eq.). The resulting reaction mixture was stirred for 48 h under reflux. The reaction mixture was subsequently evaporated to dryness under reduced pressure and the residue purified by flash chromatography on silica gel. Alternatively, desired substituted anilines (50 mg, 1.0 eq.) were dissolved in ethanol (2 ml) followed by the addition of an isothiocyanate (1.2 eq.). The resulting reaction mixture was stirred for 48 h at room temperature. The reaction mixture was subsequently evaporated to dryness under reduced pressure and the residue purified by flash chromatography on silica gel.

A general procedure for the synthesis of various substituted phenyl alkyl squaramides in accordance with an embodiment of the present disclosure, is illustrated in Scheme 3. The compounds were prepared in low to good yields by the addition of anilines to dialkoxysquarate.

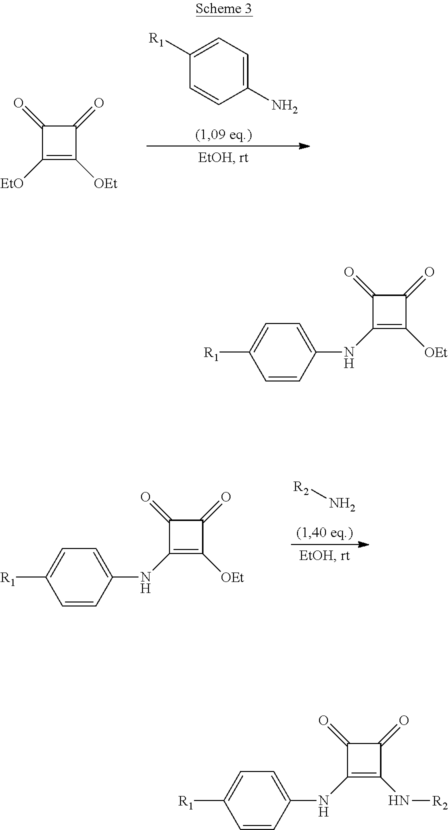

To a solution of the desired aniline (200 mg, 1.0 eq.) in EtOH (2.5 mL) at rt was added dropwise diethoxysquarate (1.09 eq.). The solution was first stirred at 0° C. for 2 hours, then at rt for 48 h and finally cooled down again to 0° C. The reaction mixture was evaporated to dryness under reduced pressure and the residue purified by flash chromatography on silica gel using hexanes/ethyl acetate (80/20) yielding the desired amino alkoxysquarate. The amino alkoxysquarate (40 mg, 1 eq.) was subsequently dissolved in EtOH (2 mL) at rt followed by the addition of an amine (1.4 eq.). The resulting mixture was stirred for 48 h at room temperature and then filtered. The solid residue was washed with a mixture of cold EtOH/MeOH (1/1) to afford the desired squaramide without further purification.

A representative number of substituted phenyl alkylureas, substituted phenyl alkylthioureas and substituted phenyl alkyl squaramides in accordance with various embodiments of the present disclosure are illustrated in Table 4.

TABLE 4
Selected substituted phenyl alkylureas, substituted phenyl alkylthioureas and
substituted phenyl alkyl squaramides

| | | | Yield | Melting Point | Exact |
|---|---|---|---|---|---|
| Compound | Structure | Appearance | (%) | (° C.) | Mass |
| 1 | 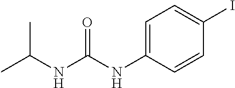 | White solid | 100 | Decomposition 237-249 | 304.01 |
| 2 | 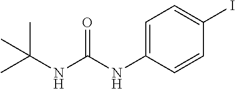 | White Solid | 44 | 179-199 | 318.02 |
| 3 | 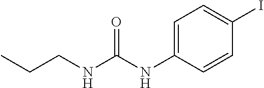 | White Solid | 61 | 197-199 | 304.01 |
| 4 | 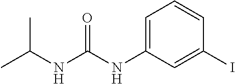 | White Solid | 97 | 153-156 | 304.01 |
| 5 | 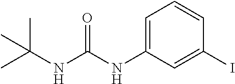 | White Solid | 83 | 189-193 | 318.02 |
| 6 | 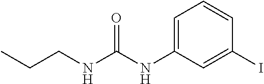 | White Solid | 100 | 132-136 | 304.01 |
| 7 | 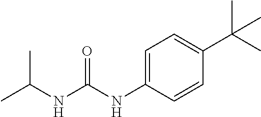 [SMILES: CC(C)NC(=O)Nc1ccc(C(C)(C)C)cc1] | White Solid | 90 | 152-155 | 234.17 |
| 8 | 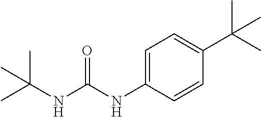 [SMILES: CC(C)(C)NC(=O)Nc1ccc(C(C)(C)C)cc1] | White Solid | 78 | 167-174 | 248.19 |
| 9 | 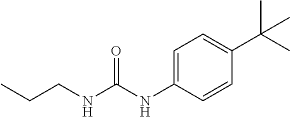 [SMILES: CCCNC(=O)Nc1ccc(C(C)(C)C)cc1] | White Solid | 100 | 147-151 | 234.17 |
| 10 | 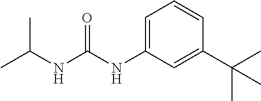 [SMILES: CC(C)NC(=O)Nc1cccc(C(C)(C)C)c1] | White Solid | 81 | 146-149 | 234.17 |
| 11 | 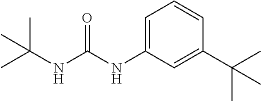 [SMILES: CC(C)(C)NC(=O)Nc1cccc(C(C)(C)C)c1] | Light brown solid | 70 | 161-170 | 248.19 |
| 12 | 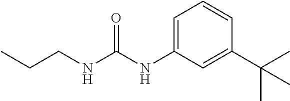 [SMILES: CCCNC(=O)Nc1cccc(C(C)(C)C)c1] | Light brown solid | 88 | 138-141 | 235.17 |
| 13 | 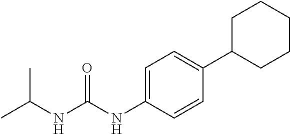 [SMILES: CC(C)NC(=O)Nc1ccc(C2CCCCC2)cc1] | White Solid | 80 | 163-170 | 260.19 |
| 14 | 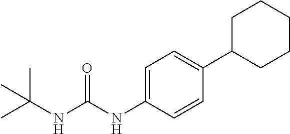 [SMILES: CC(C)(C)NC(=O)Nc1ccc(C2CCCCC2)cc1] | White Solid | 77 | 182-189 | 274.20 |
| 15 | 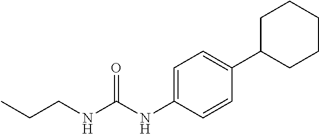 [SMILES: CCCNC(=O)Nc1ccc(C2CCCCC2)cc1] | White Solid | 100 | 166-168 | 260.19 |
| 16 | 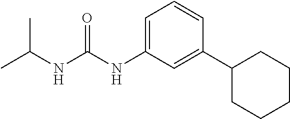 [SMILES: CC(C)NC(=O)Nc1cccc(C2CCCCC2)c1] | White Solid | 86 | 178-181 | 260.19 |
| 17 | 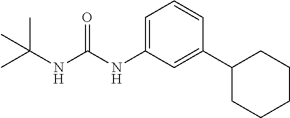 [SMILES: CC(C)(C)NC(=O)Nc1cccc(C2CCCCC2)c1] | White Solid | 54 | 195-203 | 274.20 |
| 18 | 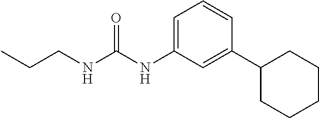 [SMILES: CCCNC(=O)Nc1cccc(C2CCCCC2)c1] | White Solid | 91 | 131-134 | 260.19 |
| 19 | 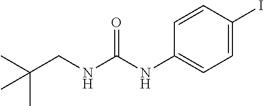 [SMILES: CC(C)(C)CNC(=O)Nc1ccc(I)cc1] | Sticky Solid | 11.3 | | 332.04 |
| 20 | 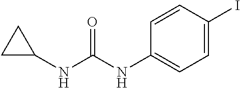 [SMILES: O=C(Nc1ccc(I)cc1)NC1CC1] | White Solid | 19.6 | 216-218 | 301.99 |
| 21 | 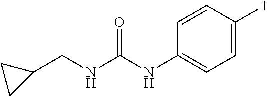 [SMILES: O=C(NCC1CC1)Nc1ccc(I)cc1] | White Solid | 26 | 192-194 | 316.01 |
| 22 | 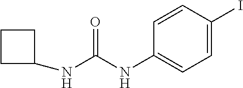 [SMILES: O=C(Nc1ccc(I)cc1)NC1CCC1] | White Solid | 12.5 | 208-210 | 316.01 |
| 23 | 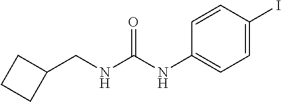 [SMILES: O=C(NCC1CCC1)Nc1ccc(I)cc1] | White Solid | 10 | 159-160 | 330.02 |
| 24 | 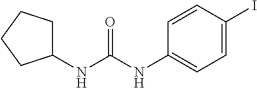 [SMILES: O=C(Nc1ccc(I)cc1)NC1CCCC1] | White Solid | 13.4 | 188-191 | 330.02 |
| 25 | 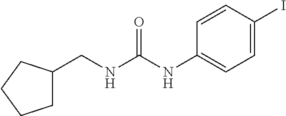 [SMILES: O=C(NCC1CCCC1)Nc1ccc(I)cc1] | White Solid | 9.5 | 158-161 | 344.04 |
| 26 | 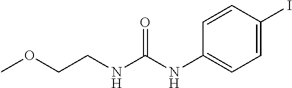 | White Solid | 19.7 | 141-143 | 320.00 |
| 27 | 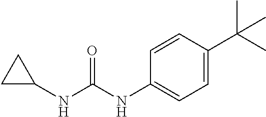 [SMILES: CC(C)(C)c1ccc(NC(=O)NC2CC2)cc1] | Light yellow solid | 27.7 | 138-139 | 232.16 |
| 28 | 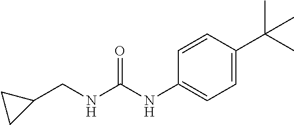 [SMILES: CC(C)(C)c1ccc(NC(=O)NCC2CC2)cc1] | White solid | 16.5 | 168-170 | 246.17 |
| 29 | 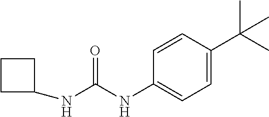 [SMILES: CC(C)(C)c1ccc(NC(=O)NC2CCC2)cc1] | White solid | 28.9 | 189-190 | 246.17 |
| 30 | 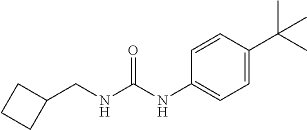 [SMILES: CC(C)(C)c1ccc(NC(=O)NCC2CCC2)cc1] | White solid | 19.1 | 179-184 | 260.19 |
| 31 | 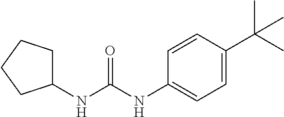 [SMILES: CC(C)(C)c1ccc(NC(=O)NC2CCCC2)cc1] | White solid | 30.0 | 218-223 | 260.19 |
| 32 | 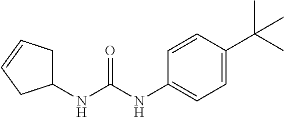 [SMILES: CC(C)(C)c1ccc(NC(=O)NC2CC=CC2)cc1] | White solid | 34.1 | 189-191 | 258.17 |
| 33 | 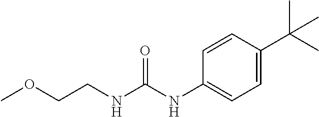 [SMILES: COCCNC(=O)Nc1ccc(C(C)(C)C)cc1] | Light yellow solid | 29.2 | 122-124 | 250.17 |
| 34 | 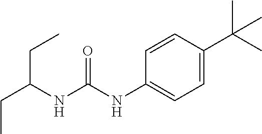 [SMILES: CCC(CC)NC(=O)Nc1ccc(C(C)(C)C)cc1] | White solid | 20.6 | 119-121 | 262.20 |
| 35 | 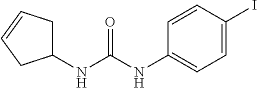 [SMILES: O=C(Nc1ccc(I)cc1)NC1CC=CC1] | White solid | 15 | 201-202 | 328.01 |
| 36 | 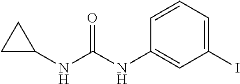 [SMILES: O=C(Nc1cccc(I)c1)NC1CC1] | White solid | 27.2 | 153-154 | 301.99 |
| 37 | 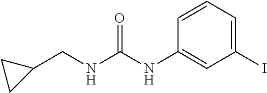 [SMILES: O=C(NCC1CC1)Nc1cccc(I)c1] | White solid | 15.2 | 139-140 | 316.01 |
| 38 | 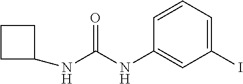 [SMILES: O=C(Nc1cccc(I)c1)NC1CCC1] | White solid | 15.6 | 178-180 | 316.01 |
| 39 | 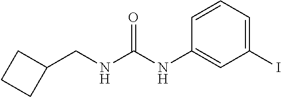 [SMILES: O=C(NCC1CCC1)Nc1cccc(I)c1] | White solid | 45.3 | 138-140 | 330.02 |
| 40 | 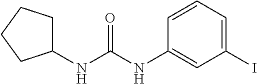 [SMILES: O=C(Nc1cccc(I)c1)NC1CCCC1] | White solid | 15.8 | 142-143 | 330.02 |
| 41 | 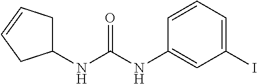 [SMILES: O=C(Nc1cccc(I)c1)NC1CC=CC1] | Light brown solid | 29.2 | 151-153 | 328.01 |
| 42 | 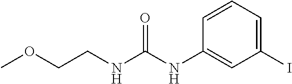 | Light yellow solid | 16.3 | 99-101 | 320.00 |
| 43 | 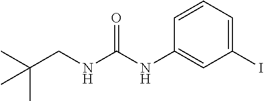 [SMILES: CC(C)(C)CNC(=O)Nc1cccc(I)c1] | White solid | 45.3 | 114-115 | 332.04 |
| 44 | 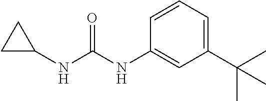 [SMILES: CC(C)(C)c1cccc(NC(=O)NC2CC2)c1] | White solid | 34.2 | 169-171 | 232.16 |
| 45 | 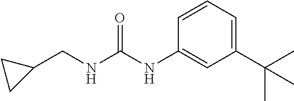 [SMILES: CC(C)(C)c1cccc(NC(=O)NCC2CC2)c1] | White solid | 43.5 | 165-166 | 246.17 |
| 46 | 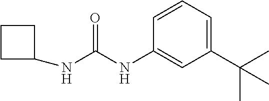 [SMILES: CC(C)(C)c1cccc(NC(=O)NC2CCC2)c1] | White solid | 56.9 | 165-166 | 246.17 |
| 47 | 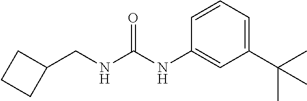 [SMILES: CC(C)(C)c1cccc(NC(=O)NCC2CCC2)c1] | White solid | 54.9 | 126-127 | 260.19 |
| 48 | 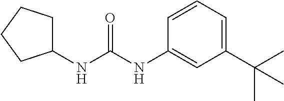 [SMILES: CC(C)(C)c1cccc(NC(=O)NC2CCCC2)c1] | White solid | 57.3 | 152-154 | 260.19 |
| 49 | 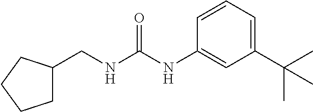 [SMILES: CC(C)(C)c1cccc(NC(=O)NCC2CCCC2)c1] | White solid | 53.1 | 113-114 | 274.20 |
| 50 | 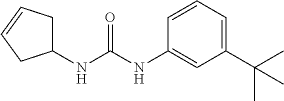 [SMILES: CC(C)(C)c1cccc(NC(=O)NC2CC=CC2)c1] | White solid | 33.6 | 135-137 | 258.17 |
| 51 | 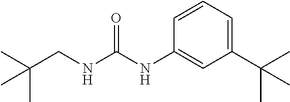 [SMILES: CC(C)(C)CNC(=O)Nc1cccc(C(C)(C)C)c1] | White solid | 26.4 | 145-147 | 262.20 |
| 52 | 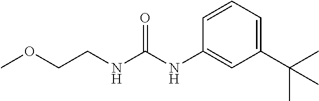 [SMILES: COCCNC(=O)Nc1cccc(C(C)(C)C)c1] | White solid | 60.9 | 119-120 | 250.17 |
| 53 | 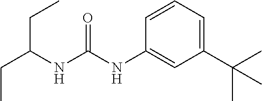 [SMILES: CCC(CC)NC(=O)Nc1cccc(C(C)(C)C)c1] | White solid | 11.4 | 159-161 | 262.20 |
| 54 | 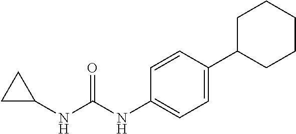 [SMILES: O=C(Nc1ccc(C2CCCCC2)cc1)NC1CC1] | White solid | 66.2 | 173-176 | 258.17 |
| 55 | 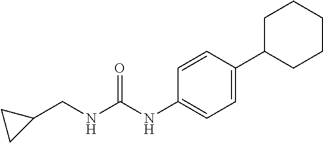 [SMILES: O=C(NCC1CC1)Nc1ccc(C2CCCCC2)cc1] | White solid | 72.0 | 178-181 | 272.19 |
| 56 | 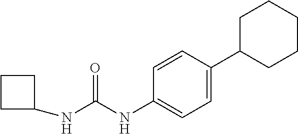 [SMILES: O=C(Nc1ccc(C2CCCCC2)cc1)NC1CCC1] | White solid | 11.0 | 137-139 | 272.19 |
| 57 | 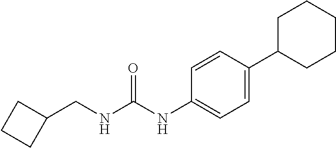 [SMILES: O=C(NCC1CCC1)Nc1ccc(C2CCCCC2)cc1] | White solid | 32.1 | 172-173 | 286.20 |
| 58 | 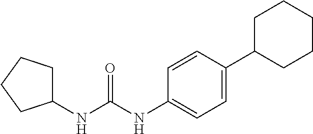 [SMILES: O=C(Nc1ccc(C2CCCCC2)cc1)NC1CCCC1] | White solid | 29.8 | 197-199 | 286.20 |
| 59 | 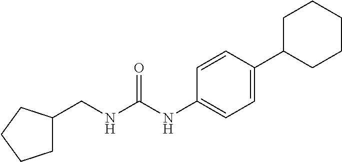 [SMILES: O=C(NCC1CCCC1)Nc1ccc(C2CCCCC2)cc1] | White solid | 13.5 | 142-144 | 300.22 |
| 60 | 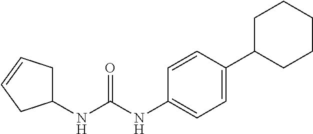 [SMILES: O=C(Nc1ccc(C2CCCCC2)cc1)NC1CC=CC1] | White solid | 41.1 | 167-169 | 284.19 |
| 61 | 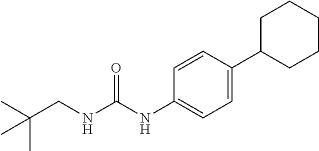 [SMILES: CC(C)(C)CNC(=O)Nc1ccc(C2CCCCC2)cc1] | White solid | 20.8 | 167-169 | 282.22 |
| 62 | 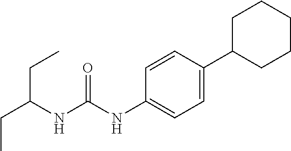 [SMILES: CCC(CC)NC(=O)Nc1ccc(C2CCCCC2)cc1] | White solid | 13.5 | 174-176 | 282.22 |
| 63 | 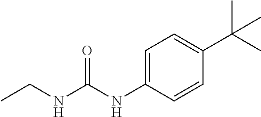 [SMILES: CCNC(=O)Nc1ccc(C(C)(C)C)cc1] | White solid | 73.0 | 136-139 | 220.16 |
| 64 | 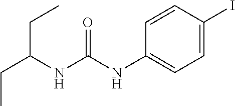 [SMILES: CCC(CC)NC(=O)Nc1ccc(I)cc1] | White solid | 8.0 | 174-176 | 332.04 |
| 65 | 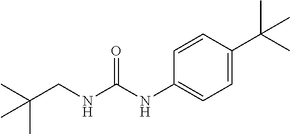 [SMILES: CC(C)(C)CNC(=O)Nc1ccc(C(C)(C)C)cc1] | White solid | 51.9 | 185-187 | 262.20 |
| 66 | 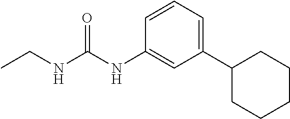 [SMILES: CCNC(=O)Nc1cccc(C2CCCCC2)c1] | White solid | 64.0 | 141-144 | 246.17 |
| 67 | 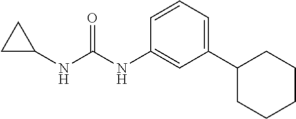 [SMILES: O=C(Nc1cccc(C2CCCCC2)c1)NC1CC1] | White solid | 100.0 | 138-144 | 258.17 |
| 68 | 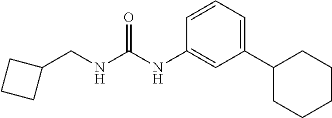 [SMILES: O=C(NCC1CCC1)Nc1cccc(C2CCCCC2)c1] | White solid | 89.0 | 115-120 | 286.20 |
| 69 | 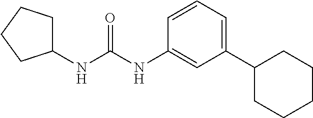 [SMILES: O=C(Nc1cccc(C2CCCCC2)c1)NC1CCCC1] | White solid | 54.0 | 132-137 | 286.20 |
| 70 | 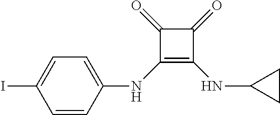 [SMILES: O=c1c(Nc2ccc(I)cc2)c(NC2CC2)c1=O] | Light yellow solid | 75.3 | Decomposition 269-278 | 353.99 |
| 71 | 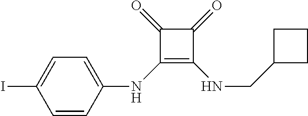 [SMILES: O=c1c(NCC2CCC2)c(Nc2ccc(I)cc2)c1=O] | White solid | 89.8 | Decomposition 274-294 | 382.02 |
| 72 | 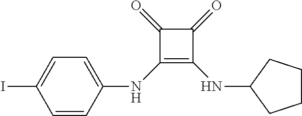 [SMILES: O=c1c(Nc2ccc(I)cc2)c(NC2CCCC2)c1=O] | Light yellow solid | 90.4 | Decomposition 260-268 | 382.02 |
| 73 | 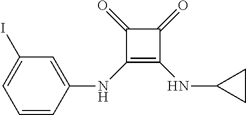 [SMILES: O=c1c(Nc2cccc(I)c2)c(NC2CC2)c1=O] | Light brown solid | 52.6 | Decomposition 233-276 | 353.99 |
| 74 | 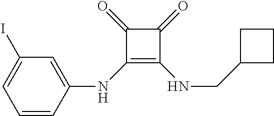 [SMILES: O=c1c(NCC2CCC2)c(Nc2cccc(I)c2)c1=O] | Light yellow solid | 55.5 | Decomposition 231-277 | 382.02 |
| 75 | 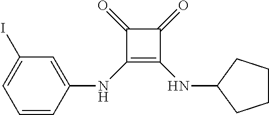 [SMILES: O=c1c(Nc2cccc(I)c2)c(NC2CCCC2)c1=O] | Light yellow solid | 80.1 | Decomposition 230-274 | 382.02 |
| 76 | 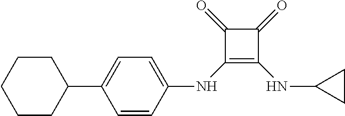 [SMILES: O=c1c(Nc2ccc(C3CCCCC3)cc2)c(NC2CC2)c1=O] | Light brown solid | 99.0 | Decomposition 232-247 | 310.17 |
| 77 | 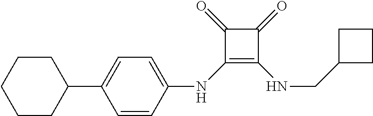 [SMILES: O=c1c(NCC2CCC2)c(Nc2ccc(C3CCCCC3)cc2)c1=O] | White solid | 82.3 | Decomposition 285-316 | 338.20 |
| 78 | 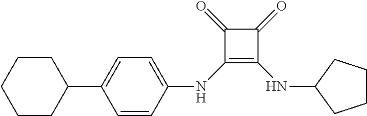 [SMILES: O=c1c(Nc2ccc(C3CCCCC3)cc2)c(NC2CCCC2)c1=O] | White solid | 85.4 | Decomposition 268-329 | 338.20 |
| 79 | 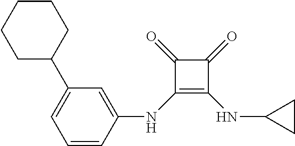 [SMILES: O=c1c(Nc2cccc(C3CCCCC3)c2)c(NC2CC2)c1=O] | Light brown solid | | | 310.17 |
| 80 | 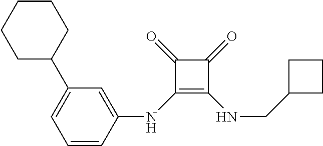 [SMILES: O=c1c(NCC2CCC2)c(Nc2cccc(C3CCCCC3)c2)c1=O] | White solid | | | 338.20 |
| 81 | 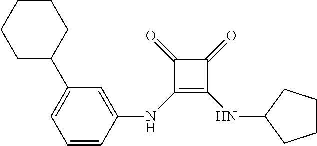 [SMILES: O=c1c(Nc2cccc(C3CCCCC3)c2)c(NC2CCCC2)c1=O] | White solid | | | 338.20 |
| 82 | 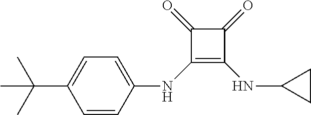 [SMILES: CC(C)(C)c1ccc(Nc2c(NC3CC3)c(=O)c2=O)cc1] | White solid | 75.0 | Decomposition 186-258 | 284.15 |
| 83 | 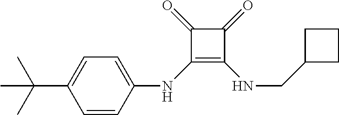 [SMILES: CC(C)(C)c1ccc(Nc2c(NCC3CCC3)c(=O)c2=O)cc1] | Yellow solid | 71.1 | Decomposition 263-319 | 312.18 |
| 84 | 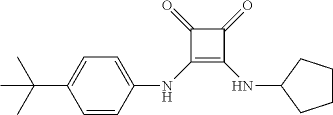 [SMILES: CC(C)(C)c1ccc(Nc2c(NC3CCCC3)c(=O)c2=O)cc1] | White solid | 80.7 | Decomposition 261-308 | 312.18 |
| 85 | 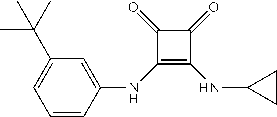 [SMILES: CC(C)(C)c1cccc(Nc2c(NC3CC3)c(=O)c2=O)c1] | White solid | 67.8 | Decomposition 171-239 | 284.15 |
| 86 | 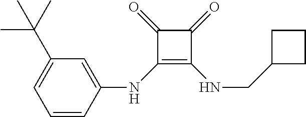 [SMILES: CC(C)(C)c1cccc(Nc2c(NCC3CCC3)c(=O)c2=O)c1] | White solid | 53.8 | Decomposition 234-241 | 312.18 |
| 87 | 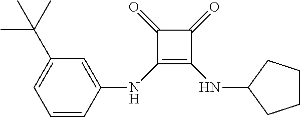 [SMILES: CC(C)(C)c1cccc(Nc2c(NC3CCCC3)c(=O)c2=O)c1] | White solid | 48.8 | Decomposition 239-247 | 312.18 |
| 88 | 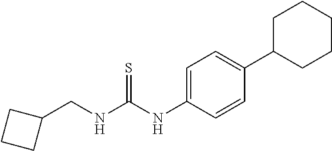 [SMILES: S=C(NCC1CCC1)Nc1ccc(C2CCCCC2)cc1] | Light brown solid | 71.6 | 144-152 | 302.18 |
| 89 | 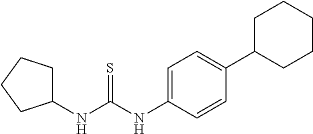 [SMILES: S=C(Nc1ccc(C2CCCCC2)cc1)NC1CCCC1] | White solid | 100.0 | 146-152 | 302.18 |
| 90 | 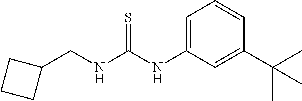 [SMILES: CC(C)(C)c1cccc(NC(=S)NCC2CCC2)c1] | White solid | 57.0 | 105-111 | 276.17 |
| 91 | 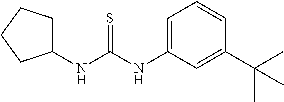 [SMILES: CC(C)(C)c1cccc(NC(=S)NC2CCCC2)c1] | White solid | 21.0 | 170-180 | 276.17 |
| 92 | 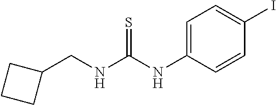 [SMILES: S=C(NCC1CCC1)Nc1ccc(I)cc1] | White solid | 18.0 | 98-108 | 346.00 |
| 93 | 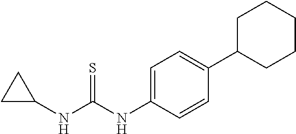 [SMILES: S=C(Nc1ccc(C2CCCCC2)cc1)NC1CC1] | Light yellow solid | 53.0 | 157-165 | 274.15 |
| 94 | 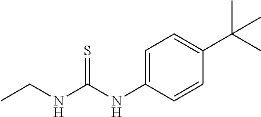 [SMILES: CCNC(=S)Nc1ccc(C(C)(C)C)cc1] | Light yellow solid | 74.0 | 109-115 | 236.13 |
| 95 | 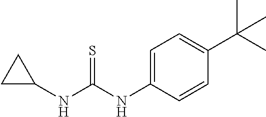 [SMILES: CC(C)(C)c1ccc(NC(=S)NC2CC2)cc1] | White solid | 100.0 | 119-143 | 248.13 |
| 96 | 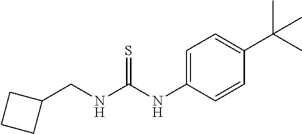 [SMILES: CC(C)(C)c1ccc(NC(=S)NCC2CCC2)cc1] | Light brown solid | 25.8 | 129-146 | 276.17 |
| 97 | 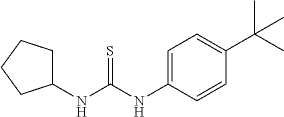 [SMILES: CC(C)(C)c1ccc(NC(=S)NC2CCCC2)cc1] | White solid | 73.2 | 142-154 | 276.17 |
| 98 | 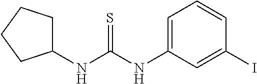 [SMILES: S=C(Nc1cccc(I)c1)NC1CCCC1] | White solid | 21.4 | 136-147 | 346.0 |
| 99 | 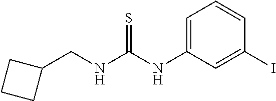 [SMILES: S=C(NCC1CCC1)Nc1cccc(I)c1] | White solid | 20.0 | 136-147 | 346.0 |
| 100 | 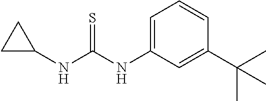 [SMILES: CC(C)(C)c1cccc(NC(=S)NC2CC2)c1] | White solid | 78.0 | 125-140 | 248.13 |
| 101 | 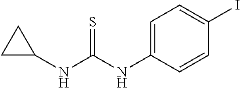 [SMILES: S=C(Nc1ccc(I)cc1)NC1CC1] | Yellow solid | 67.0 | 166-180 | 317.97 |
| 102 | 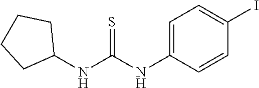 [SMILES: S=C(Nc1ccc(I)cc1)NC1CCCC1] | Light brown solid | 22.0 | 154-162 | 346.0 |
| 103 | 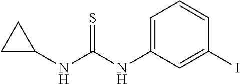 [SMILES: S=C(Nc1cccc(I)c1)NC1CC1] | White solid | 55.0 | 142-152 | 317.97 |
| 104 | 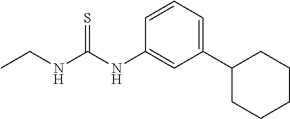 [SMILES: CCNC(=S)Nc1cccc(C2CCCCC2)c1] | Sticky pale yellow oil | 30.0 | | 262.15 |
| 105 | 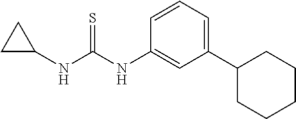 [SMILES: S=C(Nc1cccc(C2CCCCC2)c1)NC1CC1] | Light yellow solid | 100.0 | 122-128 | 274.15 |
| 106 | 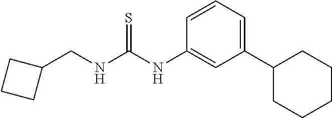 [SMILES: S=C(NCC1CCC1)Nc1cccc(C2CCCCC2)c1] | Light yellow solid | 51.0 | 102-110 | 302.18 |
| 107 | 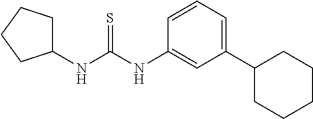 [SMILES: S=C(Nc1cccc(C2CCCCC2)c1)NC1CCCC1] | White solid | 62.0 | 129-142 | 302.18 |

TABLE 5
Effect on IL-6 expression and antiproliferative activity of compounds
1-18 (Table 4) on the HaCaT, HT-29, A549 and HDFn cell lines.

| | HaCaT | HT-29 | A549 | HDFn | Reduction |
|---|---|---|---|---|---|
| Compound | (IC₅₀) | (IC₅₀) | (IC₅₀) | (IC₅₀) | IL-6 (%) |
| 1 | 91.9 | 54.8 | 81.9 | 83.6 | −6.9 |
| 2 | 44.7 | 32.9 | 37.1 | 27.1 | −14.6 |
| 3 | 94.9 | 51.9 | 82.7 | 81.3 | 16.2 |
| 4 | 94.5 | >100 | 80.3 | 59.1 | 1.7 |
| 5 | >100 | 81.3 | 61.8 | 17.4 | 54.5 |
| 6 | 99.7 | 87.9 | 80.0 | 44.2 | 57.2 |
| 7 | >100 | 74.2 | 83.2 | 57.4 | 13.6 |
| 8 | 55.0 | 35.6 | 34.2 | 14.5 | −10.6 |
| 9 | >100 | 54.9 | 67.1 | 10.5 | 20.0 |
| 10 | >100 | 85.2 | 96.8 | 92.4 | 35.3 |
| 11 | 46.5 | 34.9 | 36.7 | 13.1 | −41.6 |
| 12 | >100 | >100 | >100 | 81.5 | −9.6 |
| 13 | >100 | 56.9 | 60.8 | 59.1 | 51.3 |
| 14 | 35.4 | 25.5 | 18.5 | 17.2 | 28.5 |
| 15 | >100 | >100 | >100 | 59.1 | 33.4 |
| 16 | >100 | >100 | >100 | 30.5 | 26.1 |
| 17 | 87.9 | 58.3 | 44.1 | 16.3 | 52.4 |
| 18 | 32.0 | 24.1 | 21.0 | 23.0 | 70.4 |

| | |
|---|---|
| Dexamethasone | 68.3 |
| DMSO | |
| DMSO + Cytokines | |
| Ibuprofen | 62.1 |

TABLE 6
Effect on IL-6 expression and antiproliferative activity of compounds
20, 23-24, 27, 30-31, 36, 39, 40, 44, 47-48, 54, 57-58, 63 and
66-69 (Table 4) on the HaCaT, HT-29, A549 and HDFn cell lines.

| | HaCaT | HT-29 | A549 | HDFn | Reduction |
|---|---|---|---|---|---|
| Compound | (IC₅₀) | (IC₅₀) | (IC₅₀) | (IC₅₀) | IL-6 (%) |
| 20 | 100 | 82 | 100 | 95 | 73.1 |
| 23 | 62 | 29 | 27 | 74 | 81.0 |
| 24 | 100 | 9.7 | 62 | 100 | 63.4 |
| 27 | 100 | 85 | 100 | 55 | 48.4 |
| 30 | 35 | 21 | 100 | 8.2 | 60.3 |
| 31 | 100 | 100 | 83 | 100 | 28.4 |
| 36 | 100 | 100 | 84 | 79 | 67.8 |
| 39 | 31 | 25 | 25 | 15 | 49.3 |
| 40 | 49 | 30 | 30 | 23 | 49.6 |
| 44 | 100 | 100 | 100 | 100 | 71.7 |
| 47 | 39 | 43 | 37 | 21 | 73.3 |
| 48 | 100 | 27 | 34 | 84 | 58.1 |
| 54 | 74 | 54 | 48 | 40 | 83.6 |
| 57 | 100 | 100 | 100 | 100 | 64.0 |
| 58 | 82 | 13 | 11 | 56 | 71.7 |
| 63 | 100 | 75 | 69 | 60 | 54.0 |
| 66 | 37 | 31 | 58 | 31 | 39.7 |
| 67 | 17 | 26 | 37 | 25 | 44.1 |
| 68 | 3.7 | 5.6 | 8.0 | 12 | 44.9 |
| 69 | 4.0 | 8.9 | 12 | 7.6 | 41.3 |

| | |
|---|---|
| IL-17α/TNFα | |
| DMSO | |
| Curcumin | 64.6 |
| Ibuprofen | 32.6 |
| Dexamethasone | 45.3 |
| (1 μM) | |

TABLE 7
Effect on IL-6 expression and antiproliferative activity of compounds
70-87 (Table 4) on the HaCaT, HT-29, A549 and HDFn cell lines.

| | HaCaT | HT-29 | A549 | HDFn | Reduction |
|---|---|---|---|---|---|
| Compound | (IC₅₀) | (IC₅₀) | (IC₅₀) | (IC₅₀) | IL-6 (%) |
| 70 | >100 | >100 | >100 | >100 | 0.9 |
| 71 | >100 | >100 | >100 | >100 | −1.4 |
| 72 | 10.6 | 10.0 | 8.0 | 6.9 | 50.0 |
| 73 | 37.7 | 39.8 | 26.8 | 19.6 | −3.2 |
| 74 | 10.9 | 15.0 | 8.3 | 4.1 | −54.2 |
| 75 | 11.8 | 15.2 | 9.0 | 4.6 | −19.3 |
| 76 | >100 | >100 | 74.0 | >100 | −18.7 |
| 77 | >100 | >100 | 89.6 | >100 | −19.7 |
| 78 | >100 | >100 | 96.1 | >100 | −14.3 |
| 79 | 11.0 | 7.9 | 8.0 | 4.3 | 6.7 |
| 80 | 6.0 | 4.5 | 4.2 | 1.9 | 11.8 |
| 81 | 5.6 | 4.3 | 3.7 | 1.9 | −34.2 |
| 82 | 47.9 | 18.7 | 17.9 | 13.2 | 10.4 |
| 83 | >100 | >100 | >100 | >100 | −9.5 |
| 84 | >100 | >100 | 15.1 | >100 | 12.1 |
| 85 | 81.4 | 64.1 | 40.8 | 57.3 | 41.7 |
| 86 | 56.1 | 14.8 | 13.7 | 3.8 | −103.7 |
| 87 | 5.5 | 4.3 | 4.0 | 2.8 | −58.6 |

| | |
|---|---|
| Dexamethasone | 68.3 |
| DMSO | |
| DMSO + Cytokines | |
| Ibuprofen | 62.1 |

TABLE 8
Effect on IL-6 expression and antiproliferative activity of compounds
20, 23-24, 27, 30-31, 36, 39, 40, 44, 47-48, 54, 57-58, 63 and
66-69 (Table 4) on the HaCaT, HT-29, A549 and HDFn cell lines.

| | HaCaT | HT-29 | A549 | HDFn | Reduction |
|---|---|---|---|---|---|
| Compound | (IC₅₀) | (IC₅₀) | (IC₅₀) | (IC₅₀) | IL-6 (%) |
| 88 | 32.5 | 20.4 | 15.3 | 15.7 | 1.8 |
| 89 | 34.9 | 21.6 | 20 | 14.6 | 4.8 |
| 90 | 34.3 | 20.2 | 17.5 | 18.2 | −27.9 |
| 91 | 31.8 | 21 | 19.4 | 16.3 | −12.0 |
| 92 | 48.2 | 27.2 | 26.2 | 24.2 | 9.6 |
| 93 | 84.7 | 30.8 | 20.8 | 84 | 22.1 |
| 94 | >100 | 92 | 71.1 | 94 | −18.2 |
| 95 | 94.5 | 64.4 | 57.9 | 60 | −28.5 |
| 96 | >100 | 44 | 23.3 | >100 | 6.8 |
| 97 | 40 | 28.8 | 26.2 | 22.6 | 8.6 |
| 98 | 32.8 | 23.4 | 21.9 | 16.4 | 36.8 |
| 99 | 35.5 | 27 | 26.2 | 19.4 | 49.2 |
| 100 | >100 | 75.7 | 70 | >100 | 46.5 |
| 101 | >100 | 63.3 | 65.1 | >100 | 23.8 |
| 102 | 39.1 | 25.1 | 25.3 | 18.6 | 45.5 |
| 103 | >100 | >100 | >100 | >100 | 9.6 |
| 104 | 19.3 | 17.9 | 22.4 | 12.9 | 15.2 |
| 105 | 16.3 | 12.7 | 15.9 | 14.7 | 44.1 |
| 106 | 14.9 | 13.2 | 17 | 4.9 | 48.4 |
| 107 | 7.9 | 5.7 | 6.2 | 3.8 | 42.9 |

| | |
|---|---|
| Curcumin | 32.3 |
| Dexamethasone | 54.5 |
| (1 μM) | |
| DMSO | |
| DMSO + Cytokines | |
| Ibuprofen | 51.0 |

IL-6 Evaluation

HaCaT cells (1×10⁵) were suspended in DMEM culture media (500 μL) and incubated for 24 h in 24-well microtiter plates at 37° C. in a moisture-saturated atmosphere containing 5% CO₂. Compounds were solubilized in DMSO and diluted in fresh DMEM. The compound (500 μL) was then added to the cell medium to obtain a final concentration of 10 μL/well and the cells incubated over a period of 1 hour. In the meantime, IL-17α (200 ng/mL) and TNFα (20 ng/mL) (PeproTech, Rocky Hill, N.J.) were added to the drug solution in DMEM. Following the initial incubation of the cells, the culture medium was aspirated from each well and the IL-17α+ TNFα+ compound solution was added to each well for incubation (6 hours). The culture media was then removed and transferred to a clean tube (1.5 mL) at −80° C. until the ELISA test was performed. The presence of IL-6 in the cell media was determined using an IL-6 human Duoset ELISA kit (Fisher Scientific, Ottawa, On.) according to the manufacturer's instructions. Standards and samples were prepared and assessed in duplicate. Two separate replicates were performed for each sample. The absorbance was measured at 450 nm and 540 nm using a TECAN infinite M1000 plate reader.

Antiproliferative Activity

The antiproliferative activity assay of all compounds was assessed using the procedure recommended by the National Cancer Institute for its drug screening program with minor modifications. Briefly, 96-well microtiter plates were seeded with 75 μL of a suspension of either HaCaT (5×10³), HT-29 (3.0×10³), A549 (3.0×10³) or HDFn (3×10³) cells per well in DMEM and incubated for 24 h at 37° C. in a moisture-saturated atmosphere containing 5% CO₂. Compounds freshly solubilized in DMSO (40 mM) were diluted in fresh DMEM, and 75 μL aliquots containing serially diluted concentrations of the compound were added. Final compound concentrations ranged from 100 μM to 781 nM. DMSO was maintained at a concentration of <0.5% (v/v) to avoid any related cytotoxicity. Plates were subsequently incubated for 48 h. Cell growth was then stopped by the addition of cold trichloroacetic acid to the wells (10% w/v, final concentration), followed by a 1 h incubation period at 4° C. The plates were washed 4-times with water. A sulforhodamine B solution (75 μL; 0.1% w/v) in acetic acid (1%) was subsequently added to each well and the plates incubated over a 15 min period while at room temperature. After staining, any unbound dye was removed by washing 4-times with an acetic acid solution (1%). Bound dye was solubilized in 20 mM Tris base and the absorbance was measured at an optimal wavelength (530-568 nm) using a μQuant® Universal microplate spectrophotometer (BioTek, Winooski, Vt.). The measurements for treated cells were compared with measurements from control cell plates fixed on treatment day and the percentage of cell growth inhibition was calculated for each drug. The experiments were performed at least twice in triplicate. The assays were considered valid when the coefficient of variation for a given set of conditions and within the same experiment was <10%.

Cell Cycle Analysis

HaCaT cells (2.5×10⁵) were incubated with compounds 3a-b, 4e, 5b-c, 6a and 6e and curcumin (10 μM) over a period of 24 h. The cells were subsequently trypsinized, washed with PBS, resuspended in PBS (250 μL), fixed by the addition of ice-cold EtOH (750 μL) under agitation and stored at −20° C. until analysis. Prior to FACS analysis, cells were washed with PBS and resuspended in PBS (500 μL) containing 4′,6′-diamidino-2-phenylindole (DAPI) (2 μg/mL). The cell cycle was analyzed using an LSR II flow cytometer (BD Biosciences, Franklin Lakes, N.J.).

Psoriasis (Imiquimod) in Mice

On day 0, Balb/c mice, aged 7 to 9 weeks, are shaved (¾ of the back) to clear the base of the neck. On day 1, the mice are topically treated on the shaved are with freshly prepared compound 20 (Table 4) at 2.5 mg/mouse in DMSO, dexamethasone at 0.2 mg/mouse in DMSO or with DMSO. After 1 hour, 62.5 mg of Imiquimod 5% (Apo-imiquimod) or base cream (Base Atlas Cream; negative control) is applied on the shaved area of the mice. Two hours later, the mice were treated a second time with compound 20 at 2.5 mg/mouse in DMSO, dexamethasone at 0.2 mg/mouse in DMSO or with DMSO. These treatments were repeated daily for a total of 6 days. The mice were weighed and the treated skin areas analyzed for redness, thickening and peeling (repeated daily). The mice were sacrificed on day 6 and the skin from the treated areas removed, fixed and embedded in paraffin for histological analysis. Organs, such as the liver, the kidneys and the spleen were harvested and fixed for future analysis.

Candidate compounds of Formula I-VI can be tested in vitro and/or in vivo to determine their activity in attenuating, inhibiting or preventing conditions associated with the expression of IL-6.

### EXPERIMENTAL

General: ¹H and ¹³C NMR spectra were recorded on a Bruker AM-300 spectrometer (Bruker, Germany). Chemical shifts (6) are reported in parts per million (ppm). Melting points were determined using an electrothermal melting point apparatus. HPLC analyses were performed using a Prominence LCMS-2020 system with binary solvent and equipped with an UV/vis photodiode array and an APCI probe (Shimadzu, Columbia, Md.). Compounds were eluted within 15 min on an Alltech® Alltima C18 reversed-phase column (5 mm, 250 mm, 4.6 mm) equipped with an Alltech® Alltima C18 pre-column (5 mm, 7.5 mm, 4.6 mm) and a MeOH/H₂O linear gradient of 60:40 at 1.0 mL/min. The purities of the final compounds were >95%. All chemicals were supplied by Sigma-Aldrich Canada (Oakville, Ontario, Canada), VWR International (Mont-Royal, Québec, Canada) or Enamine LLC (Princeton, N.J., USA) and used as received unless specified otherwise. Flash column chromatography was performed on silica gel F60, 60 Å, 40-63 μm supplied by Silicycle (Quebec City, Québec, Canada) using a FPX flash purification system (Biotage, Charlottesville, Va.) and using solvent mixtures expressed as volume/volume ratios. The progress of the chemical reactions was monitored by TLC using pre-coated silica gel 60 F254 TLC plates (VWR International, Mont-Royal, Québec, Canada). The chromatograms and spots were visualized under UV light at 254 and/or 265 nm.

A number of non-limiting examples, illustrating the preparation of selected substituted phenyl cycloalkylureas in accordance with the present disclosure, are illustrated in the following section.

General Procedure for the Preparation of tBCEU, cHCEU, and 3a-b, 4a-e, 5a-e and 6a-e.

Anilines 1a-1e (1.0 eq.) were dissolved in acetonitrile (6 ml) and K₂CO₃ (1.2 eq.) was added to the resulting solution. The proper isocyanate (2a-2d; 1.2 eq.) was then added to the solution and the reaction mixture was stirred for 48 h under reflux. The reaction mixture was then evaporated to dryness under reduced pressure and the resulting residue was purified by flash chromatography on silica gel.

1-(4-(t-Butyl)phenyl)-3-ethylurea (3a). Flash chromatography (hexanes/ethyl acetate (80:20)) Yield: 73%; White solid; mp: 136-139° C.; ¹H NMR (CDCl₃ and MeOD): δ 7.18-7.08 (m, 4H, Ar), 3.10 (quint, 2H, J=6.6 Hz, CH₂), 1.18-1.12 (m, 9H, 3× CH₃), 1.03-0.94 (m, 3H, CH₃); ¹³C NMR (CDCl₃ and MeOD): δ 156.9, 145.5, 136.4, 125.6, 119.4, 34.5, 34.0, 31.2, 15.1. MS (ES+) found 221.20; C₁₃H₂₀N₂O (M⁺+H) requires 221.17.

1-(4-(t-Butyl)phenyl)-3-cyclopropylurea (4a). Flash chromatography (hexanes/ethyl acetate (80:20)) Yield: 28%; Light yellow solid; mp: 138-139° C.; ¹H NMR (CDCl₃): δ 7.29-7.27 (m, 4H, Ar), 7.21 (s, 1H, NH), 5.42 (s, 1H, NH), 2.62-2.52 (m, 1H, CH), 1.28 (s, 9H, 3× CH₃), 0.76 (d, 2H, J=5.5 Hz, 2× CH), 0.57 (s, 2H, 2× CH); ¹³C NMR (CDCl₃): δ 157.4, 146.4, 135.9, 125.9, 120.2, 34.3, 31.4, 22.6, 7.4. MS (ES+) found 233.20; C₁₄H₂₀N₂O (M⁺+H) requires 233.17.

1-Cyclopropyl-3-(4-iodophenyl)urea (4b). Flash chromatography (hexanes/ethyl acetate (75:25)) Yield: 20%; White solid; mp:208-209° C.; ¹H NMR (CDCl₃ and MeOD): δ 7.42 (d, 2H, J=8.3 Hz, Ar), 7.06 (d, 2H, J=8.3 Hz, Ar), 2.44 (apparent non, 1H, CH), 0.65-0.58 (m, 2H, 2× CH), 0.43-0.37 (m, 2H, 2× CH); ¹³C NMR (CDCl₃ and MeOD): δ 157.2, 138.9, 137.5, 120.9, 84.9, 22.1, 6.7. MS (ES+) found 303.00; C₁₀H₁₁IN₂O (M⁺+H) requires 333.05.

1-(3-t-Butyl)phenyl)-3-cyclopropylurea (4c). Flash chromatography (hexanes/ethyl acetate (80:20)) Yield: 34%; White solid; mp: 169-171° C.; ¹H NMR (CDCl₃ and MeOD): δ 7.36-7.34 (m, 1H, Ar), 7.19-7.16 (m, 2H, Ar), 7.07-7.04 (m, 1H, Ar), 2.58-2.52 (m, 1H, CH), 1.27 (s, 9H, 3× CH₃) 0.78-0.72 (m, 2H, 2× CH), 0.58-0.54 (m, 2H, 2× CH); ¹³C NMR (CDCl₃ and MeOD): δ 157.3, 152.2, 138.1, 128.6, 120.5, 117.4, 34.7, 31.2, 22.4, 7.2. MS (ES+) found 233.10; C₁₄H₂₀N₂O (M⁺+H) requires 233.17.

1-Cyclopropyl-3-(3-iodophenyl)urea (4d). Flash chromatography (hexanes/ethyl acetate (80:20)) Yield: 27%; White solid; mp: 153-154° C.; ¹H NMR (CDCl₃ and MeOD): δ 7.74-7.72 (m, 1H, Ar), 7.30-7.25 (m, 2H, Ar), 6.92 (t, 1H, J=8.0 Hz, Ar), 2.54-2.47 (m, 1H, CH), 0.70 (d.d, 2H, J=6.0 Hz, 2× CH), 0.51-0.46 (m, 2H, 2× CH); ¹³C NMR (CDCl₃ and MeOD): δ 157.0, 140.1, 131.6, 130.3, 127.8, 118.4, 94.1, 22.3, 7.0. MS (ES+) found 303.00; C₁₀H₁₁IN₂O (M⁺+H) requires 303.00.

1-(4-Cyclohexylphenyl)-3-cyclopropylurea (4e). Flash chromatography (hexanes/ethyl acetate (80:20)) Yield: 66%; White solid; mp: 173-176° C.; ¹H NMR (CDCl₃ and MeOD): δ 7.21 (d, 2H, J=8.4 Hz, Ar), 7.07 (d, 2H, J=8.4 Hz, Ar), 2.51 (apparent sept, 1H, CH), 2.44-2.32 (m, 1H, CH), 1.79-1.19 (m, 10H, 5× CH₂), 0.71 (q, 2H, J=6.8 Hz, 2× CH), 0.54-0.49 (m, 2H, 2× CH); ¹³C NMR (CDCl₃ and MeOD): δ 157.5, 143.4, 136.1, 127.3, 120.3, 43.9, 34.5, 26.9, 26.1, 22.4, 7.1. MS (ES+) found 259.20; C₁₆H₂₂N₂O (M⁺+H) requires 259.18.

1-(4-(t-Butyl)phenyl)-3-(cyclobutylmethyl)urea (5a). Flash chromatography (hexanes/ethyl acetate (80:20)) Yield: 19%; White solid; mp: 179-184° C.; ¹H NMR (CDCl₃): δ 7.30 (d, 2H, J=8.7 Hz, Ar), 7.18 (d, 2H, J=8.7 Hz, Ar), 3.23 (d, 2H, J=7.2 Hz, CH₂), 2.44 (sept, 1H, J=7.5 Hz, CH), 2.06-1.94 (m, 2H, 2× CH), 1.91-1.78 (m, 2H, 2× CH), 1.71-1.60 (m, 2H, CH₂) 1.30-1.27 (m, 9H, 3× CH₃); ¹³C NMR (CDCl₃): δ 156.8, 147.1, 135.5, 126.1, 121.4, 45.6, 35.3, 34.3, 31.3, 25.6, 18.2. MS (ES+) found 261.20; C₁₆H₂₄N₂O (M⁺+H) requires 261.20.

1-(Cyclobutylmethyl)-3-(4-iodophenyl)urea (5b). Flash chromatography (hexanes/ethyl acetate (80:20)) Yield: 10%; White solid; mp: 159-160° C.; ¹H NMR (CDCl₃ and DMSO-d₆): δ 7.35 (d, 2H, J=8.9 Hz, Ar), 7.07 (d, 2H, J=8.9 Hz, Ar), 3.08 (d, 2H, J=7.1 Hz, CH₂), 2.33 (sept, 1H, J=7.5 Hz, CH), 1.85-1.93 (m, 2H, 2× CH), 1.77-1.69 (m, 2H, 2× CH), 1.61-1.53 (m, 2H, CH₂); ¹³C NMR (CDCl₃ and DMSO-d₆): δ 155.9, 140.1, 137.4, 120.2, 83.6, 44.8, 35.4, 25.5, 18.2. MS (ES+) found 331.00; C₁₂H₁₅IN₂O (M⁺+H) requires 331.03.

1-(3-(t-Butyl)phenyl)-3-(cyclobutylmethyl)urea (5c). Flash chromatography (hexanes/ethyl acetate (90:10)) Yield: 55%; White solid; mp: 126-127° C.; ¹H NMR (CDCl₃ and MeOD): δ 7.32-7.30 (m, 1H, Ar), 7.18-7.00 (m, 3H, Ar), 3.18 (d, 2H, J=7.2 Hz, CH₂), 2.39 (sept, 1H, J=7.5 Hz, CH), 2.02-1.92 (m, 2H, 2× CH), 1.88-1.76 (m, 2H, CH₂), 1.68-1.59 (m, 2H, 2× CH), 1.24 (s, 9H, 3× CH₃); ¹³C NMR (CDCl₃ and MeOD): δ 156.9, 152.3, 138.6, 128.6, 120.3, 117.5, 117.5, 45.2, 35.3, 34.6, 31.2, 25.5, 18.2. MS (ES+) found 261.25; C₁₆H₂₄N₂O (M⁺+H) requires 261.20.

1-(Cyclobutylmethyl)-3-(3-iodophenyl)urea (5d). Flash chromatography (hexanes/ethyl acetate (90:10)) Yield: 45%; White solid; mp: 138-140° C.; ¹H NMR (CDCl₃ and MeOD): δ 7.66-7.63 (m, 1H, Ar), 7.18-7.11 (m, 2H, Ar), 6.85-6.77 (m, 1H, Ar), 3.09-3.04 (m, 2H, CH₂), 2.38-2.26 (m, 1H, CH), 1.96-1.85 (m, 2H, 2× CH), 1.79-1.68 (m, 2H, 2× CH), 1.61-1.51 (m, 2H, CH₂); ¹³C NMR (CDCl₃ and MeOD): δ 156.2, 140.9, 130.9, 130.2, 127.2, 117.7, 94.0, 44.8, 35.2, 25.3, 18.1. MS (ES+) found 331.00; C₁₂H₁₅IN₂O (M⁺+H) requires 331.03.

1-(Cyclobutylmethyl)-3-(4-cyclohexylphenyl)urea (5e). Flash chromatography (hexanes/ethyl acetate (80:20)) Yield: 32%; White solid; mp: 172-173° C.; ¹H NMR (CDCl₃ and MeOD): δ 7.17 (d, 2H, J=8.4 Hz, Ar), 7.08 (d, 2H, J=8.5 Hz, Ar), 3.20-3.17 (m, 2H, CH₂), 2.46-2.37 (m, 2H, 2× CH), 2.03-1.94 (m, 2H, CH₂), 1.88-1.20 (m, 14H, 7× CH₂); ¹³C NMR (CDCl₃ and MeOD): δ 156.9, 143.2, 136.5, 127.3, 120.6, 45.1, 43.9, 35.4, 34.5, 26.9, 26.1, 25.5, 18.2. MS (ES+) found 287.25; C₁₈H₂₆N₂O (M⁺+H) requires 287.21.

1-(4-(t-Butyl)phenyl)-3-cyclopentylurea (6a). Flash chromatography (hexanes/ethyl acetate (80:20)) Yield: 15%; White solid; mp: 219-221° C.; ¹H NMR (CDCl₃ and MeOD): δ 7.21 (d, 2H, J=9.0 Hz, Ar), 7.15 (d, 2H, J=8.9 Hz, Ar), 3.99 (apparent quint, 1H, J=6.7 Hz, CH), 1.93-1.82 (m, 2H, CH₂), 1.64-1.45 (m, 4H, 2× CH₂), 1.37-1.25 (m, 2H, CH₂) 1.21 (s, 9H, 3× CH₃); ¹³C NMR (CDCl₃ and MeOD): δ 156.5, 145.8, 136.2, 125.8, 119.7, 51.6, 34.1, 33.2, 31.3, 23.5. MS (ES+) found 261.25; C₁₆H₂₄N₂O (M⁺+H) requires 261.20.

1-Cyclopentyl-3-(4-iodophenyl)urea (6b). Flash chromatography (hexanes/ethyl acetate (80:20)) Yield: 13%; White solid; mp: 188-191° C.; ¹H NMR (CDCl₃ and DMSO-d₆): δ 7.31 (d, 2H, J=8.7 Hz, Ar), 7.03 (d, 2H, J=8.8 Hz, Ar), 3.93-3.87 (m, 1H, CH), 1.81-1.73 (m, 2H, CH₂), 1.53-1.39 (m, 4H, 2× CH₂), 1.27-1.19 (m, 2H, CH₂); ¹³C NMR (CDCl₃ and DMSO-d₆): δ 155.4, 140.1, 137.3, 120.1, 83.4, 51.3, 33.3, 23.5. MS (ES+) found 331.00; C₁₂H₁₅IN₂O (M⁺+H) requires 331.03.

1-(3-(t-Butyl)phenyl)-3-cyclopentylurea (6c). Flash chromatography (hexanes/ethyl acetate (90:10)) Yield: 57%; White solid; mp: 152-154° C.; ¹H NMR (CDCl₃ and MeOD): δ 7.33-7.31 (m, 1H, Ar), 7.16-7.13 (m, 1H, Ar), 7.09-7.01 (m, 2H, Ar), 4.05 (quint, 1H, J=6.8 Hz, CH), 1.96-1.85 (m, 2H, 2× CH), 1.63-1.48 (m, 4H, 2× CH₂), 1.38-1.29 (m, 2H, 2× CH), 1.25 (s, 9H, 3× CH₃); ¹³C NMR (CDCl₃ and MeOD): δ 156.3, 152.3, 138.6, 128.6, 120.2, 117.4, 117.3, 51.7, 34.6, 33.3, 31.2, 23.5. MS (ES+) found 261.25; C₁₆H₂₄N₂O (M⁺+H) requires 261.20.

1-Cyclopentyl-3-(3-iodophenyl)urea (6d). Flash chromatography (hexanes/ethyl acetate (90:10)) Yield: 16%; White solid; mp: 142-143° C.; ¹H NMR (CDCl₃ and MeOD): δ 7.71-7.69 (m, 1H, Ar), 7.28-7.21 (m, 2H, Ar), 6.90 (t, 1H, J=8.0 Hz, Ar), 3.99 (apparent quint, 1H, J=6.5 Hz, CH), 1.93-1.86 (m, 2H, 2× CH), 1.61-1.52 (m, 4H, 4× CH) 1.37-1.30 (m, 2H, 2× CH); ¹³C NMR (CDCl₃ and MeOD): δ 155.7, 140.8, 131.1, 130.3, 127.4, 117.9, 94.1, 51.5, 33.2, 23.5. MS (ES+) found 331.00; C₁₂H₁₅IN₂O (M⁺+H) requires 331.03.

1-(4-Cyclohexylphenyl)-3-cyclopentylurea (6e). Flash chromatography (hexanes/ethyl acetate (80:20)) Yield: 30%; White solid; mp: 197-199° C.; ¹H NMR (CDCl₃ and MeOD): δ 7.14 (d, 2H, J=8.5 Hz, Ar), 7.05 (d, 2H, J=8.4 Hz, Ar), 4.00 (quint, 1H, J=6.6 Hz, CH), 2.42-2.32 (m, 1H, CH), 1.95-1.85 (m, 2H, 2× CH), 1.81-1.20 (m, 16H, 2× CH+7× CH₂); ¹³C NMR (CDCl₃ and MeOD): δ 156.4, 143.1, 136.6, 127.3, 120.3, 51.7, 43.9, 34.5, 33.3, 26.9, 26.1, 23.5. MS (ES+) found 287.20; C₁₈H₂₆N₂O (M⁺+H) requires 287.21.

General Procedure for the Preparation of 7-38.

Suitable aniline (1.0 eq.) were dissolved in acetonitrile (6 ml) and K₂CO₃ (1.2 eq.) was added to the resulting solution. The proper isocyanate (1.2 eq.) was then added to the solution and the reaction mixture was stirred for 48 h under reflux. The reaction mixture was then evaporated to dryness under reduced pressure and the resulting residue was purified by flash chromatography on silica gel.

1-(4-Iodophenyl)-3-neopentylurea (7). Flash chromatography (methylene chloride/hexanes (95:5)) Yield: 11%; Sticky solid; ¹H NMR (CDCl₃ and DMSO-d₆): δ 7.45 (d, 2H, J=8.9 Hz, Ar), 7.14 (d, 2H, J=8.9 Hz, Ar), 2.97 (s, 2H, CH₂), 0.87 (s, 9H, 3× CH₃); ¹³C NMR (CDCl₃ and DMSO-d₆): δ 156.1, 140.0, 137.5, 120.6, 84.0, 51.1, 31.9, 27.2. MS (ES+) found 333.00; C₁₂H₁₇IN₂O (M⁺+H) requires 333.05.

1-(Cyclopropylmethyl)-3-(4-iodophenyl)urea (8). Flash chromatography (hexanes/ethyl acetate (80:20)) Yield: 26%; White solid; mp: 192-194° C.; ¹H NMR (CDCl₃ and MeOD): δ 7.42 (d, 2H, J=8.6 Hz, Ar), 7.07 (d, 2H, J=8.6 Hz, Ar), 2.97 (apparent t, 2H, J=7.1 Hz, CH₂), 0.93-0.82 (m, 1H, CH), 0.44-0.35 (m, 2H, 2× CH), 0.15-0.06 (m, 2H, 2× CH); ¹³C NMR (CDCl₃ and MeOD): δ 156.1, 139.5, 137.5, 120.6, 84.3, 44.4, 10.9, 3.1. MS (ES+) found 317.00; C₁₁H₁₃IN₂O (M⁺+H) requires 317.02.

1-Cyclobutyl-3-(4-iodophenyl)urea (9). Flash chromatography (hexanes/ethyl acetate (90:10)) Yield: 13%; White solid; mp: 208-210° C.; ¹H NMR (CDCl₃ and DMSO-d₆): δ 7.35 (d, 2H, J=8.9 Hz, Ar), 7.06 (d, 2H, J=8.9 Hz, Ar), 4.12 (quint, 1H, J=7.9 Hz, CH), 2.23-2.14 (m, 2H, 2× CH), 1.77-1.64 (m, 2H, 2× CH), 1.60-1.50 (m, 2H, CH₂); ¹³C NMR (CDCl₃ and DMSO-d₆): δ 154.7, 139.9, 137.4, 120.3, 83.8, 45.0, 31.6, 14.8. MS (ES+) found 317.00; C₁₁H₁₃IN₂O (M⁺+H) requires 317.02.

1-(Cyclopentylmethyl)-3-(4-iodophenyl)urea (10). Flash chromatography (hexanes/ethyl acetate (80:20)) Yield: 10%; White solid; mp: 158-162° C.; ¹H NMR (CDCl₃ and DMSO-d₆): δ 7.33 (d, 2H, J=8.9 Hz, Ar), 7.05 (d, 2H, J=8.9 Hz, Ar), 2.97 (d, 2H, J=7.2 Hz, CH₂), 1.87 (sept, 1H, J=7.5 Hz, CH), 1.64-1.53 (m, 2H, 2× CH), 1.51-1.33 (m, 4H, 4× CH), 1.10-1.00 (m, 2H, 2× CH); ¹³C NMR (CDCl₃ and DMSO-d₆): δ 155.8, 140.2, 137.3, 120.1, 83.4, 44.5, 40.0, 30.2, 25.1. MS (ES+) found 345.00; C₁₃H₁₇IN₂O (M⁺+H) requires 345.05.

1-(4-Iodophenyl)-3-(2-methoxyethyl)urea (11). Flash chromatography (hexanes/ethyl acetate (80:20)) Yield: 20%; White solid; mp: 141-143° C.; ¹H NMR (CDCl₃): δ 7.56 (d, 2H, J=8.5 Hz, Ar), 7.09 (d, 2H, J=8.6 Hz, Ar), 3.55-3.47 (m, 2H, CH₂), 3.46-3.40 (m, 2H, CH₂), 3.38 (s, 3H, CH₃); ¹³C NMR (CDCl₃): δ 156.0, 138.8, 137.9, 121.8, 85.9, 72.4, 58.8, 40.5. MS (ES+) found 320.95; C₁₀H₁₃IN₂O₂ (M⁺+H) requires 321.01.

1-(4-(t-Butyl)phenyl)-3-(cyclopropylmethyl)urea (12). Flash chromatography (hexanes/ethyl acetate (80:20)) Yield: 17%; White solid; mp: 168-170° C.; ¹H NMR (CDCl₃ and DMSO-d₆): δ 7.21-7.20 (m, 4H, Ar), 3.03 (d, 2H, J=7.0 Hz, CH₂), 1.22-1.21 (m, 9H, 3× CH₃), 0.93-0.86 (m, 1H, CH), 0.44-0.36 (m, 2H, 2× CH), 0.15-0.11 (m, 2H, 2× CH); ¹³C NMR (CDCl₃ and DMSO-d₆): δ 156.4, 145.3, 136.7, 125.7, 119.3, 44.7, 34.1, 31.4, 11.2, 3.3. MS (ES+) found 247.15; C₁₅H2₂N₂O (M⁺+H) requires 247.18.

1-(4-(t-Butyl)phenyl)-3-cyclobutylurea (13). Flash chromatography (hexanes/ethyl acetate (80:20)) Yield: 29%; White solid; mp: 189-190° C.; ¹H NMR (CDCl₃ and DMSO-d₆): δ 7.19-7.12 (m, 4H, Ar), 4.23-4.14 (m, 1H, CH), 2.27-2.15 (m, 2H, 2× CH), 1.79-1.65 (m, 2H, 2× CH), 1.61-1.49 (m, 2H, CH₂), 1.21-1.17 (m, 9H, 3× CH₃); ¹³C NMR (CDCl₃ and DMSO-d₆): δ 155.5, 140.8, 136.8, 125.6, 119.3, 45.2, 34.1, 31.6, 31.4, 14.8. MS (ES+) found 247.15; C₁₅H2₂N₂O (M⁺+H) requires 247.18.

1-(4-(t-Butyl)phenyl)-3-(2-methoxyethyl)urea (14). Flash chromatography (hexanes/ethyl acetate (80:20)) Yield: 29%; Light yellow solid; mp: 122-124° C.; ¹H NMR (CDCl₃): δ 7.29 (d, 2H, J=7.4 Hz, Ar), 7.21 (d, 2H, J=7.6 Hz, Ar), 5.62 (s, 1H, NH), 3.49-3.45 (m, 4H, 2× CH₂), 3.35 (s, 3H, CH₃), 1.29 (s, 9H, 3× CH₃); ¹³C NMR (CDCl₃): δ 146.6, 136.2, 126.1, 120.8, 72.4, 58.9, 40.3, 34.4, 31.5. MS (ES+) found 251.15; C₁₄H₂₂N₂O₂ (M⁺+H) requires 251.18.

1-(4-(t-Butyl)phenyl)-3-(pentan-3-yl)urea (15). Flash chromatography (hexanes/ethyl acetate (80:20)) Yield: 21%; White solid; mp: 119-121° C.; ¹H NMR (CDCl₃): δ 7.29 (d, 2H, J=8.6 Hz, Ar), 7.20 (d, 2H, J=8.6 Hz, Ar), 6.90 (s, 1H, NH), 4.93 (s, 1H, NH), 3.65 (apparent quint, 1H, J=7.9 Hz, CH), 1.58-1.45 (m, 2H, 2× CH) 1.41-1.19 (m, 11H, 2× CH+3× CH₃), 0.89 (t, 6H, J=7.4 Hz, 2× CH₃); ¹³C NMR (CDCl₃): δ 156.4, 146.6, 136.2, 126.1, 120.9, 52.9, 34.3, 31.4, 27.8, 10.3. MS (ES+) found 263.25; C₁₆H₂₆N₂O (M⁺+H) requires 263.21.

1-(Cyclopent-3-en-1-yl)-3-(4-iodophenyl)urea (16). Flash chromatography (hexanes/ethyl acetate (80:20)) Yield: 15%; White solid; mp: 201-202° C.; ¹H NMR (CDCl₃ and DMSO-d₆): δ 7.42 (d, 2H, J=8.8 Hz, Ar), 7.05 (d, 2H, J=8.6 Hz, Ar), 5.61-5.57 (m, 2H, CH₂), 4.34-4.26 (m, 1H, CH), 2.68-2.59 (m, 2H, 2× CH), 2.13-2.03 (m, 2H, 2× CH); ¹³C NMR (CDCl₃ and DMSO-d₆): δ 155.3, 140.1, 137.3, 128.9, 120.0, 83.4, 49.0, 40.3. MS (ES+) found 329.00; C₁₂H₁₃N₂O (M⁺+H) requires 329.02.

1-(Cyclopropylmethyl)-3-(3-iodophenyl)urea (17). Flash chromatography (hexanes/ethyl acetate (80:20)) Yield: 15%; White solid; mp: 139-140° C.; ¹H NMR (CDCl₃ and MeOD): δ 7.74-7.67 (m, 1H, Ar), 7.30-7.18 (m, 2H, Ar), 6.93-6.84 (m, 1H, Ar), 3.02-2.93 (m, 2H, CH₂), 0.94-0.82 (m, 2H, 2× CH), 0.16-0.05 (m, 2H, 2× CH); ¹³C NMR (CDCl₃ and MeOD): δ 156.0, 140.7, 130.9, 130.1, 127.2, 117.7, 93.9, 44.3, 10.7, 2.9. MS (ES+) found 317.00; C₁₁H₁₃IN₂O (M⁺+H) requires 317.02.

1-Cyclobutyl-3-(3-iodophenyl)urea (18). Flash chromatography (hexanes/ethyl acetate (80:20)) Yield: 16%; White solid; mp: 178-180° C.; ¹H NMR (CDCl₃ and MeOD): δ 7.68-7.62 (m, 1H, Ar), 7.22-7.12 (m, 2H, Ar), 6.88-6.79 (m, 1H, Ar), 4.09 (apparent sept, 1H, J=8.3 Hz, CH), 2.26-2.11 (m, 2H, 2× CH), 1.78-1.48 (m, 4H, 2× CH+CH₂); ¹³C NMR (CDCl₃ and MeOD): δ 155.1, 140.7, 131.0, 130.2, 127.3, 117.8, 94.0, 44.9, 31.4, 14.8. MS (ES+) found 317.00; C₁₁H₁₃IN₂O (M⁺+H) requires 317.02.

1-(Cyclopent-3-en-1-yl)-3-(3-iodophenyl)urea (19). Flash chromatography (hexanes/ethyl acetate (90:10)) Yield: 29%; Light brown solid; mp: 151-153° C.; ¹H NMR (CDCl₃ and MeOD): δ 7.69-7.67 (m, 1H, Ar), 7.25-7.21 (m, 2H, Ar), 6.89 (t, 1H, J=8.0 Hz, Ar), 4.34 (apparent sept, 1H, J=4.1 Hz, CH), 2.68 (dd, 2H, J=7.7 Hz, 2× CH), 2.13 (dd, 2H, 2× CH); ¹³C NMR (CDCl₃ and MeOD): δ 155.7, 140.7, 131.2, 130.3, 128.8, 127.5, 118.0, 94.2, 49.3, 40.3. MS (ES+) found 329.05; C₁₂H₁₃IN₂O (M⁺+H) requires 329.02.

1-(3-Iodophenyl)-3-(2-methoxyethyl)urea (20). Flash chromatography (hexanes/ethyl acetate (80:20)) Yield: 16%; Light yellow solid; mp: 99-101° C.; ¹H NMR (CDCl₃ and MeOD): δ 7.73 (s, 1H, Ar), 7.30 (d, 2H, J=7.8 Hz, Ar), 6.95 (t, 1H, J=7.7 Hz, Ar), 3.52-3.36 (m, 4H, 2× CH₂), 3.36 (s, 3H, CH₃); ¹³C NMR (CDCl₃ and MeOD): δ 156.1, 140.7, 131.2, 130.3, 127.5, 118.0, 94.1, 72.1, 58.7, 39.7. MS (ES+) found 321.00; C₁₀H₁₃IN₂O₂ (M⁺+H) requires 321.01.

1-(3-Iodophenyl)-3-neopentylurea (21). Flash chromatography (hexanes/ethyl acetate (90:10)) Yield: 45%; White solid; mp: 114-115° C.; ¹H NMR (CDCl₃ and MeOD): δ 7.68-7.66 (m, 1H, Ar), 7.18-7.12 (m, 2H, Ar), 6.85-6.77 (m, 1H, Ar), 2.87-2.85 (m, 2H, CH₂), 0.78-0.76 (m, 9H, 3× CH₃); ¹³C NMR (CDCl₃ and MeOD): δ 156.4, 141.0, 130.9, 130.2, 127.2, 117.7, 94.0, 50.9, 31.8, 26.9. MS (ES+) found 333.05; C₁₂H₁₇IN₂O (M⁺+H) requires 333.05.

1-(3-(t-Butyl)phenyl)-3-(cyclopropylmethyl)urea (22). Flash chromatography (hexanes/ethyl acetate (80:20)) Yield: 44%; White solid; mp: 165-166° C.; ¹H NMR (CDCl₃ and MeOD): δ 7.33-7.31 (m, 1H, Ar), 7.21-7.15 (m, 1H, Ar), 7.11-7.02 (m, 2H, Ar), 3.05 (d, 2H, CH₂), 1.26 (s, 9H, 3× CH₃) 0.97-0.87 (m, 1H, CH), 0.44-0.41 (m, 2H, 2× CH), 0.16-0.12 (m, 2H, 2× CH); ¹³C NMR (CDCl₃ and MeOD): δ 156.5, 152.3, 138.5, 128.7, 120.4, 117.7, 117.6, 44.8, 34.6, 31.2, 11.0, 3.1. MS (ES+) found 247.15; C₁₅H₂₂N₂O (M⁺+H) requires 247.18.

1-(3-(t-Butyl)phenyl)-3-cyclobutylurea (23). Flash chromatography (hexanes/ethyl acetate (80:20)) Yield: 57%; White solid; mp: 165-166° C.; ¹H NMR (CDCl₃ and MeOD): δ 7.34-7.32 (m, 1H, Ar), 7.18-7.08 (m, 2H, Ar), 7.05-7.01 (m, 1H, Ar), 4.23 (quint, 1H, J=7.5 Hz, CH), 2.31-2.21 (m, 2H, 2× CH), 1.83-1.69 (m, 2H, 2× CH), 1.64-1.53 (m, 2H, CH₂), 1.25 (s, 9H, 3× CH₃); ¹³C NMR (CDCl₃ and MeOD): δ 155.7, 152.2, 138.6, 128.6, 120.2, 117.5, 117.4, 45.2, 34.6, 31.4, 31.2, 14.8. MS (ES+) found 247.15; C₁₅H₂₂N₂O (M⁺+H) requires 247.18.

1-(3-(t-butyl)phenyl)-3-cyclopentylurea (24). Flash chromatography (hexanes/ethyl acetate (90:10)) Yield: 57%; White solid; mp: 152-154° C.; ¹H NMR (CDCl₃ and MeOD): δ 7.33-7.31 (m, 1H, Ar), 7.16-7.13 (m, 1H, Ar), 7.09-7.01 (m, 2H, Ar), 4.05 (quint, 1H, J=6.8 Hz, CH), 1.96-1.85 (m, 2H, 2× CH), 1.63-1.48 (m, 4H, 2× CH₂), 1.38-1.29 (m, 2H, 2× CH), 1.25 (s, 9H, 3× CH₃); ¹³C NMR (CDCl₃ and MeOD): δ 156.3, 152.3, 138.6, 128.6, 120.2, 117.4, 117.3, 51.7, 34.6, 33.3, 31.2, 23.5. MS (ES+) found 261.25; C₁₆H₂₄N₂O (M⁺+H) requires 261.20.

1-(3-(t-Butyl)phenyl)-3-(cycloheptylmethyl)urea (25). Flash chromatography (hexanes/ethyl acetate (90:10)) Yield: 53%; White solid; mp: 113-114° C.; ¹H NMR (CDCl₃ and MeOD): δ 7.33-7.31 (m, 1H, Ar), 7.21-7.16 (m, 1H, Ar), 7.10-7.04 (m, 2H, Ar), 3.12 (d, 2H, J=7.3 Hz, CH₂), 1.98 (sept, 1H, J=7.7 Hz, CH), 1.74-1.64 (m, 2H, 2× CH), 1.62-1.45 (m, 4H, 2× CH₂), 1.26 (s, 9H, 3× CH₃), 1.21-1.09 (m, 2H, 2× CH); ¹³C NMR (CDCl₃ and MeOD): δ 156.7, 152.4, 138.5, 128.7, 120.6, 118.0, 117.9, 45.0, 40.0, 34.7, 31.2, 30.2, 25.2. MS (ES+) found 275.20; C₁₇H₂₆N₂O (M⁺+H) requires 275.21.

1-(3-(t-Butyl)phenyl)-3-(cyclopent-3-en-1-yl)urea (26). Flash chromatography (hexanes/ethyl acetate (90:10)) Yield: 34%; White solid; mp: 135-137° C.; ¹H NMR (CDCl₃): δ 7.32-7.30 (m, 1H, Ar), 7.22-7.17 (m, 1H, Ar), 7.09-7.06 (m, 2H, Ar), 6.94 (s, 1H, NH)—, 5.66 (s, 2H, 2× CH), 5.44 (d, 1H, J=7.4 Hz, NH), 4.52-4.41 (m, 1H, CH), 2.73 (dd, 2H, J=7.6 Hz, 2× CH), 2.17 (dd, 2H, J=3.8 Hz, 2× CH), 1.27 (s, 9H, 3× CH₃); ¹³C NMR (CDCl₃): δ 156.0, 152.5, 138.5, 128.9, 128.9, 120.7, 118.1, 118.0, 49.8, 40.5, 34.7, 31.3. MS (ES+) found 259.10; C₁₆H₂₂N₂O (M⁺+H) requires 259.18.

1-(3-(t-Butyl)phenyl)-3-neopentylurea (27). Flash chromatography (hexanes/ethyl acetate (90:10)) Yield: 26%; White solid; mp: 145-147° C.; ¹H NMR (CDCl₃): δ 7.34-7.31 (m, 1H, Ar), 7.24-7.19 (m, 1H, Ar), 7.12-7.08 (m, 2H, Ar), 7.02 (s, 1H, NH), 5.30 (s, 1H, NH), 3.03 (s, 2H, CH₂), 1.28 (s, 9H, 3× CH₃), 0.88 (s, 9H, 3× CH₃); ¹³C NMR (CDCl₃): δ 156.7, 152.6, 138.5, 128.9, 120.9, 118.6, 118.4, 51.4, 34.7, 32.0, 31.3, 27.2. MS (ES+) found 263.20; C₁₆H₂₆N₂O (M⁺+H) requires 263.21.

1-(3-(t-Butyl)phenyl)-3-(2-methoxyethyl)urea (28). Flash chromatography (hexanes/ethyl acetate (80:20)) Yield: 61%; White solid; mp: 119-120° C.; ¹H NMR (CDCl₃ and MeOD): δ 7.33-7.32 (m, 1H, Ar), 7.21-7.16 (m, 1H, Ar), 7.11-7.03 (m, 2H, Ar), 3.47 (t, 2H, J=5.0 Hz, CH₂), 3.39 (t, 2H, J=5.0 Hz, CH₂), 3.33 (s, 3H, CH₃), 1.27 (s, 9H, 3× CH₃); ¹³C NMR (CDCl₃ and MeOD): δ 156.7, 152.3, 138.5, 128.7, 120.4, 117.5, 117.5, 72.1, 58.7, 39.9, 34.7, 31.2. MS (ES+) found 251.15; C₁₄H₂₂N₂O₂ (M⁺+H) requires 251.18.

1-(3-(t-Butyl)phenyl)-3-(pentan-3-yl)urea (29). Flash chromatography (hexanes/ethyl acetate (80:20)) Yield: 11%; White solid; mp: 159-161° C.; ¹H NMR (CDCl₃): δ 7.35-7.34 (m, 1H, Ar), 7.31-7.29 (m, 1H, Ar), 7.19-7.12 (m, 2H, Ar), 6.54 (s, 1H, NH), 4.65 (s, 1H, NH), 3.73 (quint, 1H, J=5.8 Hz, CH), 1.64-1.52 (m, 2H, 2× CH), 1.48-1.36 (m, 2H, 2× CH), 1.34 (s, 9H, 3× CH₃), 0.95 (t, 6H, J=7.4 Hz, 2× CH₃); ¹³C NMR (CDCl₃): δ 156.0, 152.8, 138.3, 129.1, 121.3, 119.0, 118.9, 52.9, 34.8, 31.3, 27.7, 10.2. MS (ES+) found 263.20; C₁₆H₂₆N₂O (M⁺+H) requires 263.21.

1-(4-Cyclohexylphenyl)-3-(cyclopropylmethyl)urea (30). Flash chromatography (hexanes/ethyl acetate (80:20)) Yield: 72%; White solid; mp: 178-181° C.; ¹H NMR (CDCl₃ and MeOD): δ 7.12 (d, 2H, J=8.6 Hz, Ar), 7.00 (d, 2H, J=8.4 Hz, Ar), 2.96 (d, 2H, J=7.0 Hz, CH₂), 2.39-2.29 (m, 1H, CH), 1.74-1.12 (m, 10H, 5× CH₂), 0.90-0.81 (m, 1H, CH), 0.41-0.34 (m, 2H, 2× CH), 0.11-0.05 (m, 2H, 2× CH); ¹³C NMR (CDCl₃ and MeOD): δ 156.8, 142.7, 136.6, 127.1, 119.9, 44.5, 43.8, 34.4, 26.8, 26.0, 10.9, 2.9. MS (ES+) found 273.15; C₁₇H₂₄N₂O (M⁺+H) requires 273.20.

1-Cyclobutyl-3-(4-cyclohexylphenyl)urea (31). Flash chromatography (hexanes/ethyl acetate (80:20)) Yield: 11%; White solid; mp: 137-139° C.; ¹H NMR (CDCl₃ and MeOD): δ 7.16 (d, 2H, J=8.6 Hz, Ar), 7.10 (d, 2H, J=8.5 Hz, Ar), 6.85 (s, 1H, NH), 4.22 (quint, 1H, J=7.7 Hz, CH), 2.34-2.27 (m, 2H, CH₂), 1.81-1.22 (m, 15H, CH+7× CH₂); ¹³C NMR (CDCl₃ and MeOD): δ 155.7, 146.1, 136.1, 127.5, 121.2, 45.3, 43.9, 34.5, 31.5, 26.9, 26.1, 14.8. MS (ES+) found 273.20; C₁₇H₂₄N₂O (M⁺+H) requires 273.20.

1-(4-Cyclohexylphenyl)-3-(cycloheptylmethyl)urea (32). Flash chromatography (hexanes/ethyl acetate (80:20)) Yield: 14%; White solid; mp: 142-144° C.; ¹H NMR (CDCl₃ and MeOD): δ 7.16 (apparent s, 4H, Ar), 6.37 (s, 1H, NH), 3.17 (d, 2H, J=7.3 Hz, CH₂), 2.51-2.41 (m, 1H, CH), 2.03 (sept, 1H, J=7.7 Hz, CH), 1.85-1.25 (m, 18H, 9× CH₂); ¹³C NMR (CDCl₃ and MeOD): δ 156.3, 144.6, 135.9, 127.8, 122.4, 45.4, 44.0, 40.1, 34.5, 30.3, 26.9, 26.2, 25.3. MS (ES+) found 301.20; C₁₉H₂₈N₂O (M⁺+H) requires 301.23.

1-(4-Cyclohexylphenyl)-3-(cyclopent-3-en-1-yl)urea (33). Flash chromatography (hexanes/ethyl acetate (80:20)) Yield: 41%; White solid; mp: 167-169° C.; ¹H NMR (CDCl₃ and MeOD): δ 7.15 (d, 2H, J=8.4 Hz, Ar), 7.05 (d, 2H, J=8.4 Hz, Ar), 5.63 (apparent s, 2H, 2× CH), 4.41-4.32 (m, 1H, CH), 2.68 (dd, 2H, J=7.6 Hz, 2× CH), 2.43-2.35 (m, 1H, CH), 2.16-2.09 (m, 2H, 2× CH), 1.83-1.15 (m, 10H, 5× CH₂); ¹³C NMR (CDCl₃ and MeOD): δ 156.4, 143.1, 136.5, 128.9, 127.3, 120.3, 49.5, 43.9, 40.3, 34.5, 26.9, 26.1. MS (ES+) found 285.20; C₁₈H₂₄N₂O (M⁺+H) requires 285.20.

1-(4-Cyclohexylphenyl)-3-neopentylurea (34). Flash chromatography (hexanes/ethyl acetate (80:20)) Yield: 21%; White solid; mp: 167-169° C.; ¹H NMR (CDCl₃ and MeOD): δ 7.18 (d, 2H, J=8.4 Hz, Ar), 7.08 (d, 2H, J=8.5 Hz, Ar), 2.96 (s, 2H, CH₂), 2.44-2.36 (m, 1H, CH), 1.80-1.16 (m, 10H, 5× CH₂), 0.85 (s, 9H, 3× CH₃); ¹³C NMR (CDCl₃ and MeOD): δ 156.9, 143.3, 136.5, 127.4, 120.7, 51.1, 43.9, 34.5, 32.0, 27.1, 26.9, 26.1. MS (ES+) found 289.20; C₁₈H₂₈N₂O (M⁺+H) requires 289.23.

1-(4-Cyclohexylphenyl)-3-(pentan-3-yl)urea (35). Flash chromatography (hexanes/ethyl acetate (80:20)) Yield: 14%; White solid; mp: 174-176° C.; ¹H NMR (CDCl₃ and MeOD): δ 7.17 (d, 2H, J=8.4 Hz, Ar), 7.10 (d, 2H, J=8.5 Hz, Ar), 3.59 (quint, 1H, J=5.6 Hz, CH), 2.46-2.36 (m, 1H, CH), 1.86-1.22 (m, 14H, 7× CH₂), 0.87 (t, 6H, 2× CH₃); ¹³C NMR (CDCl₃ and MeOD): δ 156.5, 143.6, 136.4, 127.5, 121.1, 52.6, 43.9, 34.5, 27.7, 26.9, 26.1, 10.2. MS (ES+) found 289.15; C₁₈H₂₈N₂O (M⁺+H) requires 289.23.

1-(4-Iodophenyl)-3-(pentan-3-yl)urea (36). Flash chromatography (hexanes/ethyl acetate (80:20)) Yield: 8%; White solid; mp: 174-176° C.; ¹H NMR (CDCl₃ and MeOD): δ 7.49 (d, 2H, J=8.7 Hz, Ar), 7.10 (d, 2H, J=8.8 Hz, Ar), 3.60-3.52 (m, 1H, CH), 1.55-1.40 (m, 2H, 2× CH), 1.34-1.23 (m, 2H, 2× CH), 0.86 (t, 6H, J=7.4 Hz, 2× CH₃); ¹³C NMR (CDCl₃ and MeOD): δ 155.9, 139.5, 137.7, 120.9, 84.7, 52.4, 27.6, 10.1. MS (ES+) found 333.00; C₁₂H₁₇IN₂O (M⁺+H) requires 333.05.

1-(4-(t-Butyl)phenyl)-3-neopentylurea (37). Flash chromatography (hexanes/ethyl acetate (80:20)) Yield: 52%; White solid; mp: 185-187° C.; ¹H NMR (CDCl₃ and MeOD): δ 7.74 (s, 1H, NH), 7.25-7.15 (apparent s, 4H, Ar), 5.77 (s, 1H, NH), 2.95 (s, 2H, CH₂), 1.23 (s, 9H, 3× CH₃), 0.83 (s, 9H, 3× CH₃); ¹³C NMR (CDCl₃ and MeOD): δ 157.1, 145.5, 136.6, 125.7, 119.7, 119.5, 51.1, 34.2, 32.0, 31.4, 27.1. MS (ES+) found 263.20; C₁₆H₂₆N₂O (M⁺+H) requires 263.21.

1-(4-(t-Butyl)phenyl)-3-(cyclopent-3-en-1-yl)urea (38). Flash chromatography (hexanes/ethyl acetate (80:20)) Yield: 34%; White solid; mp: 179-184° C.; ¹H NMR (CDCl₃ and DMSO-d₆): δ), 7.75 (s, 1H, NH), 7.14-7.02 (m, 4H, Ar), 3.93-3.85 (m, 1H, CH), 1.80-1.70 (m, 2H, CH₂), 1.53-1.34 (m, 4H, 2× CH₂), 1.25-1.15 (m, 2H, CH₂), 1.11-1.07 (m, 9H, 3× CH₃); ¹³C NMR (CDCl₃ and DMSO-d₆): δ 155.8, 144.2, 137.4, 125.4, 118.2, 51.3, 33.9, 33.3, 31.3, 23.4. MS (ES+) found 259.20; C₁₆H₂₄N₂O (M⁺+H) requires 259.18.

While the present disclosure has been described with reference to specific examples, it is to be understood that the disclosure is not limited to the disclosed examples. To the contrary, the disclosure is intended to cover various modifications and equivalent arrangements included within the spirit and scope of the appended claims.

All publications, patents and patent applications cited in the present disclosure are herein incorporated by reference in their entirety to the same extent as if each individual publication, patent or patent application was specifically and individually indicated to be incorporated by reference in its entirety.

### REFERENCES

1. J. S. Fortin, J. Lacroix, M. Desjardins, A. Patenaude, E. Petitclerc, R. C.-Gaudreault, Alkylation potency and protein specificity of aromatic urea derivatives and bioisosteres as potential irreversible antagonists of the colchicine-binding site, Bioorg Med Chem, 15 (2007) 4456-4469.
2. E. Mounetou, J. Legault, J. Lacroix, C. G. R, Antimitotic antitumor agents: synthesis, structure-activity relationships, and biological characterization of N-aryl-N′-(2-chloroethyl)ureas as new selective alkylating agents, Journal of medicinal chemistry, 44 (2001) 694-702.
3. J. S. Fortin, M. F. Cote, J. Lacroix, A. Patenaude, E. Petitclerc, R. C.-Gaudreault, Cycloalkyl-substituted aryl chloroethylureas inhibiting cell cycle progression in G0/G1 phase and thioredoxin-1 nuclear translocation, Bioorg Med Chem Lett, 18 (2008) 3526-3531.
4. J. Lacroix, R. C.-Gaudreault, M. Page, L. P. Joly, In vitro and in vivo activity of 1-aryl-3-(2-chloroethyl) urea derivatives as new antineoplastic agents, Anticancer Res, 8 (1988) 595-598.
5. P. Béchard, J. Lacroix, J. Poyet, R. C.-Gaudreault, Synthesis and cytotoxic activity of new alkyl-[3-(chloroethyl)ureido] benzene derivatives, Eur J Med Chem, 29 (1994) 963-966.
6. R. C.-Gaudreault, M. A. Alaoui-Jamali, G. Batist, P. Bechard, J. Lacroix, P. Poyet, Lack of cross-resistance to a new cytotoxic arylchloroethyl urea in various drug-resistant tumor cells, Cancer Chemother Pharmacol, 33 (1994) 489-492.
7. A. Patenaude, R. G. Deschesnes, J. L. Rousseau, E. Petitclerc, J. Lacroix, M. F. Cote, R. C.-Gaudreault, New soft alkylating agents with enhanced cytotoxicity against cancer cells resistant to chemotherapeutics and hypoxia, Cancer Res, 67 (2007) 2306-2316.
8. S. Fortin, B. Bouchon, C. Chambon, J. Lacroix, E. Moreau, J. M. Chezal, F. Degoul, C. G. R, Characterization of the covalent binding of N-phenyl-N′-(2-chloroethyl)ureas to {beta}-tubulin: importance of Glu198 in microtubule stability, The Journal of pharmacology and experimental therapeutics, 336 (2011) 460-467.
9. R. G. Deschesnes, A. Patenaude, J. L. Rousseau, J. S. Fortin, C. Ricard, M. F. Cote, J. Huot, R. C.-Gaudreault, E. Petitclerc, Microtubule-destabilizing agents induce focal adhesion structure disorganization and anoikis in cancer cells, J Pharmacol Exp Ther, 320 (2007) 853-864.
10. J. Fortin, A. Patenaude, R. G. Deschesnes, M. F. Cote, E. Petitclerc, C. G. R, ASK1-P38 pathway is important for anoikis induced by microtubule-targeting aryl chloroethylureas, J Pharm Pharm Sci, 13 (2010) 175-190.
11. T. Kishimoto, IL-6: from its discovery to clinical applications, International Immunology, 22 (2010) 347-352
12. C. A. Hunter, S. A. Jones, IL-6 as a keystone cytokine in health and disease, Nat Immunol, 16 (2015) 448-457
13. G. W. Kim, N. R. Lee, R. H. Pi, Y. S. Lim, Y. M. Lee, J. M. Lee, H. S. Jeong, S. H. Chung, IL-6 inhibitors for treatment of rheumatoid arthritis: past, present, and future, Arch Pharm Res, 38 (2015) 575-584.
14. A. Saggini, S. Chimenti, A. Chiricozzi, IL-6 as a druggable target in psoriasis: focus on pustular variants, J Immunol Res, 2014 (2014) 964069.
15. K. Taniguchi, M. Karin, IL-6 and related cytokines as the critical lynchpins between inflammation and cancer, Semin Immunol, 26 (2014) 54-74.
16. M. J. Waldner, M. F. Neurath, Master regulator of intestinal disease: IL-6 in chronic inflammation and cancer development, Seminars in Immunology, 26 (2014) 75-79.
17. H. Liu, R. Xu, L. Feng, W. Guo, N. Cao, C. Qian, P. Teng, L. Wang, X. Wu, Y. Sun, J. Li, Y. Shen, Q. Xu, A novel chromone derivative with anti-inflammatory property via inhibition of ROS-dependent activation of TRAF6-ASK1-p38 pathway, PLoS One, 7 (2012) e37168.
18. Raychaudhuri S. P., Farber E. M., (2001) The prevalence of psoriasis in the world. J Eur. Acad. Dermatol. Venereol 15: 16-17.
19. Griffiths C. E., Barker J. N. (2007) Pathogenesis and clinical features of psoriasis. Lancet 370: 263-271.
20. Ryan S (2010) Psoriasis: characteristics, psychosocial effects and treatment options. Br J Nurs 19: 820, 822-825.
21. Nestle F O, Kaplan D H, Barker J (2009) Psoriasis. The new England Journal of Medicine 361: 496-509.
22. McKay I A, Leigh I M (1995) Altered Keratinocyte Growth and Differentiation in Psoriasis. Clinics in Dermatology 13: 105-114.
23. Bernard B A, Asselineau D, Schaffar-Deshayes L, Darmon M Y (1988) Abnormal sequence of expression of differentiation markers in psoriatic epidermis: inversion of two steps in the differentiation program? J Invest Dermatol 90: 801-805.
24. Giardina E, Capon F, De Rosa M C, Mango R, Zambruno G, et al. (2004) Characterization of the loricrin (LOR) gene as a positional candidate for the PSORS4 psoriasis susceptibility locus. Ann Hum Genet 68: 639-645.
25. Heidenreich R, Rocken M, Ghoreschi K (2009) Angiogenesis drives psoriasis pathogenesis. Int J Exp Pathol 90: 232-248.
26. Gaspari A A (2006) Innate and adaptive immunity and the pathophysiology of psoriasis. J Am Acad. Dermatol 54: S67-80.
27. V. P. Menon, A. R. Sudheer, Antioxidant and anti-inflammatory properties of curcumin, Adv Exp Med Biol, 595 (2007) 105-125.
28. K. D. Rainsford, Ibuprofen: pharmacology, efficacy and safety, Inflammopharmacology, 17 (2009) 275-342.
29. R. H. Shoemaker, The NCI60 human tumour cell line anticancer drug screen, Nature reviews. Cancer, 6 (2006) 813-823.
30. S. Fortin, L. Wei, E. Moreau, J. Lacroix, M. F. Cote, E. Petitclerc, L. P. Kotra, C. G. R, Design, synthesis, biological evaluation, and structure-activity relationships of substituted phenyl 4-(2-oxoimidazolidin-1-yl)benzenesulfonates as new tubulin inhibitors mimicking combretastatin A-4, Journal of medicinal chemistry, 54 (2011) 4559-4580.
31. M. J. Cecchini, M. Amiri, F. A. Dick, Analysis of Cell Cycle Position in Mammalian Cells, Journal of Visualized Experiments: JoVE, (2012) 3491.
32. C. Riccardi, I. Nicoletti, Analysis of apoptosis by propidium iodide staining and flow cytometry, Nat. Protocols, 1 (2006) 1458-1461.
33. J. Legault, J. F. Gaulin, E. Mounetou, S. Bolduc, J. Lacroix, P. Poyet, R. C.-Gaudreault, Microtubule disruption induced in vivo by alkylation of beta-tubulin by 1-aryl-3-(2-chloroethyl)ureas, a novel class of soft alkylating agents, Cancer Res, 60 (2000) 985-992.
34. T. Nagasaki, M. Hara, H. Nakanishi, H. Takahashi, M. Sato, H. Takeyama, Interleukin-6 released by colon cancer-associated fibroblasts is critical for tumour angiogenesis: anti-interleukin-6 receptor antibody suppressed angiogenesis and inhibited tumour-stroma interaction, British Journal of Cancer, 110 (2014) 469-478.
35. L. Sirota, D. Shacham, I. Punsky, H. Bessler, Ibuprofen affects pro- and anti-inflammatory cytokine production by mononuclear cells of preterm newborns, Biology of the neonate, 79 (2001) 103-108.
36. L. Gallelli, O. Galasso, D. Falcone, S. Southworth, M. Greco, V. Ventura, P. Romualdi, A. Corigliano, R. Terracciano, R. Savino, E. Gulletta, G. Gasparini, G. De Sarro, The effects of nonsteroidal anti-inflammatory drugs on clinical outcomes, synovial fluid cytokine concentration and signal transduction pathways in knee osteoarthritis. A randomized open label trial, Osteoarthritis and Cartilage, 21 (2013) 1400-1408.
37. P. Wojdasiewicz, L. A. Poniatowski, D. Szukiewicz, The role of inflammatory and anti-inflammatory cytokines in the pathogenesis of osteoarthritis, Mediators Inflamm, 2014 (2014) 561459.
38. J. Fortin, m. o. Benoit-Biancamano, Inhibition of Islet Amyloid PolypeptideAggregation and Associated Cytotoxicity by Nonsteroidal Anti-Inflammatory Drugs, Canadian Journal of Physiology and Pharmacology, 94 (2015) 35-48.
39. Y. Zhou, Y. Su, B. Li, F. Liu, J. W. Ryder, X. Wu, P. A. Gonzalez-DeWhitt, V. Gelfanova, J. E. Hale, P. C. May, S. M. Paul, B. Ni, Nonsteroidal anti-inflammatory drugs can lower amyloidogenic Abeta42 by inhibiting Rho, Science, 302 (2003) 1215-1217.
40. H. Akrami, S. Aminzadeh, H. Fallahi, Inhibitory effect of ibuprofen on tumor survival and angiogenesis in gastric cancer cell, Tumour Biol, 36 (2015) 3237-3243.

1. A compound of Formula I: 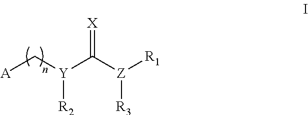
wherein:
A is an arene or a heteroarene;
Y is N, O or S;
Z is N, O or S;
X is O, S or N═CN;
R₁ is a (C₄₋₁₅)-branched alkyl; (C₃₋₈)cycloalkyl; (C₃₋₈)cycloalkyl having at least one substituent selected from (C₁₋₁₀)alkyl, (C₄₋₁₀)branched alkyl, —O—(C₁-C₁₀)alkyl, —S—(C₁-C₁₀)alkyl, —NRR, —NO₂, —CN, —C(O)R, —C(S)R, —C(O)OR, —C(S)OR, —SC(S)R, —OC(S)R, —C(O)NRR, —C(S)NRR, —C(O)NR(OR), —C(S)NR(OR), —C(O)NR(SR), —C(S)NR(SR), —CH(CN)₂, —CH[C(O)R]₂, —CH[C(S)R]₂, —CH[C(O)OR]₂, —CH[C(S)OR]₂, —CH[C(O)SR]₂, —CH[C(S)SR]₂, —NRC(O)R, —NRC(O)OR, —S(O)—R, —S(O)OR, and —S(O)NRR; (C₆₋₁₈)aryl; or (C₆₋₁₈)aryl having at least one substituent selected from (C₁₋₁₀)alkyl, (C₄₋₁₀)branched alkyl, —O—(C₁-C₁₀)alkyl, —S—(C₁-C₁₀)alkyl, —NRR, —NO₂, —CN, —C(O)R, —C(S)R, —C(O)OR, —C(S)OR, —SC(S)R, —OC(S)R, —C(O)NRR, —C(S)NRR, —C(O)NR(OR), —C(S)NR(OR), —C(O)NR(SR), —C(S)NR(SR), —CH(CN)₂, —CH[C(O)R]₂, —CH[C(S)R]₂, —CH[C(O)OR]₂, —CH[C(S)OR]₂, —CH[C(O)SR]₂, —CH[C(S)SR]₂, —NRC(O)R, —NRC(O)OR, —S(O)—R, —S(O)OR, and —S(O)NRR;
R₂ and R₃ are independently hydrogen, (C₁₋₁₀)alkyl, (C₄₋₁₀)branched alkyl, (C₃₋₈)cycloalkyl; (C₃₋₈)cycloalkyl having at least one substituent selected from (C₁₋₁₀)alkyl, (C₄₋₁₀)branched alkyl, —O—(C₁-C₁₀)alkyl, —S—(C₁-C₁₀)alkyl, —NRR, —NO₂, —CN, —C(O)R, —C(S)R, —C(O)OR, —C(S)OR, —SC(S)R, —OC(S)R, —C(O)NRR, —C(S)NRR, —C(O)NR(OR), —C(S)NR(OR), —C(O)NR(SR), —C(S)NR(SR), —CH(CN)₂, —CH[C(O)R]₂, —CH[C(S)R]₂, —CH[C(O)OR]₂, —CH[C(S)OR]₂, —CH[C(O)SR]₂, —CH[C(S)SR]₂, —NRC(O)R, —NRC(O)OR, —S(O)—R, —S(O)OR, and —S(O)NRR; (C₆₋₁₈)aryl; or (C₆₋₁₈)aryl having at least one substituent selected from (C₁₋₁₀)alkyl, (C₄₋₁₀)branched alkyl, —O—(C₁-C₁₀)alkyl, —S—(C₁-C₁₀)alkyl, —NRR, —NO₂, —CN, —C(O)R, —C(S)R, —C(O)OR, —C(S)OR, —SC(S)R, —OC(S)R, —C(O)NRR, —C(S)NRR, —C(O)NR(OR), —C(S)NR(OR), —C(O)NR(SR), —C(S)NR(SR), —CH(CN)₂, —CH[C(O)R]₂, —CH[C(S)R]₂, —CH[C(O)OR]₂, —CH[C(S)OR]₂, —CH[C(O)SR]₂, —CH[C(S)SR]₂, —NRC(O)R, —NRC(O)OR, —S(O)—R, —S(O)OR, and —S(O)NRR; or
R₂ and R₃ are linked together to form a nitrogen containing ring system, wherein the nitrogen containing ring system is optionally substituted by at least one substituent selected from (C₁₋₁₀)alkyl, (C₄₋₁₀)branched alkyl, —O—(C₁-C₁₀)alkyl, —S—(C₁-C₁₀)alkyl, —NRR, —NO₂, —CN, —C(O)R, —C(S)R, —C(O)OR, —C(S)OR, —SC(S)R, —OC(S)R, —C(O)NRR, —C(S)NRR, —C(O)NR(OR), —C(S)NR(OR), —C(O)NR(SR), —C(S)NR(SR), —CH(CN)₂, —CH[C(O)R]₂, —CH[C(S)R]₂, —CH[C(O)OR]₂, —CH[C(S)OR]₂, —CH[C(O)SR]₂, —CH[C(S)SR]₂, —NRC(O)R, —NRC(O)OR, —S(O)—R, —S(O)OR, and —S(O)NRR;
n is 0, 1 or 2;
wherein:
A is optionally substituted with one or more substituents selected from the group of (C₁-C₁₅)alkyl, (C₂-C₁₅)alkenyl, (C₂-C₁₅)alkynyl, —O—(C₁-C₁₅)alkyl, —O—(C₂-C₁₅) alkenyl, —O—(C₂-C₁₅) alkynyl, —S—(C₁-C₁₅)alkyl, —S—(C₂-C₁₅)alkenyl, —S—(C₂-C₁₅)alkynyl, (C₃-C₈)cycloalkyl, —O—(C₃-C₈)cycloalkyl, —S—(C₃-C₈)cycloalkyl, -halo, —NRR, —NO₂, —CN, —C(O)R, —C(S)R, —C(O)OR, —C(S)OR, —SC(S)R, —OC(S)R, —C(O)NRR, —C(S)NRR, —C(O)NR(OR), —C(S)NR(OR), —C(O)NR(SR), —C(S)NR(SR), —CH(CN)₂, —CH[C(O)R]₂, —CH[C(S)R]₂, —CH[C(O)OR]₂, —CH[C(S)OR]₂, —CH[C(O)SR]₂, —CH[C(S)SR]₂, —NRC(O)R, —NRC(O)OR, —S(O)—R, —S(O)OR, and —S(O)NRR;
wherein
each R is independently selected from —H, (C₁-C₁₀)alkyl, (C₂-C₁₀)alkenyl, (C₂-C₁₀)alkynyl, (C₃-C₈)cycloalkyl, aryl or substituted aryl;
wherein
when Y and/or Z are O or S, R₂ and/or R₃ are absent;
or a pharmaceutically acceptable salt, a prodrug, a tautomer or a solvate thereof.

2. A compound of Formula I: 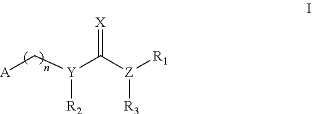
wherein:
A is an arene or a heteroarene;
Y is N, O or S;
Z is N, O or S;
X is O, S or N═CN;
R₁ is a (C₄₋₁₅)-branched alkyl; (C₃₋₈)cycloalkyl; (C₃₋₈)cycloalkyl having at least one substituent selected from (C₁₋₁₀)alkyl, (C₄₋₁₀)branched alkyl, —O—(C₁-C₁₀)alkyl, —S—(C₁-C₁₀)alkyl, —NRR, —NO₂, —CN, —C(O)R, —C(S)R, —C(O)OR, —C(S)OR, —SC(S)R, —OC(S)R, —C(O)NRR, —C(S)NRR, —C(O)NR(OR), —C(S)NR(OR), —C(O)NR(SR), —C(S)NR(SR), —CH(CN)₂, —CH[C(O)R]₂, —CH[C(S)R]₂, —CH[C(O)OR]₂, —CH[C(S)OR]₂, —CH[C(O)SR]₂, —CH[C(S)SR]₂, —NRC(O)R, —NRC(O)OR, —S(O)—R, —S(O)OR, and —S(O)NRR; (C₆₋₁₈)aryl; or (C₆₋₁₈)aryl having at least one substituent selected from (C₁₋₁₀)alkyl, (C₄₋₁₀)branched alkyl, —O—(C₁-C₁₀)alkyl, —S—(C₁-C₁₀)alkyl, —NRR, —NO₂, —CN, —C(O)R, —C(S)R, —C(O)OR, —C(S)OR, —SC(S)R, —OC(S)R, —C(O)NRR, —C(S)NRR, —C(O)NR(OR), —C(S)NR(OR), —C(O)NR(SR), —C(S)NR(SR), —CH(CN)₂, —CH[C(O)R]₂, —CH[C(S)R]₂, —CH[C(O)OR]₂, —CH[C(S)OR]₂, —CH[C(O)SR]₂, —CH[C(S)SR]₂, —NRC(O)R, —NRC(O)OR, —S(O)—R, —S(O)OR, and —S(O)NRR;
R₂ and R₃ are independently hydrogen, (C₁₋₁₀)alkyl, (C₄₋₁₀)branched alkyl, (C₃₋₈)cycloalkyl; (C₃₋₈)cycloalkyl having at least one substituent selected from (C₁₋₁₀)alkyl, (C₄₋₁₀)branched alkyl, —O—(C₁-C₁₀)alkyl, —S—(C₁-C₁₀)alkyl, —NRR, —NO₂, —CN, —C(O)R, —C(S)R, —C(O)OR, —C(S)OR, —SC(S)R, —OC(S)R, —C(O)NRR, —C(S)NRR, —C(O)NR(OR), —C(S)NR(OR), —C(O)NR(SR), —C(S)NR(SR), —CH(CN)₂, —CH[C(O)R]₂, —CH[C(S)R]₂, —CH[C(O)OR]₂, —CH[C(S)OR]₂, —CH[C(O)SR]₂, —CH[C(S)SR]₂, —NRC(O)R, —NRC(O)OR, —S(O)—R, —S(O)OR, and —S(O)NRR; (C₆₋₁₈)aryl; or (C₆₋₁₈)aryl having at least one substituent selected from (C₁₋₁₀)alkyl, (C₄₋₁₀)branched alkyl, —O—(C₁-C₁₀)alkyl, —S—(C₁-C₁₀)alkyl, —NRR, —NO₂, —CN, —C(O)R, —C(S)R, —C(O)OR, —C(S)OR, —SC(S)R, —OC(S)R, —C(O)NRR, —C(S)NRR, —C(O)NR(OR), —C(S)NR(OR), —C(O)NR(SR), —C(S)NR(SR), —CH(CN)₂, —CH[C(O)R]₂, —CH[C(S)R]₂, —CH[C(O)OR]₂, —CH[C(S)OR]₂, —CH[C(O)SR]₂, —CH[C(S)SR]₂, —NRC(O)R, —NRC(O)OR, —S(O)—R, —S(O)OR, and —S(O)NRR; or
R₂ and R₃ are linked together to form a nitrogen containing ring system, wherein the nitrogen containing ring system is optionally substituted by at least one substituent selected from (C₁₋₁₀)alkyl, (C₄₋₁₀)branched alkyl, —O—(C₁-C₁₀)alkyl, —S—(C₁-C₁₀)alkyl, —NRR, —NO₂, —CN, —C(O)R, —C(S)R, —C(O)OR, —C(S)OR, —SC(S)R, —OC(S)R, —C(O)NRR, —C(S)NRR, —C(O)NR(OR), —C(S)NR(OR), —C(O)NR(SR), —C(S)NR(SR), —CH(CN)₂, —CH[C(O)R]₂, —CH[C(S)R]₂, —CH[C(O)OR]₂, —CH[C(S)OR]₂, —CH[C(O)SR]₂, —CH[C(S)SR]₂, —NRC(O)R, —NRC(O)OR, —S(O)—R, —S(O)OR, and —S(O)NRR;
n is 0, 1 or 2;
wherein:
A is optionally substituted with one or more substituents selected from the group of (C₁-C₁₅)alkyl, (C₂-C₁₅)alkenyl, (C₂-C₁₅)alkynyl, —O—(C₁-C₁₅)alkyl, —O—(C₂-C₁₅) alkenyl, —O—(C₂-C₁₅) alkynyl, —S—(C₁-C₁₅)alkyl, —S—(C₂-C₁₅)alkenyl, —S—(C₂-C₁₅)alkynyl, (C₃-C₈)cycloalkyl, —O—(C₃-C₈)cycloalkyl, —S—(C₃-C₈)cycloalkyl, -halo, —NRR, —NO₂, —CN, —C(O)R, —C(S)R, —C(O)OR, —C(S)OR, —SC(S)R, —OC(S)R, —C(O)NRR, —C(S)NRR, —C(O)NR(OR), —C(S)NR(OR), —C(O)NR(SR), —C(S)NR(SR), —CH(CN)₂, —CH[C(O)R]₂, —CH[C(S)R]₂, —CH[C(O)OR]₂, —CH[C(S)OR]₂, —CH[C(O)SR]₂, —CH[C(S)SR]₂, —NRC(O)R, —NRC(O)OR, —S(O)—R, —S(O)OR, and —S(O)NRR;
wherein
each R is independently selected from —H, (C₁-C₁₀)alkyl, (C₂-C₁₀)alkenyl, (C₂-C₁₀)alkynyl, (C₃-C₈)cycloalkyl, aryl or substituted aryl;
wherein
when Y and/or Z are O or S, R₂ and/or R₃ are absent;
or a pharmaceutically acceptable salt, a prodrug, a tautomer or a solvate thereof;
for use as an anti-proliferative agent in the attenuation, inhibition, or prevention of conditions associated with IL-6 expression.

3. A compound of Formula I: 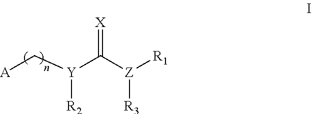
wherein:
A is an arene or a heteroarene;
Y is N, O or S;
Z is N, O or S;
X is O, S or N═CN;
R₁ is a (C₄₋₁₅)-branched alkyl; (C₃₋₈)cycloalkyl; (C₃₋₈)cycloalkyl having at least one substituent selected from (C₁₋₁₀)alkyl, (C₄₋₁₀)branched alkyl, —O—(C₁-C₁₀)alkyl, —S—(C₁-C₁₀)alkyl, —NRR, —NO₂, —CN, —C(O)R, —C(S)R, —C(O)OR, —C(S)OR, —SC(S)R, —OC(S)R, —C(O)NRR, —C(S)NRR, —C(O)NR(OR), —C(S)NR(OR), —C(O)NR(SR), —C(S)NR(SR), —CH(CN)₂, —CH[C(O)R]₂, —CH[C(S)R]₂, —CH[C(O)OR]₂, —CH[C(S)OR]₂, —CH[C(O)SR]₂, —CH[C(S)SR]₂, —NRC(O)R, —NRC(O)OR, —S(O)—R, —S(O)OR, and —S(O)NRR; (C₆₋₁₈)aryl; or
(C₆₋₁₈)aryl having at least one substituent selected from (C₁₋₁₀)alkyl, (C₄₋₁₀)branched alkyl, —O—(C₁-C₁₀)alkyl, —S—(C₁-C₁₀)alkyl, —NRR, —NO₂, —CN, —C(O)R, —C(S)R, —C(O)OR, —C(S)OR, —SC(S)R, —OC(S)R, —C(O)NRR, —C(S)NRR, —C(O)NR(OR), —C(S)NR(OR), —C(O)NR(SR), —C(S)NR(SR), —CH(CN)₂, —CH[C(O)R]₂, —CH[C(S)R]₂, —CH[C(O)OR]₂, —CH[C(S)OR]₂, —CH[C(O)SR]₂, —CH[C(S)SR]₂, —NRC(O)R, —NRC(O)OR, —S(O)—R, —S(O)OR, and —S(O)NRR;
R₂ and R₃ are independently hydrogen, (C₁₋₁₀)alkyl, (C₄₋₁₀)branched alkyl, (C₃₋₈)cycloalkyl; (C₃₋₈)cycloalkyl having at least one substituent selected from (C₁₋₁₀)alkyl, (C₄₋₁₀)branched alkyl, —O—(C₁-C₁₀)alkyl, —S—(C₁-C₁₀)alkyl, —NRR, —NO₂, —CN, —C(O)R, —C(S)R, —C(O)OR, —C(S)OR, —SC(S)R, —OC(S)R, —C(O)NRR, —C(S)NRR, —C(O)NR(OR), —C(S)NR(OR), —C(O)NR(SR), —C(S)NR(SR), —CH(CN)₂, —CH[C(O)R]₂, —CH[C(S)R]₂, —CH[C(O)OR]₂, —CH[C(S)OR]₂, —CH[C(O)SR]₂, —CH[C(S)SR]₂, —NRC(O)R, —NRC(O)OR, —S(O)—R, —S(O)OR, and —S(O)NRR; (C₆₋₁₈)aryl; or (C₆₋₁₈)aryl having at least one substituent selected from (C₁₋₁₀)alkyl, (C₄₋₁₀)branched alkyl, —O—(C₁-C₁₀)alkyl, —S—(C₁-C₁₀)alkyl, —NRR, —NO₂, —CN, —C(O)R, —C(S)R, —C(O)OR, —C(S)OR, —SC(S)R, —OC(S)R, —C(O)NRR, —C(S)NRR, —C(O)NR(OR), —C(S)NR(OR), —C(O)NR(SR), —C(S)NR(SR), —CH(CN)₂, —CH[C(O)R]₂, —CH[C(S)R]₂, —CH[C(O)OR]₂, —CH[C(S)OR]₂, —CH[C(O)SR]₂, —CH[C(S)SR]₂, —NRC(O)R, —NRC(O)OR, —S(O)—R, —S(O)OR, and —S(O)NRR; or
R₂ and R₃ are linked together to form a nitrogen containing ring system, wherein the nitrogen containing ring system is optionally substituted by at least one substituent selected from (C₁₋₁₀)alkyl, (C₄₋₁₀)branched alkyl, —O—(C₁-C₁₀)alkyl, —S—(C₁-C₁₀)alkyl, —NRR, —NO₂, —CN, —C(O)R, —C(S)R, —C(O)OR, —C(S)OR, —SC(S)R, —OC(S)R, —C(O)NRR, —C(S)NRR, —C(O)NR(OR), —C(S)NR(OR), —C(O)NR(SR), —C(S)NR(SR), —CH(CN)₂, —CH[C(O)R]₂, —CH[C(S)R]₂, —CH[C(O)OR]₂, —CH[C(S)OR]₂, —CH[C(O)SR]₂, —CH[C(S)SR]₂, —NRC(O)R, —NRC(O)OR, —S(O)—R, —S(O)OR, and —S(O)NRR;
n is 0, 1 or 2;
wherein:
A is optionally substituted with one or more substituents selected from the group of (C₁-C₁₅)alkyl, (C₂-C₁₅)alkenyl, (C₂-C₁₅)alkynyl, —O—(C₁-C₁₅)alkyl, —O—(C₂-C₁₅) alkenyl, —O—(C₂-C₁₅) alkynyl, —S—(C₁-C₁₅)alkyl, —S—(C₂-C₁₅)alkenyl, —S—(C₂-C₁₅)alkynyl, (C₃-C₈)cycloalkyl, —O—(C₃-C₈)cycloalkyl, —S—(C₃-C₈)cycloalkyl, -halo, —NRR, —NO₂, —CN, —C(O)R, —C(S)R, —C(O)OR, —C(S)OR, —SC(S)R, —OC(S)R, —C(O)NRR, —C(S)NRR, —C(O)NR(OR), —C(S)NR(OR), —C(O)NR(SR), —C(S)NR(SR), —CH(CN)₂, —CH[C(O)R]₂, —CH[C(S)R]₂, —CH[C(O)OR]₂, —CH[C(S)OR]₂, —CH[C(O)SR]₂, —CH[C(S)SR]₂, —NRC(O)R, —NRC(O)OR, —S(O)—R, —S(O)OR, and —S(O)NRR;
wherein
each R is independently selected from —H, (C₁-C₁₀)alkyl, (C₂-C₁₀)alkenyl, (C₂-C₁₀)alkynyl, (C₃-C₈)cycloalkyl, aryl or substituted aryl;
wherein
when Y and/or Z are O or S, R₂ and/or R₃ are absent;
or a pharmaceutically acceptable salt, a prodrug, a tautomer or a solvate thereof;
for use as a therapeutic agent in the attenuation, inhibition, or prevention of conditions associated with IL-6 expression.

4. A method for treating, attenuating, inhibiting, or preventing a condition associated with IL-6 expression in a subject in need thereof, the method comprising administering a therapeutically effective amount of a compound of Formula I: 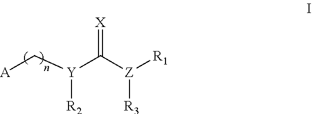
wherein:
A is an arene or a heteroarene;
Y is N, O or S;
Z is N, O or S;
X is O, S or N═CN;
R₁ is a (C₄₋₁₅)-branched alkyl; (C₃₋₈)cycloalkyl; (C₃₋₈)cycloalkyl having at least one substituent selected from (C₁₋₁₀)alkyl, (C₄₋₁₀)branched alkyl, —O—(C₁-C₁₀)alkyl, —S—(C₁-C₁₀)alkyl, —NRR, —NO₂, —CN, —C(O)R, —C(S)R, —C(O)OR, —C(S)OR, —SC(S)R, —OC(S)R, —C(O)NRR, —C(S)NRR, —C(O)NR(OR), —C(S)NR(OR), —C(O)NR(SR), —C(S)NR(SR), —CH(CN)₂, —CH[C(O)R]₂, —CH[C(S)R]₂, —CH[C(O)OR]₂, —CH[C(S)OR]₂, —CH[C(O)SR]₂, —CH[C(S)SR]₂, —NRC(O)R, —NRC(O)OR, —S(O)—R, —S(O)OR, and —S(O)NRR; (C₆₋₁₈)aryl; or (C₆₋₁₈)aryl having at least one substituent selected from (C₁₋₁₀)alkyl, (C₄₋₁₀)branched alkyl, —O—(C₁-C₁₀)alkyl, —S—(C₁-C₁₀)alkyl, —NRR, —NO₂, —CN, —C(O)R, —C(S)R, —C(O)OR, —C(S)OR, —SC(S)R, —OC(S)R, —C(O)NRR, —C(S)NRR, —C(O)NR(OR), —C(S)NR(OR), —C(O)NR(SR), —C(S)NR(SR), —CH(CN)₂, —CH[C(O)R]₂, —CH[C(S)R]₂, —CH[C(O)OR]₂, —CH[C(S)OR]₂, —CH[C(O)SR]₂, —CH[C(S)SR]₂, —NRC(O)R, —NRC(O)OR, —S(O)—R, —S(O)OR, and —S(O)NRR;
R₂ and R₃ are independently hydrogen, (C₁₋₁₀)alkyl, (C₄₋₁₀)branched alkyl, (C₃₋₈)cycloalkyl; (C₃₋₈)cycloalkyl having at least one substituent selected from (C₁₋₁₀)alkyl, (C₄₋₁₀)branched alkyl, —O—(C₁-C₁₀)alkyl, —S—(C₁-C₁₀)alkyl, —NRR, —NO₂, —CN, —C(O)R, —C(S)R, —C(O)OR, —C(S)OR, —SC(S)R, —OC(S)R, —C(O)NRR, —C(S)NRR, —C(O)NR(OR), —C(S)NR(OR), —C(O)NR(SR), —C(S)NR(SR), —CH(CN)₂, —CH[C(O)R]₂, —CH[C(S)R]₂, —CH[C(O)OR]₂, —CH[C(S)OR]₂, —CH[C(O)SR]₂, —CH[C(S)SR]₂, —NRC(O)R, —NRC(O)OR, —S(O)—R, —S(O)OR, and —S(O)NRR; (C₆₋₁₈)aryl; or (C₆₋₁₈)aryl having at least one substituent selected from (C₁₋₁₀)alkyl, (C₄₋₁₀)branched alkyl, —O—(C₁-C₁₀)alkyl, —S—(C₁-C₁₀)alkyl, —NRR, —NO₂, —CN, —C(O)R, —C(S)R, —C(O)OR, —C(S)OR, —SC(S)R, —OC(S)R, —C(O)NRR, —C(S)NRR, —C(O)NR(OR), —C(S)NR(OR), —C(O)NR(SR), —C(S)NR(SR), —CH(CN)₂, —CH[C(O)R]₂, —CH[C(S)R]₂, —CH[C(O)OR]₂, —CH[C(S)OR]₂, —CH[C(O)SR]₂, —CH[C(S)SR]₂, —NRC(O)R, —NRC(O)OR, —S(O)—R, —S(O)OR, and —S(O)NRR; or
R₂ and R₃ are linked together to form a nitrogen containing ring system, wherein the nitrogen containing ring system is optionally substituted by at least one substituent selected from (C₁₋₁₀)alkyl, (C₄₋₁₀)branched alkyl, —O—(C₁-C₁₀)alkyl, —S—(C₁-C₁₀)alkyl, —NRR, —NO₂, —CN, —C(O)R, —C(S)R, —C(O)OR, —C(S)OR, —SC(S)R, —OC(S)R, —C(O)NRR, —C(S)NRR, —C(O)NR(OR), —C(S)NR(OR), —C(O)NR(SR), —C(S)NR(SR), —CH(CN)₂, —CH[C(O)R]₂, —CH[C(S)R]₂, —CH[C(O)OR]₂, —CH[C(S)OR]₂, —CH[C(O)SR]₂, —CH[C(S)SR]₂, —NRC(O)R, —NRC(O)OR, —S(O)—R, —S(O)OR, and —S(O)NRR;
n is 0, 1 or 2;
wherein:
A is optionally substituted with one or more substituents selected from the group of (C₁-C₁₅)alkyl, (C₂-C₁₅)alkenyl, (C₂-C₁₅)alkynyl, —O—(C₁-C₁₅)alkyl, —O—(C₂-C₁₅) alkenyl, —O—(C₂-C₁₅) alkynyl, —S—(C₁-C₁₅)alkyl, —S—(C₂-C₁₅)alkenyl, —S—(C₂-C₁₅)alkynyl, (C₃-C₈)cycloalkyl, —O—(C₃-C₈)cycloalkyl, —S—(C₃-C₈)cycloalkyl, -halo, —NRR, —NO₂, —CN, —C(O)R, —C(S)R, —C(O)OR, —C(S)OR, —SC(S)R, —OC(S)R, —C(O)NRR, —C(S)NRR, —C(O)NR(OR), —C(S)NR(OR), —C(O)NR(SR), —C(S)NR(SR), —CH(CN)₂, —CH[C(O)R]₂, —CH[C(S)R]₂, —CH[C(O)OR]₂, —CH[C(S)OR]₂, —CH[C(O)SR]₂, —CH[C(S)SR]₂, —NRC(O)R, —NRC(O)OR, —S(O)—R, —S(O)OR, and —S(O)NRR;
wherein
each R is independently selected from —H, (C₁-C₁₀)alkyl, (C₂-C₁₀)alkenyl, (C₂-C₁₀)alkynyl, (C₃-C₈)cycloalkyl, aryl or substituted aryl;
wherein
when Y and/or Z are O or S, R₂ and/or R₃ are absent;
or a pharmaceutically acceptable salt, a prodrug, a tautomer or a solvate thereof.

5. A pharmaceutical composition comprising a compound as defined in any one of claims 1 to 3, and a pharmaceutically acceptable carrier.

6. A medicament for use in treating, attenuating, inhibiting and/or preventing inflammation and inflammation-related pathologies, the medicament comprising a compound as defined in any one of claims 1 to 3, and a pharmaceutically acceptable carrier.

7. A medicament for use in treating a condition associated with IL-6 expression, the medicament comprising a compound as defined in any one of claims 1 to 3, and a pharmaceutically acceptable carrier.

8. A compound of Formula II: 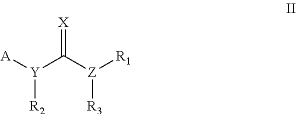
wherein:
A is an arene or a heteroarene;
Y is N, O or S;
Z is N, O or S;
X is O, S or N═CN;
R₁ is a (C₄₋₁₅)-branched alkyl; (C₃₋₈)cycloalkyl; (C₃₋₈)cycloalkyl having at least one substituent selected from (C₁₋₁₀)alkyl, (C₄₋₁₀)branched alkyl, —O—(C₁-C₁₀)alkyl, —S—(C₁-C₁₀)alkyl, —NRR, —NO₂, —CN, —C(O)R, —C(S)R, —C(O)OR, —C(S)OR, —SC(S)R, —OC(S)R, —C(O)NRR, —C(S)NRR, —C(O)NR(OR), —C(S)NR(OR), —C(O)NR(SR), —C(S)NR(SR), —CH(CN)₂, —CH[C(O)R]₂, —CH[C(S)R]₂, —CH[C(O)OR]₂, —CH[C(S)OR]₂, —CH[C(O)SR]₂, —CH[C(S)SR]₂, —NRC(O)R, —NRC(O)OR, —S(O)—R, —S(O)OR, and —S(O)NRR; (C₆₋₁₈)aryl; or (C₆₋₁₈)aryl having at least one substituent selected from (C₁₋₁₀)alkyl, (C₄₋₁₀)branched alkyl, —O—(C₁-C₁₀)alkyl, —S—(C₁-C₁₀)alkyl, —NRR, —NO₂, —CN, —C(O)R, —C(S)R, —C(O)OR, —C(S)OR, —SC(S)R, —OC(S)R, —C(O)NRR, —C(S)NRR, —C(O)NR(OR), —C(S)NR(OR), —C(O)NR(SR), —C(S)NR(SR), —CH(CN)₂, —CH[C(O)R]₂, —CH[C(S)R]₂, —CH[C(O)OR]₂, —CH[C(S)OR]₂, —CH[C(O)SR]₂, —CH[C(S)SR]₂, —NRC(O)R, —NRC(O)OR, —S(O)—R, —S(O)OR, and —S(O)NRR;
R₂ and R₃ are independently hydrogen, (C₁₋₁₀)alkyl, (C₄₋₁₀)branched alkyl, (C₃₋₈)cycloalkyl; (C₃₋₈)cycloalkyl having at least one substituent selected from (C₁₋₁₀)alkyl, (C₄₋₁₀)branched alkyl, —O—(C₁-C₁₀)alkyl, —S—(C₁-C₁₀)alkyl, —NRR, —NO₂, —CN, —C(O)R, —C(S)R, —C(O)OR, —C(S)OR, —SC(S)R, —OC(S)R, —C(O)NRR, —C(S)NRR, —C(O)NR(OR), —C(S)NR(OR), —C(O)NR(SR), —C(S)NR(SR), —CH(CN)₂, —CH[C(O)R]₂, —CH[C(S)R]₂, —CH[C(O)OR]₂, —CH[C(S)OR]₂, —CH[C(O)SR]₂, —CH[C(S)SR]₂, —NRC(O)R, —NRC(O)OR, —S(O)—R, —S(O)OR, and —S(O)NRR; (C₆₋₁₈)aryl; or (C₆₋₁₈)aryl having at least one substituent selected from (C₁₋₁₀)alkyl, (C₄₋₁₀)branched alkyl, —O—(C₁-C₁₀)alkyl, —S—(C₁-C₁₀)alkyl, —NRR, —NO₂, —CN, —C(O)R, —C(S)R, —C(O)OR, —C(S)OR, —SC(S)R, —OC(S)R, —C(O)NRR, —C(S)NRR, —C(O)NR(OR), —C(S)NR(OR), —C(O)NR(SR), —C(S)NR(SR), —CH(CN)₂, —CH[C(O)R]₂, —CH[C(S)R]₂, —CH[C(O)OR]₂, —CH[C(S)OR]₂, —CH[C(O)SR]₂, —CH[C(S)SR]₂, —NRC(O)R, —NRC(O)OR, —S(O)—R, —S(O)OR, and —S(O)NRR; or
R₂ and R₃ are linked together to form a nitrogen containing ring system, wherein the nitrogen containing ring system is optionally substituted by at least one substituent selected from (C₁₋₁₀)alkyl, (C₄₋₁₀)branched alkyl, —O—(C₁-C₁₀)alkyl, —S—(C₁-C₁₀)alkyl, —NRR, —NO₂, —CN, —C(O)R, —C(S)R, —C(O)OR, —C(S)OR, —SC(S)R, —OC(S)R, —C(O)NRR, —C(S)NRR, —C(O)NR(OR), —C(S)NR(OR), —C(O)NR(SR), —C(S)NR(SR), —CH(CN)₂, —CH[C(O)R]₂, —CH[C(S)R]₂, —CH[C(O)OR]₂, —CH[C(S)OR]₂, —CH[C(O)SR]₂, —CH[C(S)SR]₂, —NRC(O)R, —NRC(O)OR, —S(O)—R, —S(O)OR, and —S(O)NRR;
wherein:
A is optionally substituted with one or more substituents selected from the group of (C₁-C₁₅)alkyl, (C₂-C₁₅)alkenyl, (C₂-C₁₅)alkynyl, —O—(C₁-C₁₅)alkyl, —O—(C₂-C₁₅) alkenyl, —O—(C₂-C₁₅) alkynyl, —S—(C₁-C₁₅)alkyl, —S—(C₂-C₁₅)alkenyl, —S—(C₂-C₁₅)alkynyl, (C₃-C₈)cycloalkyl, —O—(C₃-C₈)cycloalkyl, —S—(C₃-C₈)cycloalkyl, -halo, —NRR, —NO₂, —CN, —C(O)R, —C(S)R, —C(O)OR, —C(S)OR, —SC(S)R, —OC(S)R, —C(O)NRR, —C(S)NRR, —C(O)NR(OR), —C(S)NR(OR), —C(O)NR(SR), —C(S)NR(SR), —CH(CN)₂, —CH[C(O)R]₂, —CH[C(S)R]₂, —CH[C(O)OR]₂, —CH[C(S)OR]₂, —CH[C(O)SR]₂, —CH[C(S)SR]₂, —NRC(O)R, —NRC(O)OR, —S(O)—R, —S(O)OR, and —S(O)NRR;
wherein
each R is independently selected from —H, (C₁-C₁₀)alkyl, (C₂-C₁₀)alkenyl, (C₂-C₁₀)alkynyl, (C₃-C₈)cycloalkyl, aryl or substituted aryl;
wherein
when Y and/or Z are O or S, R₂ and/or R₃ are absent;
or a pharmaceutically acceptable salt, a prodrug, a tautomer or a solvate thereof.

9. A compound of Formula II: 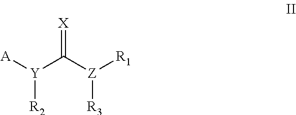
wherein:
A is an arene or a heteroarene;
Y is N, O or S;
Z is N, O or S;
X is O, S or N═CN;
R₁ is a (C₄₋₁₅)-branched alkyl; (C₃₋₈)cycloalkyl; (C₃₋₈)cycloalkyl having at least one substituent selected from (C₁₋₁₀)alkyl, (C₄₋₁₀)branched alkyl, —O—(C₁-C₁₀)alkyl, —S—(C₁-C₁₀)alkyl, —NRR, —NO₂, —CN, —C(O)R, —C(S)R, —C(O)OR, —C(S)OR, —SC(S)R, —OC(S)R, —C(O)NRR, —C(S)NRR, —C(O)NR(OR), —C(S)NR(OR), —C(O)NR(SR), —C(S)NR(SR), —CH(CN)₂, —CH[C(O)R]₂, —CH[C(S)R]₂, —CH[C(O)OR]₂, —CH[C(S)OR]₂, —CH[C(O)SR]₂, —CH[C(S)SR]₂, —NRC(O)R, —NRC(O)OR, —S(O)—R, —S(O)OR, and —S(O)NRR; (C₆₋₁₈)aryl; or (C₆₋₁₈)aryl having at least one substituent selected from (C₁₋₁₀)alkyl, (C₄₋₁₀)branched alkyl, —O—(C₁-C₁₀)alkyl, —S—(C₁-C₁₀)alkyl, —NRR, —NO₂, —CN, —C(O)R, —C(S)R, —C(O)OR, —C(S)OR, —SC(S)R, —OC(S)R, —C(O)NRR, —C(S)NRR, —C(O)NR(OR), —C(S)NR(OR), —C(O)NR(SR), —C(S)NR(SR), —CH(CN)₂, —CH[C(O)R]₂, —CH[C(S)R]₂, —CH[C(O)OR]₂, —CH[C(S)OR]₂, —CH[C(O)SR]₂, —CH[C(S)SR]₂, —NRC(O)R, —NRC(O)OR, —S(O)—R, —S(O)OR, and —S(O)NRR;
R₂ and R₃ are independently hydrogen, (C₁₋₁₀)alkyl, (C₄₋₁₀)branched alkyl, (C₃₋₈)cycloalkyl; (C₃₋₈)cycloalkyl having at least one substituent selected from (C₁₋₁₀)alkyl, (C₄₋₁₀)branched alkyl, —O—(C₁-C₁₀)alkyl, —S—(C₁-C₁₀)alkyl, —NRR, —NO₂, —CN, —C(O)R, —C(S)R, —C(O)OR, —C(S)OR, —SC(S)R, —OC(S)R, —C(O)NRR, —C(S)NRR, —C(O)NR(OR), —C(S)NR(OR), —C(O)NR(SR), —C(S)NR(SR), —CH(CN)₂, —CH[C(O)R]₂, —CH[C(S)R]₂, —CH[C(O)OR]₂, —CH[C(S)OR]₂, —CH[C(O)SR]₂, —CH[C(S)SR]₂, —NRC(O)R, —NRC(O)OR, —S(O)—R, —S(O)OR, and —S(O)NRR; (C₆₋₁₈)aryl; or (C₆₋₁₈)aryl having at least one substituent selected from (C₁₋₁₀)alkyl, (C₄₋₁₀)branched alkyl, —O—(C₁-C₁₀)alkyl, —S—(C₁-C₁₀)alkyl, —NRR, —NO₂, —CN, —C(O)R, —C(S)R, —C(O)OR, —C(S)OR, —SC(S)R, —OC(S)R, —C(O)NRR, —C(S)NRR, —C(O)NR(OR), —C(S)NR(OR), —C(O)NR(SR), —C(S)NR(SR), —CH(CN)₂, —CH[C(O)R]₂, —CH[C(S)R]₂, —CH[C(O)OR]₂, —CH[C(S)OR]₂, —CH[C(O)SR]₂, —CH[C(S)SR]₂, —NRC(O)R, —NRC(O)OR, —S(O)—R, —S(O)OR, and —S(O)NRR; or
R₂ and R₃ are linked together to form a nitrogen containing ring system, wherein the nitrogen containing ring system is optionally substituted by at least one substituent selected from (C₁₋₁₀)alkyl, (C₄₋₁₀)branched alkyl, —O—(C₁-C₁₀)alkyl, —S—(C₁-C₁₀)alkyl, —NRR, —NO₂, —CN, —C(O)R, —C(S)R, —C(O)OR, —C(S)OR, —SC(S)R, —OC(S)R, —C(O)NRR, —C(S)NRR, —C(O)NR(OR), —C(S)NR(OR), —C(O)NR(SR), —C(S)NR(SR), —CH(CN)₂, —CH[C(O)R]₂, —CH[C(S)R]₂, —CH[C(O)OR]₂, —CH[C(S)OR]₂, —CH[C(O)SR]₂, —CH[C(S)SR]₂, —NRC(O)R, —NRC(O)OR, —S(O)—R, —S(O)OR, and —S(O)NRR;
wherein:
A is optionally substituted with one or more substituents selected from the group of (C₁-C₁₅)alkyl, (C₂-C₁₅)alkenyl, (C₂-C₁₅)alkynyl, —O—(C₁-C₁₅)alkyl, —O—(C₂-C₁₅) alkenyl, —O—(C₂-C₁₅) alkynyl, —S—(C₁-C₁₅)alkyl, —S—(C₂-C₁₅)alkenyl, —S—(C₂-C₁₅)alkynyl, (C₃-C₈)cycloalkyl, —O—(C₃-C₈)cycloalkyl, —S—(C₃-C₈)cycloalkyl, -halo, —NRR, —NO₂, —CN, —C(O)R, —C(S)R, —C(O)OR, —C(S)OR, —SC(S)R, —OC(S)R, —C(O)NRR, —C(S)NRR, —C(O)NR(OR), —C(S)NR(OR), —C(O)NR(SR), —C(S)NR(SR), —CH(CN)₂, —CH[C(O)R]₂, —CH[C(S)R]₂, —CH[C(O)OR]₂, —CH[C(S)OR]₂, —CH[C(O)SR]₂, —CH[C(S)SR]₂, —NRC(O)R, —NRC(O)OR, —S(O)—R, —S(O)OR, and —S(O)NRR;
wherein
each R is independently selected from —H, (C₁-C₁₀)alkyl, (C₂-C₁₀)alkenyl, (C₂-C₁₀)alkynyl, (C₃-C₈)cycloalkyl, aryl or substituted aryl;
wherein
when Y and/or Z are O or S, R₂ and/or R₃ are absent;
or a pharmaceutically acceptable salt, a prodrug, a tautomer or a solvate thereof;
for use as an anti-proliferative agent in the attenuation, inhibition, or prevention of conditions associated with IL-6 expression.

10. A compound of Formula II: 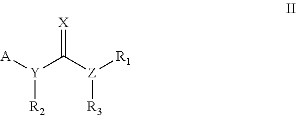
wherein:
A is an arene or a heteroarene;
Y is N, O or S;
Z is N, O or S;
X is O, S or N═CN;
R₁ is a (C₄₋₁₅)-branched alkyl; (C₃₋₈)cycloalkyl; (C₃₋₈)cycloalkyl having at least one substituent selected from (C₁₋₁₀)alkyl, (C₄₋₁₀)branched alkyl, —O—(C₁-C₁₀)alkyl, —S—(C₁-C₁₀)alkyl, —NRR, —NO₂, —CN, —C(O)R, —C(S)R, —C(O)OR, —C(S)OR, —SC(S)R, —OC(S)R, —C(O)NRR, —C(S)NRR, —C(O)NR(OR), —C(S)NR(OR), —C(O)NR(SR), —C(S)NR(SR), —CH(CN)₂, —CH[C(O)R]₂, —CH[C(S)R]₂, —CH[C(O)OR]₂, —CH[C(S)OR]₂, —CH[C(O)SR]₂, —CH[C(S)SR]₂, —NRC(O)R, —NRC(O)OR, —S(O)—R, —S(O)OR, and —S(O)NRR; (C₆₋₁₈)aryl; or (C₆₋₁₈)aryl having at least one substituent selected from (C₁₋₁₀)alkyl, (C₄₋₁₀)branched alkyl, —O—(C₁-C₁₀)alkyl, —S—(C₁-C₁₀)alkyl, —NRR, —NO₂, —CN, —C(O)R, —C(S)R, —C(O)OR, —C(S)OR, —SC(S)R, —OC(S)R, —C(O)NRR, —C(S)NRR, —C(O)NR(OR), —C(S)NR(OR), —C(O)NR(SR), —C(S)NR(SR), —CH(CN)₂, —CH[C(O)R]₂, —CH[C(S)R]₂, —CH[C(O)OR]₂, —CH[C(S)OR]₂, —CH[C(O)SR]₂, —CH[C(S)SR]₂, —NRC(O)R, —NRC(O)OR, —S(O)—R, —S(O)OR, and —S(O)NRR;
R₂ and R₃ are independently hydrogen, (C₁₋₁₀)alkyl, (C₄₋₁₀)branched alkyl, (C₃₋₈)cycloalkyl; (C₃₋₈)cycloalkyl having at least one substituent selected from (C₁₋₁₀)alkyl, (C₄₋₁₀)branched alkyl, —O—(C₁-C₁₀)alkyl, —S—(C₁-C₁₀)alkyl, —NRR, —NO₂, —CN, —C(O)R, —C(S)R, —C(O)OR, —C(S)OR, —SC(S)R, —OC(S)R, —C(O)NRR, —C(S)NRR, —C(O)NR(OR), —C(S)NR(OR), —C(O)NR(SR), —C(S)NR(SR), —CH(CN)₂, —CH[C(O)R]₂, —CH[C(S)R]₂, —CH[C(O)OR]₂, —CH[C(S)OR]₂, —CH[C(O)SR]₂, —CH[C(S)SR]₂, —NRC(O)R, —NRC(O)OR, —S(O)—R, —S(O)OR, and —S(O)NRR; (C₆₋₁₈)aryl; or (C₆₋₁₈)aryl having at least one substituent selected from (C₁₋₁₀)alkyl, (C₄₋₁₀)branched alkyl, —O—(C₁-C₁₀)alkyl, —S—(C₁-C₁₀)alkyl, —NRR, —NO₂, —CN, —C(O)R, —C(S)R, —C(O)OR, —C(S)OR, —SC(S)R, —OC(S)R, —C(O)NRR, —C(S)NRR, —C(O)NR(OR), —C(S)NR(OR), —C(O)NR(SR), —C(S)NR(SR), —CH(CN)₂, —CH[C(O)R]₂, —CH[C(S)R]₂, —CH[C(O)OR]₂, —CH[C(S)OR]₂, —CH[C(O)SR]₂, —CH[C(S)SR]₂, —NRC(O)R, —NRC(O)OR, —S(O)—R, —S(O)OR, and —S(O)NRR; or
R₂ and R₃ are linked together to form a nitrogen containing ring system, wherein the nitrogen containing ring system is optionally substituted by at least one substituent selected from (C₁₋₁₀)alkyl, (C₄₋₁₀)branched alkyl, —O—(C₁-C₁₀)alkyl, —S—(C₁-C₁₀)alkyl, —NRR, —NO₂, —CN, —C(O)R, —C(S)R, —C(O)OR, —C(S)OR, —SC(S)R, —OC(S)R, —C(O)NRR, —C(S)NRR, —C(O)NR(OR), —C(S)NR(OR), —C(O)NR(SR), —C(S)NR(SR), —CH(CN)₂, —CH[C(O)R]₂, —CH[C(S)R]₂, —CH[C(O)OR]₂, —CH[C(S)OR]₂, —CH[C(O)SR]₂, —CH[C(S)SR]₂, —NRC(O)R, —NRC(O)OR, —S(O)—R, —S(O)OR, and —S(O)NRR;
wherein:
A is optionally substituted with one or more substituents selected from the group of (C₁-C₁₅)alkyl, (C₂-C₁₅)alkenyl, (C₂-C₁₅)alkynyl, —O—(C₁-C₁₅)alkyl, —O—(C₂-C₁₅) alkenyl, —O—(C₂-C₁₅) alkynyl, —S—(C₁-C₁₅)alkyl, —S—(C₂-C₁₅)alkenyl, —S—(C₂-C₁₅)alkynyl, (C₃-C₈)cycloalkyl, —O—(C₃-C₈)cycloalkyl, —S—(C₃-C₈)cycloalkyl, -halo, —NRR, —NO₂, —CN, —C(O)R, —C(S)R, —C(O)OR, —C(S)OR, —SC(S)R, —OC(S)R, —C(O)NRR, —C(S)NRR, —C(O)NR(OR), —C(S)NR(OR), —C(O)NR(SR), —C(S)NR(SR), —CH(CN)₂, —CH[C(O)R]₂, —CH[C(S)R]₂, —CH[C(O)OR]₂, —CH[C(S)OR]₂, —CH[C(O)SR]₂, —CH[C(S)SR]₂, —NRC(O)R, —NRC(O)OR, —S(O)—R, —S(O)OR, and —S(O)NRR;
wherein
each R is independently selected from —H, (C₁-C₁₀)alkyl, (C₂-C₁₀)alkenyl, (C₂-C₁₀)alkynyl, (C₃-C₈)cycloalkyl, aryl or substituted aryl;
wherein
when Y and/or Z are O or S, R₂ and/or R₃ are absent;
or a pharmaceutically acceptable salt, a prodrug, a tautomer or a solvate thereof;
for use as a therapeutic agent in the attenuation, inhibition, or prevention of conditions associated with IL-6 expression.

11. A method for treating, attenuating, inhibiting, or preventing a condition associated with IL-6 expression in a subject in need thereof, the method comprising administering a therapeutically effective amount of a compound of Formula II: 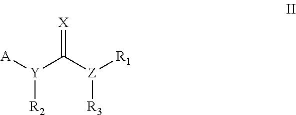
wherein:
A is an arene or a heteroarene;
Y is N, O or S;
Z is N, O or S;
X is O, S or N═CN;
R₁ is a (C₄₋₁₅)-branched alkyl; (C₃₋₈)cycloalkyl; (C₃₋₈)cycloalkyl having at least one substituent selected from (C₁₋₁₀)alkyl, (C₄₋₁₀)branched alkyl, —O—(C₁-C₁₀)alkyl, —S—(C₁-C₁₀)alkyl, —NRR, —NO₂, —CN, —C(O)R, —C(S)R, —C(O)OR, —C(S)OR, —SC(S)R, —OC(S)R, —C(O)NRR, —C(S)NRR, —C(O)NR(OR), —C(S)NR(OR), —C(O)NR(SR), —C(S)NR(SR), —CH(CN)₂, —CH[C(O)R]₂, —CH[C(S)R]₂, —CH[C(O)OR]₂, —CH[C(S)OR]₂, —CH[C(O)SR]₂, —CH[C(S)SR]₂, —NRC(O)R, —NRC(O)OR, —S(O)—R, —S(O)OR, and —S(O)NRR; (C₆₋₁₈)aryl; or (C₆₋₁₈)aryl having at least one substituent selected from (C₁₋₁₀)alkyl, (C₄₋₁₀)branched alkyl, —O—(C₁-C₁₀)alkyl, —S—(C₁-C₁₀)alkyl, —NRR, —NO₂, —CN, —C(O)R, —C(S)R, —C(O)OR, —C(S)OR, —SC(S)R, —OC(S)R, —C(O)NRR, —C(S)NRR, —C(O)NR(OR), —C(S)NR(OR), —C(O)NR(SR), —C(S)NR(SR), —CH(CN)₂, —CH[C(O)R]₂, —CH[C(S)R]₂, —CH[C(O)OR]₂, —CH[C(S)OR]₂, —CH[C(O)SR]₂, —CH[C(S)SR]₂, —NRC(O)R, —NRC(O)OR, —S(O)—R, —S(O)OR, and —S(O)NRR;
R₂ and R₃ are independently hydrogen, (C₁₋₁₀)alkyl, (C₄₋₁₀)branched alkyl, (C₃₋₈)cycloalkyl; (C₃₋₈)cycloalkyl having at least one substituent selected from (C₁₋₁₀)alkyl, (C₄₋₁₀)branched alkyl, —O—(C₁-C₁₀)alkyl, —S—(C₁-C₁₀)alkyl, —NRR, —NO₂, —CN, —C(O)R, —C(S)R, —C(O)OR, —C(S)OR, —SC(S)R, —OC(S)R, —C(O)NRR, —C(S)NRR, —C(O)NR(OR), —C(S)NR(OR), —C(O)NR(SR), —C(S)NR(SR), —CH(CN)₂, —CH[C(O)R]₂, —CH[C(S)R]₂, —CH[C(O)OR]₂, —CH[C(S)OR]₂, —CH[C(O)SR]₂, —CH[C(S)SR]₂, —NRC(O)R, —NRC(O)OR, —S(O)—R, —S(O)OR, and —S(O)NRR; (C₆₋₁₈)aryl; or (C₆₋₁₈)aryl having at least one substituent selected from (C₁₋₁₀)alkyl, (C₄₋₁₀)branched alkyl, —O—(C₁-C₁₀)alkyl, —S—(C₁-C₁₀)alkyl, —NRR, —NO₂, —CN, —C(O)R, —C(S)R, —C(O)OR, —C(S)OR, —SC(S)R, —OC(S)R, —C(O)NRR, —C(S)NRR, —C(O)NR(OR), —C(S)NR(OR), —C(O)NR(SR), —C(S)NR(SR), —CH(CN)₂, —CH[C(O)R]₂, —CH[C(S)R]₂, —CH[C(O)OR]₂, —CH[C(S)OR]₂, —CH[C(O)SR]₂, —CH[C(S)SR]₂, —NRC(O)R, —NRC(O)OR, —S(O)—R, —S(O)OR, and —S(O)NRR; or
R₂ and R₃ are linked together to form a nitrogen containing ring system, wherein the nitrogen containing ring system is optionally substituted by at least one substituent selected from (C₁₋₁₀)alkyl, (C₄₋₁₀)branched alkyl, —O—(C₁-C₁₀)alkyl, —S—(C₁-C₁₀)alkyl, —NRR, —NO₂, —CN, —C(O)R, —C(S)R, —C(O)OR, —C(S)OR, —SC(S)R, —OC(S)R, —C(O)NRR, —C(S)NRR, —C(O)NR(OR), —C(S)NR(OR), —C(O)NR(SR), —C(S)NR(SR), —CH(CN)₂, —CH[C(O)R]₂, —CH[C(S)R]₂, —CH[C(O)OR]₂, —CH[C(S)OR]₂, —CH[C(O)SR]₂, —CH[C(S)SR]₂, —NRC(O)R, —NRC(O)OR, —S(O)—R, —S(O)OR, and —S(O)NRR;
wherein:
A is optionally substituted with one or more substituents selected from the group of (C₁-C₁₅)alkyl, (C₂-C₁₅)alkenyl, (C₂-C₁₅)alkynyl, —O—(C₁-C₁₅)alkyl, —O—(C₂-C₁₅) alkenyl, —O—(C₂-C₁₅) alkynyl, —S—(C₁-C₁₅)alkyl, —S—(C₂-C₁₅)alkenyl, —S—(C₂-C₁₅)alkynyl, (C₃-C₈)cycloalkyl, —O—(C₃-C₈)cycloalkyl, —S—(C₃-C₈)cycloalkyl, -halo, —NRR, —NO₂, —CN, —C(O)R, —C(S)R, —C(O)OR, —C(S)OR, —SC(S)R, —OC(S)R, —C(O)NRR, —C(S)NRR, —C(O)NR(OR), —C(S)NR(OR), —C(O)NR(SR), —C(S)NR(SR), —CH(CN)₂, —CH[C(O)R]₂, —CH[C(S)R]₂, —CH[C(O)OR]₂, —CH[C(S)OR]₂, —CH[C(O)SR]₂, —CH[C(S)SR]₂, —NRC(O)R, —NRC(O)OR, —S(O)—R, —S(O)OR, and —S(O)NRR;
wherein
each R is independently selected from —H, (C₁-C₁₀)alkyl, (C₂-C₁₀)alkenyl, (C₂-C₁₀)alkynyl, (C₃-C₈)cycloalkyl, aryl or substituted aryl;
wherein
when Y and/or Z are O or S, R₂ and/or R₃ are absent;
or a pharmaceutically acceptable salt, a prodrug, a tautomer or a solvate thereof.

12. A pharmaceutical composition comprising a compound as defined in any one of claims 8 to 10, and a pharmaceutically acceptable carrier.

13. A medicament for use in treating, attenuating, inhibiting and/or preventing inflammation and inflammation-related pathologies, the medicament comprising a compound as defined in any one of claims 8 to 10, and a pharmaceutically acceptable carrier.

14. A medicament for use in treating a condition associated with IL-6 expression, the medicament comprising a compound as defined in any one of claims 8 to 10, and a pharmaceutically acceptable carrier.

15. A compound of Formula III: 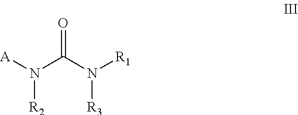
wherein:
A is an arene or a heteroarene;
R₁ is a (C₄₋₁₅)-branched alkyl; (C₃₋₈)cycloalkyl; (C₃₋₈)cycloalkyl having at least one substituent selected from (C₁₋₁₀)alkyl, (C₄₋₁₀)branched alkyl, —O—(C₁-C₁₀)alkyl, —S—(C₁-C₁₀)alkyl, —NRR, —NO₂, —CN, —C(O)R, —C(S)R, —C(O)OR, —C(S)OR, —SC(S)R, —OC(S)R, —C(O)NRR, —C(S)NRR, —C(O)NR(OR), —C(S)NR(OR), —C(O)NR(SR), —C(S)NR(SR), —CH(CN)₂, —CH[C(O)R]₂, —CH[C(S)R]₂, —CH[C(O)OR]₂, —CH[C(S)OR]₂, —CH[C(O)SR]₂, —CH[C(S)SR]₂, —NRC(O)R, —NRC(O)OR, —S(O)—R, —S(O)OR, and —S(O)NRR; (C₆₋₁₈)aryl; or (C₆₋₁₈)aryl having at least one substituent selected from (C₁₋₁₀)alkyl, (C₄₋₁₀)branched alkyl, —O—(C₁-C₁₀)alkyl, —S—(C₁-C₁₀)alkyl, —NRR, —NO₂, —CN, —C(O)R, —C(S)R, —C(O)OR, —C(S)OR, —SC(S)R, —OC(S)R, —C(O)NRR, —C(S)NRR, —C(O)NR(OR), —C(S)NR(OR), —C(O)NR(SR), —C(S)NR(SR), —CH(CN)₂, —CH[C(O)R]₂, —CH[C(S)R]₂, —CH[C(O)OR]₂, —CH[C(S)OR]₂, —CH[C(O)SR]₂, —CH[C(S)SR]₂, —NRC(O)R, —NRC(O)OR, —S(O)—R, —S(O)OR, and —S(O)NRR;
R₂ and R₃ are independently hydrogen, (C₁₋₁₀)alkyl, (C₄₋₁₀)branched alkyl, (C₃₋₈)cycloalkyl; (C₃₋₈)cycloalkyl having at least one substituent selected from (C₁₋₁₀)alkyl, (C₄₋₁₀)branched alkyl, —O—(C₁-C₁₀)alkyl, —S—(C₁-C₁₀)alkyl, —NRR, —NO₂, —CN, —C(O)R, —C(S)R, —C(O)OR, —C(S)OR, —SC(S)R, —OC(S)R, —C(O)NRR, —C(S)NRR, —C(O)NR(OR), —C(S)NR(OR), —C(O)NR(SR), —C(S)NR(SR), —CH(CN)₂, —CH[C(O)R]₂, —CH[C(S)R]₂, —CH[C(O)OR]₂, —CH[C(S)OR]₂, —CH[C(O)SR]₂, —CH[C(S)SR]₂, —NRC(O)R, —NRC(O)OR, —S(O)—R, —S(O)OR, and —S(O)NRR; (C₆₋₁₈)aryl; or (C₆₋₁₈)aryl having at least one substituent selected from (C₁₋₁₀)alkyl, (C₄₋₁₀)branched alkyl, —O—(C₁-C₁₀)alkyl, —S—(C₁-C₁₀)alkyl, —NRR, —NO₂, —CN, —C(O)R, —C(S)R, —C(O)OR, —C(S)OR, —SC(S)R, —OC(S)R, —C(O)NRR, —C(S)NRR, —C(O)NR(OR), —C(S)NR(OR), —C(O)NR(SR), —C(S)NR(SR), —CH(CN)₂, —CH[C(O)R]₂, —CH[C(S)R]₂, —CH[C(O)OR]₂, —CH[C(S)OR]₂, —CH[C(O)SR]₂, —CH[C(S)SR]₂, —NRC(O)R, —NRC(O)OR, —S(O)—R, —S(O)OR, and —S(O)NRR; or
R₂ and R₃ are linked together to form a nitrogen containing ring system, wherein the nitrogen containing ring system is optionally substituted by at least one substituent selected from (C₁₋₁₀)alkyl, (C₄₋₁₀)branched alkyl, —O—(C₁-C₁₀)alkyl, —S—(C₁-C₁₀)alkyl, —NRR, —NO₂, —CN, —C(O)R, —C(S)R, —C(O)OR, —C(S)OR, —SC(S)R, —OC(S)R, —C(O)NRR, —C(S)NRR, —C(O)NR(OR), —C(S)NR(OR), —C(O)NR(SR), —C(S)NR(SR), —CH(CN)₂, —CH[C(O)R]₂, —CH[C(S)R]₂, —CH[C(O)OR]₂, —CH[C(S)OR]₂, —CH[C(O)SR]₂, —CH[C(S)SR]₂, —NRC(O)R, —NRC(O)OR, —S(O)—R, —S(O)OR, and —S(O)NRR;
wherein:
A is optionally substituted with one or more substituents selected from the group of (C₁-C₁₅)alkyl, (C₂-C₁)alkenyl, (C₂-C₁₅)alkynyl, —O—(C₁-C₁₅)alkyl, —O—(C₂-C₁₅) alkenyl, —O—(C₂-C₁₅) alkynyl, —S—(C₁-C₁₅)alkyl, —S—(C₂-C₁₅)alkenyl, —S—(C₂-C₅)alkynyl, (C₃-C₈)cycloalkyl, —O—(C₃-C₈)cycloalkyl, —S—(C₃-C₈)cycloalkyl, -halo, —NRR, —NO₂, —CN, —C(O)R, —C(S)R, —C(O)OR, —C(S)OR, —SC(S)R, —OC(S)R, —C(O)NRR, —C(S)NRR, —C(O)NR(OR), —C(S)NR(OR), —C(O)NR(SR), —C(S)NR(SR), —CH(CN)₂, —CH[C(O)R]₂, —CH[C(S)R]₂, —CH[C(O)OR]₂, —CH[C(S)OR]₂, —CH[C(O)SR]₂, —CH[C(S)SR]₂, —NRC(O)R, —NRC(O)OR, —S(O)—R, —S(O)OR, and —S(O)NRR;
wherein:
each R is independently selected from —H, (C₁-C₁₀)alkyl, (C₂-C₁₀)alkenyl, (C₂-C₁₀)alkynyl, (C₃-C₈)cycloalkyl, aryl or substituted aryl;
or a pharmaceutically acceptable salt, a prodrug, a tautomer or a solvate thereof.

16. A compound of Formula III: 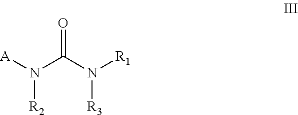
wherein:
A is an arene or a heteroarene;
R₁ is a (C₄₋₁₅)-branched alkyl; (C₃₋₈)cycloalkyl; (C₃₋₈)cycloalkyl having at least one substituent selected from (C₁₋₁₀)alkyl, (C₄₋₁₀)branched alkyl, —O—(C₁-C₀)alkyl, —S—(C₁-C₁₀)alkyl, —NRR, —NO₂, —CN, —C(O)R, —C(S)R, —C(O)OR, —C(S)OR, —SC(S)R, —OC(S)R, —C(O)NRR, —C(S)NRR, —C(O)NR(OR), —C(S)NR(OR), —C(O)NR(SR), —C(S)NR(SR), —CH(CN)₂, —CH[C(O)R]₂, —CH[C(S)R]₂, —CH[C(O)OR]₂, —CH[C(S)OR]₂, —CH[C(O)SR]₂, —CH[C(S)SR]₂, —NRC(O)R, —NRC(O)OR, —S(O)—R, —S(O)OR, and —S(O)NRR; (C₆₋₁₈)aryl; or (C₆₋₁₈)aryl having at least one substituent selected from (C₁₋₁₀)alkyl, (C₄₋₁₀)branched alkyl, —O—(C₁-C₁₀)alkyl, —S—(C₁-C₁₀)alkyl, —NRR, —NO₂, —CN, —C(O)R, —C(S)R, —C(O)OR, —C(S)OR, —SC(S)R, —OC(S)R, —C(O)NRR, —C(S)NRR, —C(O)NR(OR), —C(S)NR(OR), —C(O)NR(SR), —C(S)NR(SR), —CH(CN)₂, —CH[C(O)R]₂, —CH[C(S)R]₂, —CH[C(O)OR]₂, —CH[C(S)OR]₂, —CH[C(O)SR]₂, —CH[C(S)SR]₂, —NRC(O)R, —NRC(O)OR, —S(O)—R, —S(O)OR, and —S(O)NRR;
R₂ and R₃ are independently hydrogen, (C₁₋₁₀)alkyl, (C₄₋₁₀)branched alkyl, (C₃₋₈)cycloalkyl; (C₃₋₈)cycloalkyl having at least one substituent selected from (C₁₋₁₀)alkyl, (C₄₋₁₀)branched alkyl, —O—(C₁-C₁₀)alkyl, —S—(C₁-C₁₀)alkyl, —NRR, —NO₂, —CN, —C(O)R, —C(S)R, —C(O)OR, —C(S)OR, —SC(S)R, —OC(S)R, —C(O)NRR, —C(S)NRR, —C(O)NR(OR), —C(S)NR(OR), —C(O)NR(SR), —C(S)NR(SR), —CH(CN)₂, —CH[C(O)R]₂, —CH[C(S)R]₂, —CH[C(O)OR]₂, —CH[C(S)OR]₂, —CH[C(O)SR]₂, —CH[C(S)SR]₂, —NRC(O)R, —NRC(O)OR, —S(O)—R, —S(O)OR, and —S(O)NRR; (C₆₋₁₈)aryl; or (C₆₋₁₈)aryl having at least one substituent selected from (C₁₋₁₀)alkyl, (C₄₋₁₀)branched alkyl, —O—(C₁-C₁₀)alkyl, —S—(C₁-C₁₀)alkyl, —NRR, —NO₂, —CN, —C(O)R, —C(S)R, —C(O)OR, —C(S)OR, —SC(S)R, —OC(S)R, —C(O)NRR, —C(S)NRR, —C(O)NR(OR), —C(S)NR(OR), —C(O)NR(SR), —C(S)NR(SR), —CH(CN)₂, —CH[C(O)R]₂, —CH[C(S)R]₂, —CH[C(O)OR]₂, —CH[C(S)OR]₂, —CH[C(O)SR]₂, —CH[C(S)SR]₂, —NRC(O)R, —NRC(O)OR, —S(O)—R, —S(O)OR, and —S(O)NRR; or
R₂ and R₃ are linked together to form a nitrogen containing ring system, wherein the nitrogen containing ring system is optionally substituted by at least one substituent selected from (C₁₋₁₀)alkyl, (C₄₋₁₀)branched alkyl, —O—(C₁-C₁₀)alkyl, —S—(C₁-C₁₀)alkyl, —NRR, —NO₂, —CN, —C(O)R, —C(S)R, —C(O)OR, —C(S)OR, —SC(S)R, —OC(S)R, —C(O)NRR, —C(S)NRR, —C(O)NR(OR), —C(S)NR(OR), —C(O)NR(SR), —C(S)NR(SR), —CH(CN)₂, —CH[C(O)R]₂, —CH[C(S)R]₂, —CH[C(O)OR]₂, —CH[C(S)OR]₂, —CH[C(O)SR]₂, —CH[C(S)SR]₂, —NRC(O)R, —NRC(O)OR, —S(O)—R, —S(O)OR, and —S(O)NRR;
wherein:
A is optionally substituted with one or more substituents selected from the group of (C₁-C₁₅)alkyl, (C₂-C₁₅)alkenyl, (C₂-C₁₅)alkynyl, —O—(C₁-C₁₅)alkyl, —O—(C₂-C₁₅) alkenyl, —O—(C₂-C₁₅) alkynyl, —S—(C₁-C₁₅)alkyl, —S—(C₂-C₁₅)alkenyl, —S—(C₂-C₁₅)alkynyl, (C₃-C₈)cycloalkyl, —O—(C₃-C₈)cycloalkyl, —S—(C₃-C₈)cycloalkyl, -halo, —NRR, —NO₂, —CN, —C(O)R, —C(S)R, —C(O)OR, —C(S)OR, —SC(S)R, —OC(S)R, —C(O)NRR, —C(S)NRR, —C(O)NR(OR), —C(S)NR(OR), —C(O)NR(SR), —C(S)NR(SR), —CH(CN)₂, —CH[C(O)R]₂, —CH[C(S)R]₂, —CH[C(O)OR]₂, —CH[C(S)OR]₂, —CH[C(O)SR]₂, —CH[C(S)SR]₂, —NRC(O)R, —NRC(O)OR, —S(O)—R, —S(O)OR, and —S(O)NRR;
wherein:
each R is independently selected from —H, (C₁-C₁₀)alkyl, (C₂-C₁₀)alkenyl, (C₂-C₁₀)alkynyl, (C₃-C₈)cycloalkyl, aryl or substituted aryl;
or a pharmaceutically acceptable salt, a prodrug, a tautomer or a solvate thereof;
for use as an anti-proliferative agent in the attenuation, inhibition, or prevention of conditions associated with IL-6 expression.

17. A compound of Formula III: 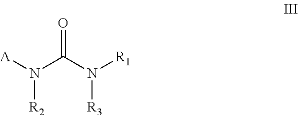
wherein:
A is an arene or a heteroarene;
R₁ is a (C₄₋₁₅)-branched alkyl; (C₃₋₈)cycloalkyl; (C₃₋₈)cycloalkyl having at least one substituent selected from (C₁₋₁₀)alkyl, (C₄₋₁₀)branched alkyl, —O—(C₁-C₁₀)alkyl, —S—(C₁-C₁₀)alkyl, —NRR, —NO₂, —CN, —C(O)R, —C(S)R, —C(O)OR, —C(S)OR, —SC(S)R, —OC(S)R, —C(O)NRR, —C(S)NRR, —C(O)NR(OR), —C(S)NR(OR), —C(O)NR(SR), —C(S)NR(SR), —CH(CN)₂, —CH[C(O)R]₂, —CH[C(S)R]₂, —CH[C(O)OR]₂, —CH[C(S)OR]₂, —CH[C(O)SR]₂, —CH[C(S)SR]₂, —NRC(O)R, —NRC(O)OR, —S(O)—R, —S(O)OR, and —S(O)NRR; (C₆₋₁₈)aryl; or (C₆₋₁₈)aryl having at least one substituent selected from (C₁₋₁₀)alkyl, (C₄₋₁₀)branched alkyl, —O—(C₁-C₁₀)alkyl, —S—(C₁-C₁₀)alkyl, —NRR, —NO₂, —CN, —C(O)R, —C(S)R, —C(O)OR, —C(S)OR, —SC(S)R, —OC(S)R, —C(O)NRR, —C(S)NRR, —C(O)NR(OR), —C(S)NR(OR), —C(O)NR(SR), —C(S)NR(SR), —CH(CN)₂, —CH[C(O)R]₂, —CH[C(S)R]₂, —CH[C(O)OR]₂, —CH[C(S)OR]₂, —CH[C(O)SR]₂, —CH[C(S)SR]₂, —NRC(O)R, —NRC(O)OR, —S(O)—R, —S(O)OR, and —S(O)NRR;
R₂ and R₃ are independently hydrogen, (C₁₋₁₀)alkyl, (C₄₋₁₀)branched alkyl, (C₃₋₈)cycloalkyl; (C₃₋₈)cycloalkyl having at least one substituent selected from (C₁₋₁₀)alkyl, (C₄₋₁₀)branched alkyl, —O—(C₁-C₁₀)alkyl, —S—(C₁-C₁₀)alkyl, —NRR, —NO₂, —CN, —C(O)R, —C(S)R, —C(O)OR, —C(S)OR, —SC(S)R, —OC(S)R, —C(O)NRR, —C(S)NRR, —C(O)NR(OR), —C(S)NR(OR), —C(O)NR(SR), —C(S)NR(SR), —CH(CN)₂, —CH[C(O)R]₂, —CH[C(S)R]₂, —CH[C(O)OR]₂, —CH[C(S)OR]₂, —CH[C(O)SR]₂, —CH[C(S)SR]₂, —NRC(O)R, —NRC(O)OR, —S(O)—R, —S(O)OR, and —S(O)NRR; (C₆₋₁₈)aryl; or (C₆₋₁₈)aryl having at least one substituent selected from (C₁₋₁₀)alkyl, (C₄₋₁₀)branched alkyl, —O—(C₁-C₁₀)alkyl, —S—(C₁-C₁₀)alkyl, —NRR, —NO₂, —CN, —C(O)R, —C(S)R, —C(O)OR, —C(S)OR, —SC(S)R, —OC(S)R, —C(O)NRR, —C(S)NRR, —C(O)NR(OR), —C(S)NR(OR), —C(O)NR(SR), —C(S)NR(SR), —CH(CN)₂, —CH[C(O)R]₂, —CH[C(S)R]₂, —CH[C(O)OR]₂, —CH[C(S)OR]₂, —CH[C(O)SR]₂, —CH[C(S)SR]₂, —NRC(O)R, —NRC(O)OR, —S(O)—R, —S(O)OR, and —S(O)NRR; or
R₂ and R₃ are linked together to form a nitrogen containing ring system, wherein the nitrogen containing ring system is optionally substituted by at least one substituent selected from (C₁₋₁₀)alkyl, (C₄₋₁₀)branched alkyl, —O—(C₁-C₁₀)alkyl, —S—(C₁-C₁₀)alkyl, —NRR, —NO₂, —CN, —C(O)R, —C(S)R, —C(O)OR, —C(S)OR, —SC(S)R, —OC(S)R, —C(O)NRR, —C(S)NRR, —C(O)NR(OR), —C(S)NR(OR), —C(O)NR(SR), —C(S)NR(SR), —CH(CN)₂, —CH[C(O)R]₂, —CH[C(S)R]₂, —CH[C(O)OR]₂, —CH[C(S)OR]₂, —CH[C(O)SR]₂, —CH[C(S)SR]₂, —NRC(O)R, —NRC(O)OR, —S(O)—R, —S(O)OR, and —S(O)NRR;
wherein:
A is optionally substituted with one or more substituents selected from the group of (C₁-C₁₅)alkyl, (C₂-C₁₅)alkenyl, (C₂-C₁₅)alkynyl, —O—(C₁-C₁₅)alkyl, —O—(C₂-C₁₅) alkenyl, —O—(C₂-C₁₅) alkynyl, —S—(C₁-C₁₅)alkyl, —S—(C₂-C₁₅)alkenyl, —S—(C₂-C₁₅)alkynyl, (C₃-C₈)cycloalkyl, —O—(C₃-C₈)cycloalkyl, —S—(C₃-C₈)cycloalkyl, -halo, —NRR, —NO₂, —CN, —C(O)R, —C(S)R, —C(O)OR, —C(S)OR, —SC(S)R, —OC(S)R, —C(O)NRR, —C(S)NRR, —C(O)NR(OR), —C(S)NR(OR), —C(O)NR(SR), —C(S)NR(SR), —CH(CN)₂, —CH[C(O)R]₂, —CH[C(S)R]₂, —CH[C(O)OR]₂, —CH[C(S)OR]₂, —CH[C(O)SR]₂, —CH[C(S)SR]₂, —NRC(O)R, —NRC(O)OR, —S(O)—R, —S(O)OR, and —S(O)NRR;
wherein:
each R is independently selected from —H, (C₁-C₁₀)alkyl, (C₂-C₁₀)alkenyl, (C₂-C₁₀)alkynyl, (C₃-C₈)cycloalkyl, aryl or substituted aryl;
or a pharmaceutically acceptable salt, a prodrug, a tautomer or a solvate thereof;
for use as a therapeutic agent in the attenuation, inhibition, or prevention of conditions associated with IL-6 expression.

18. A method for treating, attenuating, inhibiting, or preventing a condition associated with IL-6 expression in a subject in need thereof, the method comprising administering a therapeutically effective amount of a compound of Formula III: 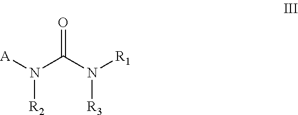
wherein:
A is an arene or a heteroarene;
R₁ is a (C₄₋₁₅)-branched alkyl; (C₃₋₈)cycloalkyl; (C₃₋₈)cycloalkyl having at least one substituent selected from (C₁₋₁₀)alkyl, (C₄₋₁₀)branched alkyl, —O—(C₁-C₁₀)alkyl, —S—(C₁-C₁₀)alkyl, —NRR, —NO₂, —CN, —C(O)R, —C(S)R, —C(O)OR, —C(S)OR, —SC(S)R, —OC(S)R, —C(O)NRR, —C(S)NRR, —C(O)NR(OR), —C(S)NR(OR), —C(O)NR(SR), —C(S)NR(SR), —CH(CN)₂, —CH[C(O)R]₂, —CH[C(S)R]₂, —CH[C(O)OR]₂, —CH[C(S)OR]₂, —CH[C(O)SR]₂, —CH[C(S)SR]₂, —NRC(O)R, —NRC(O)OR, —S(O)—R, —S(O)OR, and —S(O)NRR; (C₆₋₁₈)aryl; or (C₆₋₁₈)aryl having at least one substituent selected from (C₁₋₁₀)alkyl, (C₄₋₁₀)branched alkyl, —O—(C₁-C₁₀)alkyl, —S—(C₁-C₁₀)alkyl, —NRR, —NO₂, —CN, —C(O)R, —C(S)R, —C(O)OR, —C(S)OR, —SC(S)R, —OC(S)R, —C(O)NRR, —C(S)NRR, —C(O)NR(OR), —C(S)NR(OR), —C(O)NR(SR), —C(S)NR(SR), —CH(CN)₂, —CH[C(O)R]₂, —CH[C(S)R]₂, —CH[C(O)OR]₂, —CH[C(S)OR]₂, —CH[C(O)SR]₂, —CH[C(S)SR]₂, —NRC(O)R, —NRC(O)OR, —S(O)—R, —S(O)OR, and —S(O)NRR;
R₂ and R₃ are independently hydrogen, (C₁₋₁₀)alkyl, (C₄₋₁₀)branched alkyl, (C₃₋₈)cycloalkyl; (C₃₋₈)cycloalkyl having at least one substituent selected from (C₁₋₁₀)alkyl, (C₄₋₁₀)branched alkyl, —O—(C₁-C₁₀)alkyl, —S—(C₁-C₁₀)alkyl, —NRR, —NO₂, —CN, —C(O)R, —C(S)R, —C(O)OR, —C(S)OR, —SC(S)R, —OC(S)R, —C(O)NRR, —C(S)NRR, —C(O)NR(OR), —C(S)NR(OR), —C(O)NR(SR), —C(S)NR(SR), —CH(CN)₂, —CH[C(O)R]₂, —CH[C(S)R]₂, —CH[C(O)OR]₂, —CH[C(S)OR]₂, —CH[C(O)SR]₂, —CH[C(S)SR]₂, —NRC(O)R, —NRC(O)OR, —S(O)—R, —S(O)OR, and —S(O)NRR; (C₆₋₁₈)aryl; or (C₆₋₁₈)aryl having at least one substituent selected from (C₁₋₁₀)alkyl, (C₄₋₁₀)branched alkyl, —O—(C₁-C₁₀)alkyl, —S—(C₁-C₁₀)alkyl, —NRR, —NO₂, —CN, —C(O)R, —C(S)R, —C(O)OR, —C(S)OR, —SC(S)R, —OC(S)R, —C(O)NRR, —C(S)NRR, —C(O)NR(OR), —C(S)NR(OR), —C(O)NR(SR), —C(S)NR(SR), —CH(CN)₂, —CH[C(O)R]₂, —CH[C(S)R]₂, —CH[C(O)OR]₂, —CH[C(S)OR]₂, —CH[C(O)SR]₂, —CH[C(S)SR]₂, —NRC(O)R, —NRC(O)OR, —S(O)—R, —S(O)OR, and —S(O)NRR; or
R₂ and R₃ are linked together to form a nitrogen containing ring system, wherein the nitrogen containing ring system is optionally substituted by at least one substituent selected from (C₁₋₁₀)alkyl, (C₄₋₁₀)branched alkyl, —O—(C₁-C₁₀)alkyl, —S—(C₁-C₁₀)alkyl, —NRR, —NO₂, —CN, —C(O)R, —C(S)R, —C(O)OR, —C(S)OR, —SC(S)R, —OC(S)R, —C(O)NRR, —C(S)NRR, —C(O)NR(OR), —C(S)NR(OR), —C(O)NR(SR), —C(S)NR(SR), —CH(CN)₂, —CH[C(O)R]₂, —CH[C(S)R]₂, —CH[C(O)OR]₂, —CH[C(S)OR]₂, —CH[C(O)SR]₂, —CH[C(S)SR]₂, —NRC(O)R, —NRC(O)OR, —S(O)—R, —S(O)OR, and —S(O)NRR;
wherein:
A is optionally substituted with one or more substituents selected from the group of (C₁-C₁₅)alkyl, (C₂-C₁₅)alkenyl, (C₂-C₁₅)alkynyl, —O—(C₁-C₁₅)alkyl, —O—(C₂-C₁₅) alkenyl, —O—(C₂-C₁₅) alkynyl, —S—(C₁-C₁₅)alkyl, —S—(C₂-C₁₅)alkenyl, —S—(C₂-C₁₅)alkynyl, (C₃-C₈)cycloalkyl, —O—(C₃-C₈)cycloalkyl, —S—(C₃-C₈)cycloalkyl, -halo, —NRR, —NO₂, —CN, —C(O)R, —C(S)R, —C(O)OR, —C(S)OR, —SC(S)R, —OC(S)R, —C(O)NRR, —C(S)NRR, —C(O)NR(OR), —C(S)NR(OR), —C(O)NR(SR), —C(S)NR(SR), —CH(CN)₂, —CH[C(O)R]₂, —CH[C(S)R]₂, —CH[C(O)OR]₂, —CH[C(S)OR]₂, —CH[C(O)SR]₂, —CH[C(S)SR]₂, —NRC(O)R, —NRC(O)OR, —S(O)—R, —S(O)OR, and —S(O)NRR;
wherein:
each R is independently selected from —H, (C₁-C₁₀)alkyl, (C₂-C₁₀)alkenyl, (C₂-C₁₀)alkynyl, (C₃-C₈)cycloalkyl, aryl or substituted aryl;
or a pharmaceutically acceptable salt, a prodrug, a tautomer or a solvate thereof.

19. A pharmaceutical composition comprising a compound as defined in any one of claims 15 to 17, and a pharmaceutically acceptable carrier.

20. A medicament for use in treating, attenuating, inhibiting and/or preventing inflammation and inflammation-related pathologies, the medicament comprising a compound as defined in any one of claims 15 to 17, and a pharmaceutically acceptable carrier.

21. A medicament for use in treating a condition associated with IL-6 expression, the medicament comprising a compound as defined in any one of claims 15 to 17, and a pharmaceutically acceptable carrier.

22. A compound of Formula III: 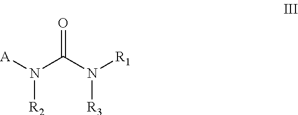
wherein:
A is an arene;
R₁ is a (C₄₋₁₅)-branched alkyl; (C₃₋₈)cycloalkyl; or (C₃₋₈)cycloalkyl having at least one substituent selected from (C₁₋₁₀)alkyl, (C₄₋₁₀)branched alkyl, —O—(C₁-C₁₀)alkyl, —S—(C₁-C₁₀)alkyl, —NRR, —CN, —C(O)R, —C(O)OR, —C(O)NRR, —NRC(O)R, —S(O)—R, —S(O)OR, and —S(O)NRR;
R₂ and R₃ are independently hydrogen, (C₁₋₁₀)alkyl, (C₄₋₁₀)branched alkyl, (C₃₋₈)cycloalkyl; (C₃₋₈)cycloalkyl having at least one substituent selected from (C₁₋₁₀)alkyl, (C₄₋₁₀)branched alkyl, —O—(C₁-C₁₀)alkyl, —S—(C₁-C₁₀)alkyl, —NRR, —CN, —C(O)R, —C(O)OR, —C(O)NRR, —S(O)—R, —S(O)OR, and —S(O)NRR; or
R₂ and R₃ are linked together to form a nitrogen containing ring system, wherein the nitrogen containing ring system is optionally substituted by at least one substituent selected from (C₁₋₁₀)alkyl, (C₄₋₁₀)branched alkyl, —O—(C₁-C₁₀)alkyl, —S—(C₁-C₁₀)alkyl, —NRR, CN, —C(O)R, —C(O)OR, —C(O)NRR, —S(O)—R, —S(O)OR, and —S(O)NRR;
wherein:
A is optionally substituted with one or more substituents selected from the group of (C₁-C₁₅)alkyl, —O—(C₁-C₁₅)alkyl, —S—(C₁-C₁₅)alkyl, (C₃-C₈)cycloalkyl, —O—(C₃-C₈)cycloalkyl, —S—(C₃-C₈)cycloalkyl, -halo, —NRR, —CN, —C(O)R, —C(O)OR, —C(O)NRR, —NRC(O)R, —NRC(O)OR, —S(O)—R, —S(O)OR, and —S(O)NRR;
wherein:
each R is independently selected from —H, (C₁-C₁₀)alkyl,
or a pharmaceutically acceptable salt, a prodrug, a tautomer or a solvate thereof.

23. A compound of Formula III: 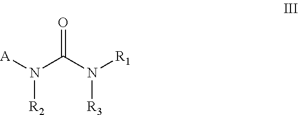
wherein:
A is an arene;
R₁ is a (C₄₋₁₅)-branched alkyl; (C₃₋₈)cycloalkyl; or (C₃₋₈)cycloalkyl having at least one substituent selected from (C₁₋₁₀)alkyl, (C₄₋₁₀)branched alkyl, —O—(C₁-C₀)alkyl, —S—(C₁-C₁₀)alkyl, —NRR, —CN, —C(O)R, —C(O)OR, —C(O)NRR, —NRC(O)R, —S(O)—R, —S(O)OR, and —S(O)NRR;
R₂ and R₃ are independently hydrogen, (C₁₋₁₀)alkyl, (C₄₋₁₀)branched alkyl, (C₃₋₈)cycloalkyl; (C₃₋₈)cycloalkyl having at least one substituent selected from (C₁₋₁₀)alkyl, (C₄₋₁₀)branched alkyl, —O—(C₁-C₁₀)alkyl, —S—(C₁-C₁₀)alkyl, —NRR, —CN, —C(O)R, —C(O)OR, —C(O)NRR, —S(O)—R, —S(O)OR, and —S(O)NRR; or
R₂ and R₃ are linked together to form a nitrogen containing ring system, wherein the nitrogen containing ring system is optionally substituted by at least one substituent selected from (C₁₋₁₀)alkyl, (C₄₋₁₀)branched alkyl, —O—(C₁-C₁₀)alkyl, —S—(C₁-C₁₀)alkyl, —NRR, CN, —C(O)R, —C(O)OR, —C(O)NRR, —S(O)—R, —S(O)OR, and —S(O)NRR;
wherein:
A is optionally substituted with one or more substituents selected from the group of (C₁-C₁₅)alkyl, —O—(C₁-C₁₅)alkyl, —S—(C₁-C₁₅)alkyl, (C₃-C₈)cycloalkyl, —O—(C₃-C₈)cycloalkyl, —S—(C₃-C₈)cycloalkyl, -halo, —NRR, —CN, —C(O)R, —C(O)OR, —C(O)NRR, —NRC(O)R, —NRC(O)OR, —S(O)—R, —S(O)OR, and —S(O)NRR;
wherein:
each R is independently selected from —H, (C₁-C₁₀)alkyl,
or a pharmaceutically acceptable salt, a prodrug, a tautomer or a solvate thereof;
for use as an anti-proliferative agent in the attenuation, inhibition, or prevention of conditions associated with IL-6 expression.

24. A compound of Formula III: 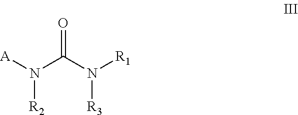
wherein:
A is an arene;
R₁ is a (C₄₋₁₅)-branched alkyl; (C₃₋₈)cycloalkyl; or (C₃₋₈)cycloalkyl having at least one substituent selected from (C₁₋₁₀)alkyl, (C₄₋₁₀)branched alkyl, —O—(C₁-C₁₀)alkyl, —S—(C₁-C₁₀)alkyl, —NRR, —CN, —C(O)R, —C(O)OR, —C(O)NRR, —NRC(O)R, —S(O)—R, —S(O)OR, and —S(O)NRR;
R₂ and R₃ are independently hydrogen, (C₁₋₁₀)alkyl, (C₄₋₁₀)branched alkyl, (C₃₋₈)cycloalkyl; (C₃₋₈)cycloalkyl having at least one substituent selected from (C₁₋₁₀)alkyl, (C₄₋₁₀)branched alkyl, —O—(C₁-C₁₀)alkyl, —S—(C₁-C₁₀)alkyl, —NRR, —CN, —C(O)R, —C(O)OR, —C(O)NRR, —S(O)—R, —S(O)OR, and —S(O)NRR; or
R₂ and R₃ are linked together to form a nitrogen containing ring system, wherein the nitrogen containing ring system is optionally substituted by at least one substituent selected from (C₁₋₁₀)alkyl, (C₄₋₁₀)branched alkyl, —O—(C₁-C₁₀)alkyl, —S—(C₁-C₁₀)alkyl, —NRR, CN, —C(O)R, —C(O)OR, —C(O)NRR, —S(O)—R, —S(O)OR, and —S(O)NRR;
wherein:
A is optionally substituted with one or more substituents selected from the group of (C₁-C₁₅)alkyl, —O—(C₁-C₁₅)alkyl, —S—(C₁-C₁₅)alkyl, (C₃-C₈)cycloalkyl, —O—(C₃-C₈)cycloalkyl, —S—(C₃-C₈)cycloalkyl, -halo, —NRR, —CN, —C(O)R, —C(O)OR, —C(O)NRR, —NRC(O)R, —NRC(O)OR, —S(O)—R, —S(O)OR, and —S(O)NRR;
wherein:
each R is independently selected from —H, (C₁-C₁₀)alkyl,
or a pharmaceutically acceptable salt, a prodrug, a tautomer or a solvate thereof;
for use as a therapeutic agent in the attenuation, inhibition, or prevention of conditions associated with IL-6 expression.

25. A method for treating, attenuating, inhibiting, or preventing a condition associated with IL-6 expression in a subject in need thereof, the method comprising administering a therapeutically effective amount of a compound of Formula III: 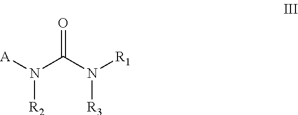
wherein:
A is an arene;
R₁ is a (C₄₋₁₅)-branched alkyl; (C₃₋₈)cycloalkyl; or (C₃₋₈)cycloalkyl having at least one substituent selected from (C₁₋₁₀)alkyl, (C₄₋₁₀)branched alkyl, —O—(C₁-C₁₀)alkyl, —S—(C₁-C₁₀)alkyl, —NRR, —CN, —C(O)R, —C(O)OR, —C(O)NRR, —NRC(O)R, —S(O)—R, —S(O)OR, and —S(O)NRR;
R₂ and R₃ are independently hydrogen, (C₁₋₁₀)alkyl, (C₄₋₁₀)branched alkyl, (C₃₋₈)cycloalkyl; (C₃₋₈)cycloalkyl having at least one substituent selected from (C₁₋₁₀)alkyl, (C₄₋₁₀)branched alkyl, —O—(C₁-C₁₀)alkyl, —S—(C₁-C₁₀)alkyl, —NRR, —CN, —C(O)R, —C(O)OR, —C(O)NRR, —S(O)—R, —S(O)OR, and —S(O)NRR; or
R₂ and R₃ are linked together to form a nitrogen containing ring system, wherein the nitrogen containing ring system is optionally substituted by at least one substituent selected from (C₁₋₁₀)alkyl, (C₄₋₁₀)branched alkyl, —O—(C₁-C₁₀)alkyl, —S—(C₁-C₁₀)alkyl, —NRR, CN, —C(O)R, —C(O)OR, —C(O)NRR, —S(O)—R, —S(O)OR, and —S(O)NRR;
wherein:
A is optionally substituted with one or more substituents selected from the group of (C₁-C₁₅)alkyl, —O—(C₁-C₁₅)alkyl, —S—(C₁-C₁₅)alkyl, (C₃-C₈)cycloalkyl, —O—(C₃-C₈)cycloalkyl, —S—(C₃-C₈)cycloalkyl, -halo, —NRR, —CN, —C(O)R, —C(O)OR, —C(O)NRR, —NRC(O)R, —NRC(O)OR, —S(O)—R, —S(O)OR, and —S(O)NRR;
wherein:
each R is independently selected from —H, (C₁-C₁₀)alkyl,
or a pharmaceutically acceptable salt, a prodrug, a tautomer or a solvate thereof.

26. A pharmaceutical composition comprising a compound as defined in any one of claims 22 to 24, and a pharmaceutically acceptable carrier.

27. A medicament for use in treating, attenuating, inhibiting and/or preventing inflammation and inflammation-related pathologies, the medicament comprising a compound as defined in any one of claims 22 to 24, and a pharmaceutically acceptable carrier.

28. A medicament for use in treating a condition associated with IL-6 expression, the medicament comprising a compound as defined in any one of claims 22 to 24, and a pharmaceutically acceptable carrier.

29. A compound of Formula IV: 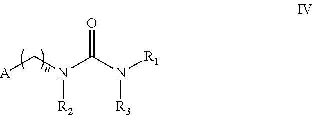
wherein:
A is an arene;
R₁ is a (C₄₋₁₅)alkyl; (C₄₋₁₅)-branched alkyl; (C₃₋₈)cycloalkyl; alk(C₃₋₈)cycloalkyl, (C₃₋₈)cycloalkenyl, alk(C₃₋₈)cycloalkenyl, (C₃₋₈)cycloalkyl having at least one substituent selected from (C₁₋₁₀)alkyl, (C₄₋₁₀)branched alkyl, —O—(C₁-C₁₀)alkyl, ROR—; —S—(C₁-C₁₀)alkyl, —NRR, —CN, —C(O)R, —C(O)OR, —C(O)NRR, —NRC(O)R, —S(O)—R, —S(O)OR, and —S(O)NRR; or (C₃₋₈)cycloalkenyl having at least one substituent selected from (C₁₋₁₀)alkyl, (C₄₋₁₀)branched alkyl, —O—(C₁-C₁₀)alkyl, ROR—; —S—(C₁-C₁₀)alkyl, —NRR, —CN, —C(O)R, —C(O)OR, —C(O)NRR, —NRC(O)R, —S(O)—R, —S(O)OR, and —S(O)NRR;
R₂ and R₃ are independently hydrogen, (C₁₋₁₀)alkyl, (C₄₋₁₀)branched alkyl, (C₃₋₈)cycloalkyl; (C₃₋₈)cycloalkyl having at least one substituent selected from (C₁₋₁₀)alkyl, (C₄₋₁₀)branched alkyl, —O—(C₁-C₁₀)alkyl, —S—(C₁-C₁₀)alkyl, —NRR, —CN, —C(O)R, —C(O)OR, —C(O)NRR, —S(O)—R, —S(O)OR, and —S(O)NRR; or
R₂ and R₃ are linked together to form a nitrogen containing ring system, wherein the nitrogen containing ring system is optionally substituted by at least one substituent selected from (C₁₋₁₀)alkyl, (C₄₋₁₀)branched alkyl, —O—(C₁-C₁₀)alkyl, —S—(C₁-C₁₀)alkyl, —NRR, CN, —C(O)R, —C(O)OR, —C(O)NRR, —S(O)—R, —S(O)OR, and —S(O)NRR;
n is 0, 1 or 2;
wherein:
A is optionally substituted with one or more substituents selected from the group of (C₁-C₁₅)alkyl, —O—(C₁-C₁₅)alkyl, —S—(C₁-C₁₅)alkyl, (C₃-C₈)cycloalkyl, —O—(C₃-C₈)cycloalkyl, —S—(C₃-C₈)cycloalkyl, -halo, —NRR, —CN, —C(O)R, —C(O)OR, —C(O)NRR, —NRC(O)R, —NRC(O)OR, —S(O)—R, —S(O)OR, and —S(O)NRR;
wherein:
each R is independently selected from —H, (C₁-C₁₀)alkyl,
or a pharmaceutically acceptable salt, a prodrug, a tautomer or a solvate thereof.

30. The compound of claim 29, including:

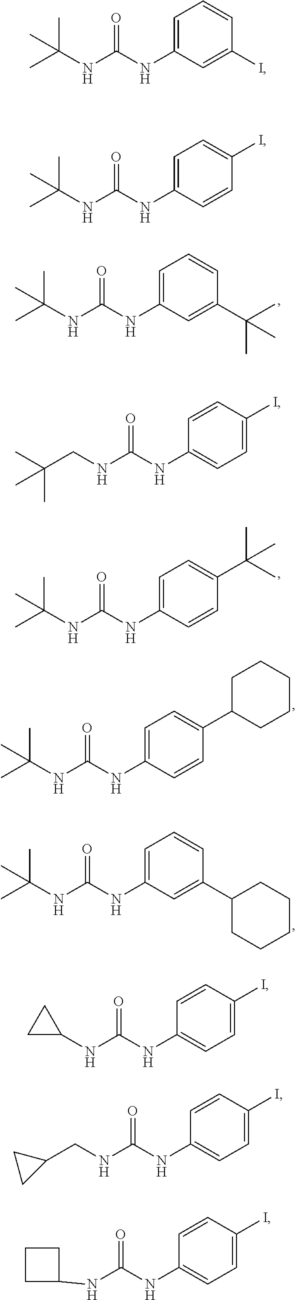

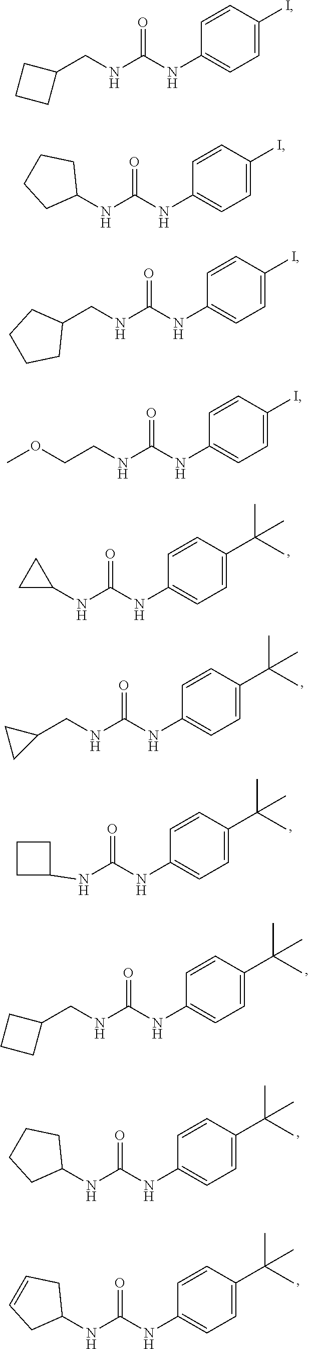

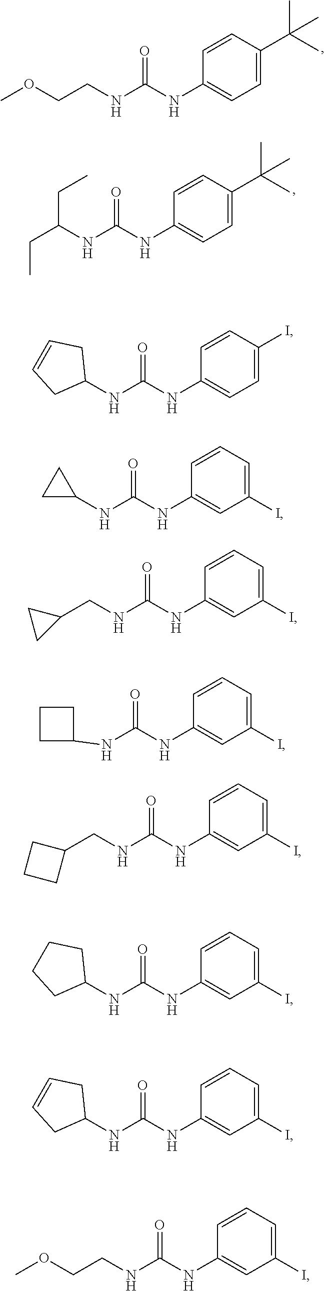

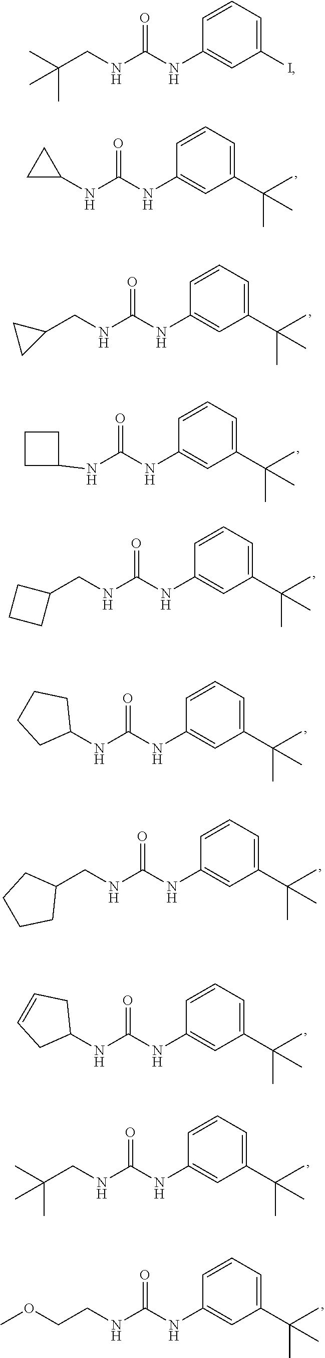

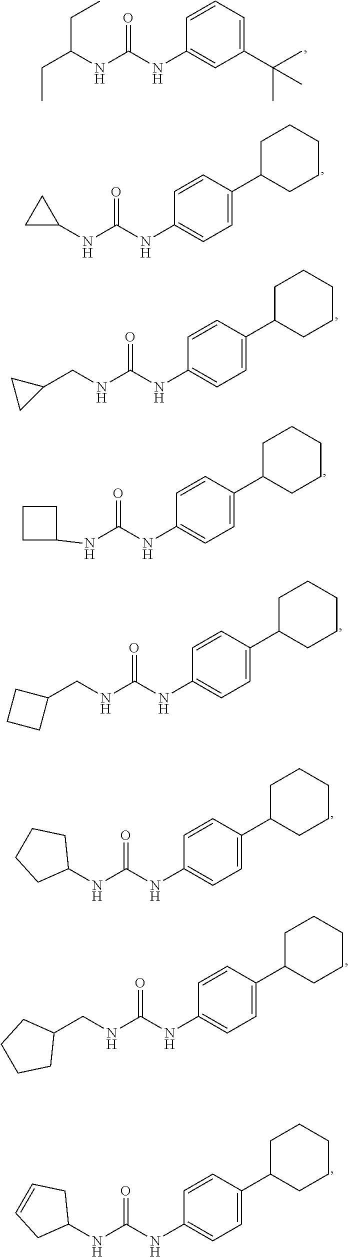

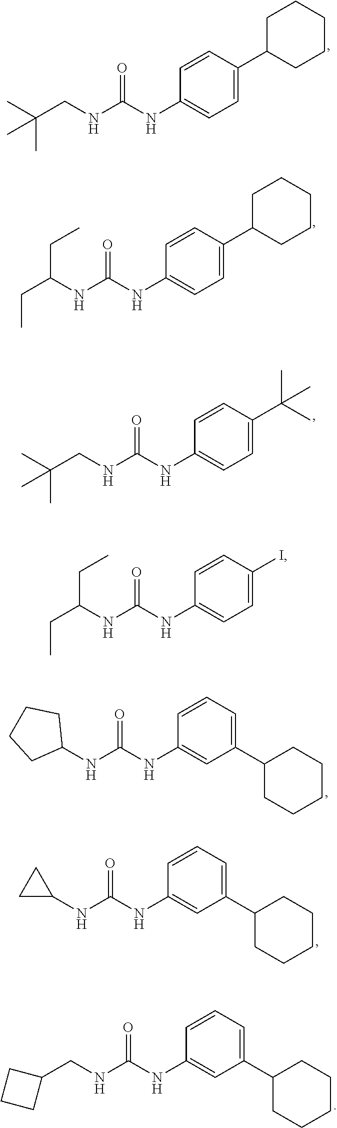

31. A pharmaceutical composition comprising a compound as defined in claim 29 or 30, and a pharmaceutically acceptable carrier.

32. A medicament for use in treating, attenuating, inhibiting and/or preventing inflammation and inflammation-related pathologies, the medicament comprising a compound as defined in claim 29 or 30, and a pharmaceutically acceptable carrier.

33. A medicament for use in treating a condition associated with IL-6 expression, the medicament comprising a compound as defined in claim 29 or 30, and a pharmaceutically acceptable carrier.

34. A compound of Formula V: 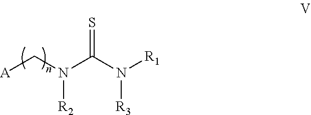
wherein:
A is an arene;
R₁ is a (C₁-5)alkyl; (C₄₋₁₅)-branched alkyl; (C₃₋₈)cycloalkyl; alk(C₃₋₈)cycloalkyl, (C₃₋₈)cycloalkenyl, alk(C₃₋₈)cycloalkenyl, (C₃₋₈)cycloalkyl having at least one substituent selected from (C₁₋₁₀)alkyl, (C₄₋₁₀)branched alkyl, —O—(C₁-C₁₀)alkyl, ROR—; —S—(C₁-C₁₀)alkyl, —NRR, —CN, —C(O)R, —C(O)OR, —C(O)NRR, —NRC(O)R, —S(O)—R, —S(O)OR, and —S(O)NRR; or (C₃₋₈)cycloalkenyl having at least one substituent selected from (C₁₋₁₀)alkyl, (C₄₋₁₀)branched alkyl, —O—(C₁-C₁₀)alkyl, ROR—; —S—(C₁-C₁₀)alkyl, —NRR, —CN, —C(O)R, —C(O)OR, —C(O)NRR, —NRC(O)R, —S(O)—R, —S(O)OR, and —S(O)NRR;
R₂ and R₃ are independently hydrogen, (C₁₋₁₀)alkyl, (C₄₋₁₀)branched alkyl, (C₃₋₈)cycloalkyl; (C₃₋₈)cycloalkyl having at least one substituent selected from (C₁₋₁₀)alkyl, (C₄₋₁₀)branched alkyl, —O—(C₁-C₁₀)alkyl, —S—(C₁-C₁₀)alkyl, —NRR, —CN, —C(O)R, —C(O)OR, —C(O)NRR, —S(O)—R, —S(O)OR, and —S(O)NRR; or
R₂ and R₃ are linked together to form a nitrogen containing ring system, wherein the nitrogen containing ring system is optionally substituted by at least one substituent selected from (C₁₋₁₀)alkyl, (C₄₋₁₀)branched alkyl, —O—(C₁-C₁₀)alkyl, —S—(C₁-C₁₀)alkyl, —NRR, CN, —C(O)R, —C(O)OR, —C(O)NRR, —S(O)—R, —S(O)OR, and —S(O)NRR;
n is 0, 1 or 2;
wherein:
A is optionally substituted with one or more substituents selected from the group of (C₁-C₁₅)alkyl, —O—(C₁-C₁₅)alkyl, —S—(C₁-C₁₅)alkyl, (C₃-C₈)cycloalkyl, —O—(C₃-C₈)cycloalkyl, —S—(C₃-C₈)cycloalkyl, -halo, —NRR, —CN, —C(O)R, —C(O)OR, —C(O)NRR, —NRC(O)R, —NRC(O)OR, —S(O)—R, —S(O)OR, and —S(O)NRR;
wherein:
each R is independently selected from —H, (C₁-C₁₀)alkyl,
or a pharmaceutically acceptable salt, a prodrug, a tautomer or a solvate thereof.

35. The compound of claim 34, including:

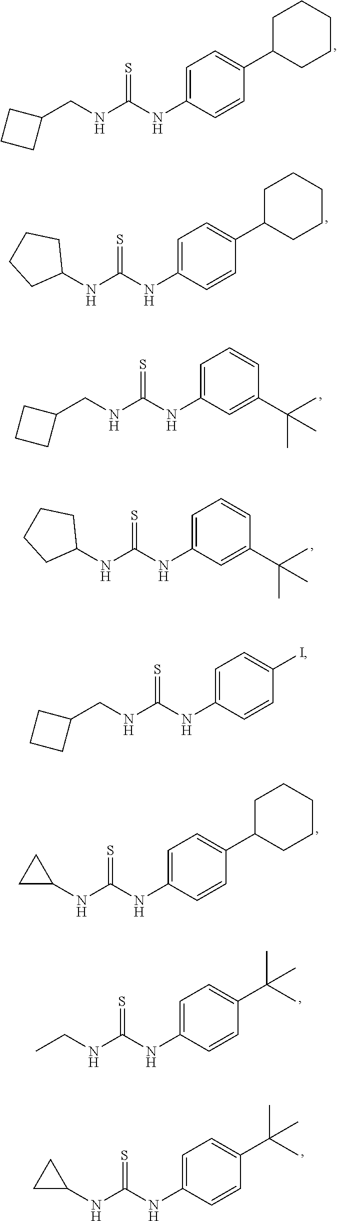

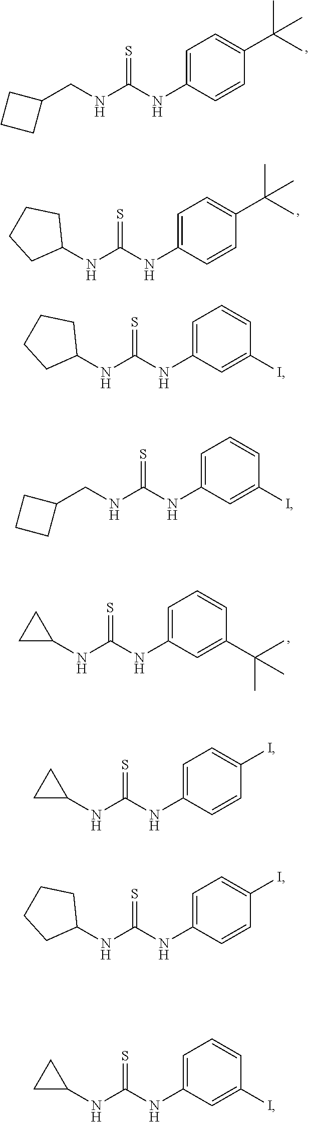

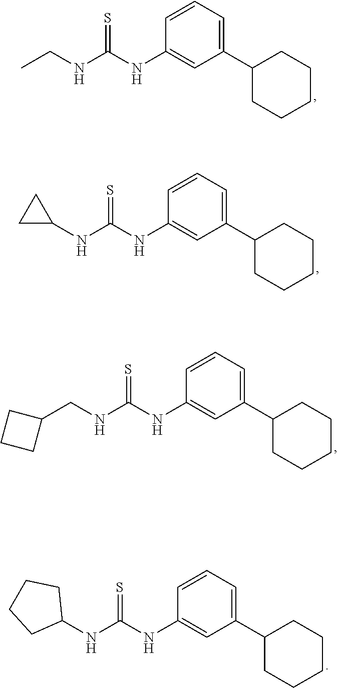 [SMILES: CCNC(=S)Nc1cccc(C2CCCCC2)c1 ; S=C(Nc1cccc(C2CCCCC2)c1)NC1CC1 ; S=C(NCC1CCC1)Nc1cccc(C2CCCCC2)c1 ; S=C(Nc1cccc(C2CCCCC2)c1)NC1CCCC1]

36. A pharmaceutical composition comprising a compound as defined in claim 34 or 35, and a pharmaceutically acceptable carrier.

37. A medicament for use in treating, attenuating, inhibiting and/or preventing inflammation and inflammation-related pathologies, the medicament comprising a compound as defined in claim 34 or 35, and a pharmaceutically acceptable carrier.

38. A medicament for use in treating a condition associated with IL-6 expression, the medicament comprising a compound as defined in claim 34 or 35, and a pharmaceutically acceptable carrier.

39. A compound of Formula VI: 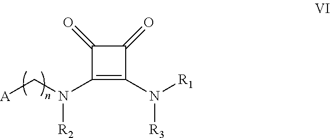
wherein:
A is an arene;
R₁ is a (C₁₋₁₅)alkyl; (C₄₋₁₅)-branched alkyl; (C₃₋₈)cycloalkyl; alk(C₃₋₈)cycloalkyl, (C₃₋₈)cycloalkenyl, alk(C₃₋₈)cycloalkenyl, (C₃₋₈)cycloalkyl having at least one substituent selected from (C₁₋₁₀)alkyl, (C₄₋₁₀)branched alkyl, —O—(C₁-C₁₀)alkyl, ROR—; —S—(C₁-C₁₀)alkyl, —NRR, —CN, —C(O)R, —C(O)OR, —C(O)NRR, —NRC(O)R, —S(O)—R, —S(O)OR, and —S(O)NRR; or (C₃₋₈)cycloalkenyl having at least one substituent selected from (C₁₋₁₀)alkyl, (C₄₋₁₀)branched alkyl, —O—(C₁-C₁₀)alkyl, ROR—; —S—(C₁-C₁₀)alkyl, —NRR, —CN, —C(O)R, —C(O)OR, —C(O)NRR, —NRC(O)R, —S(O)—R, —S(O)OR, and —S(O)NRR;
R₂ and R₃ are independently hydrogen, (C₁₋₁₀)alkyl, (C₄₋₁₀)branched alkyl, (C₃₋₈)cycloalkyl; (C₃₋₈)cycloalkyl having at least one substituent selected from (C₁₋₁₀)alkyl, (C₄₋₁₀)branched alkyl, —O—(C₁-C₁₀)alkyl, —S—(C₁-C₁₀)alkyl, —NRR, —CN, —C(O)R, —C(O)OR, —C(O)NRR, —S(O)—R, —S(O)OR, and —S(O)NRR; or
R₂ and R₃ are linked together to form a nitrogen containing ring system, wherein the nitrogen containing ring system is optionally substituted by at least one substituent selected from (C₁₋₁₀)alkyl, (C₄₋₁₀)branched alkyl, —O—(C₁-C₁₀)alkyl, —S—(C₁-C₁₀)alkyl, —NRR, CN, —C(O)R, —C(O)OR, —C(O)NRR, —S(O)—R, —S(O)OR, and —S(O)NRR;
n is 0, 1 or 2;
wherein:
A is optionally substituted with one or more substituents selected from the group of (C₁-C₁₅)alkyl, —O—(C₁-C₁₅)alkyl, —S—(C₁-C₁₅)alkyl, (C₃-C₈)cycloalkyl, —O—(C₃-C₈)cycloalkyl, —S—(C₃-C₈)cycloalkyl, -halo, —NRR, —CN, —C(O)R, —C(O)OR, —C(O)NRR, —NRC(O)R, —NRC(O)OR, —S(O)—R, —S(O)OR, and —S(O)NRR;
wherein:
each R is independently selected from —H, (C₁-C₁₀)alkyl,
or a pharmaceutically acceptable salt, a prodrug, a tautomer or a solvate thereof.

40. The compound of claim 39, including:

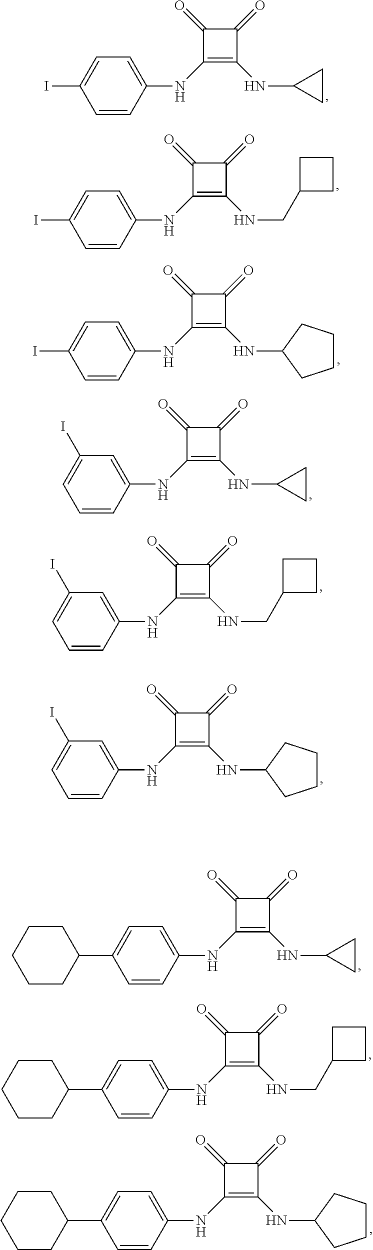

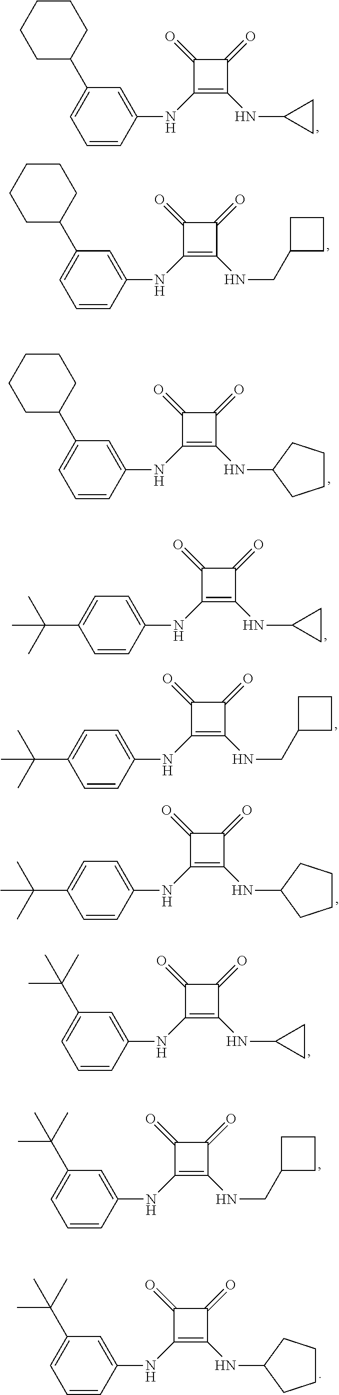

41. A pharmaceutical composition comprising a compound as defined in claim 39 or 40, and a pharmaceutically acceptable carrier.

42. A medicament for use in treating, attenuating, inhibiting and/or preventing inflammation and inflammation-related pathologies, the medicament comprising a compound as defined in claim 39 or 40, and a pharmaceutically acceptable carrier.

43. A medicament for use in treating a condition associated with IL-6 expression, the medicament comprising a compound as defined in claim 39 or 40, and a pharmaceutically acceptable carrier.
